(19) United States
(12) Patent Application Publication
(10) Pub. No.: US 2023/0151366 A1
(43) Pub. Date: May 18, 2023

(54) COMPOSITIONS AND METHODS OF INHIBITING SEVERE ACUTE RESPIRATORY SYNDROME CORONAVIRUS 2 (SARS-CoV-2)

(71) Applicant: Ionis Pharmaceuticals, Inc., Carlsbad, CA (US)

(72) Inventors: Susan M. Freier, San Diego, CA (US); Eric E. Swayze, Encinitas, CA (US); Robert J. Prill, San Diego, CA (US)

(73) Assignee: Ionis Pharmaceuticals, Inc., Carlsbad, CA (US)

(21) Appl. No.: 17/919,346

(22) PCT Filed: Apr. 16, 2021
(86) PCT No.: PCT/US2021/027615

§ 371 Date: Oct. 17, 2022

Related U.S. Application Data
(60) Provisional application No. 63/011,942, filed on Apr. 17, 2020.

(51) Int. Cl. C12N 15/113; A61P 31/14

(52) U.S. Cl. CPC C12N 15/1131; A61P 31/14; C12N 2310/3341; C12N 2310/322; C12N 2310/315; C12N 2310/11; C12N 2310/341

(57) ABSTRACT

The present embodiments provide methods, compounds, and compositions useful for inhibiting or reducing SARS-CoV-2 replication, infectivity, viral titer, or viral load, which can be useful for preventing or treating COVID-19 in an individual.

### SEQUENCE LISTING

The present application is being filed along with a Sequence Listing in electronic format. The Sequence Listing is provided as a file entitled BIOL0391WOSEQ_ST25.txt created Apr. 12, 2021, which is 194 kb in size. The information in the electronic format of the sequence listing is incorporated herein by reference in its entirety.

### FIELD

Embodiments described herein relate to compounds, compositions, and methods for inhibiting the Severe Acute Respiratory Syndrome Coronavirus (SARS-CoV-2) and preventing or treating its associated disease, Coronavirus Disease 2019 (COVID-19).

### BACKGROUND

The Severe Acute Respiratory Syndrome Coronavirus (SARS-CoV-2) is a global pandemic that has infected over 2 million people and killed over 139,000 people worldwide as of Apr. 16, 2020 according to the Johns Hopkins Coronavirus Resource Center Dashboard. These numbers represent the total confirmed cases, but the true numbers of infections and deaths are surely higher due to underreporting and the shortage of testing. The true number of infections has been estimated up to 10 times higher.

SARS-CoV-2 is highly contagious. It has a long incubation period of 1 to 14 days of contagiousness before an infected individual shows symptoms, if at all. A recent report indicated that COVID-19 may be most contagious 1 to 2 days before symptoms appear. Infected but asymptomatic individuals are dubbed superspreaders. The infection is spreading exponentially and the doubling time of the number of infected persons was estimated at approximately 2 days in the United States in March 2020 but has recently been estimated at 6.5 days on Apr. 7, 2020.

The most common symptoms of COVID-19 are fever, fatigue, and dry cough. For some individuals, especially the elderly and people with underlying medical conditions, COVID-19 can cause difficulty breathing leading to hospitalization and intubation with a ventilator because patients can no longer breathe on their own. COVID-19 is fatal when patients succumb to lung damage, respiratory failure, and/or pneumonia.

In addition to the public health crisis caused by SARS-CoV-2 and COVID-19, the pandemic has also caused severe economic duress. In the United States, where states have mandated sheltering at home and social distancing to flatten the curve of infection, 22 million people have become unemployed ever since a national energy over COVID-19 was declared on Mar. 13, 2020.

Currently, no vaccine or approved therapy for SARS-CoV-2 and COVID-19 exists. Although social distancing and sheltering at home appears to be dampening the forecast on the number of COVID-19 deaths in the United States from the 100,000-240,000 range to approximately 60,000 according to a model from the University of Washington, there is an urgent need for a therapeutic against SARS-CoV-2 to stop the pandemic.

### SUMMARY

Embodiments described herein relate to the design and synthesis of compounds and compositions that can be administered to inhibit the replication or infectivity of SARS-CoV-2 and to prevent or treat (COVID-19).

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ION number indicate a combination of nucleobase sequence, chemical modification, and motif.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE, MOE, methoxyethyl, and 2′-O(CH₂)₂—OCH₃) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within ±10% of a value. For example, if it is stated, “the compounds affected about 70% inhibition of SARS-CoV-2”, it is implied that SARS-CoV-2 levels are inhibited within a range of 60% and 80%.

“Administration” or “administering” refers to routes of introducing a compound or composition provided herein to an individual to perform its intended function. An example of a route of administration that can be used includes, but is not limited to inhalation such as through a nebulizer or inhaler.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′.

“cEt nucleoside” means a nucleoside comprising a cEt modified sugar moiety.

“Chemical modification” in a compound describes the substitutions or changes through chemical reaction, of any of the units in the compound relative to the original state of such unit. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Coronavirus Disease 2019 (COVID-19)” refers to the disease caused by the Severe Acute Respiratory Syndrome Coronavirus (SARS-CoV-2) and includes, but is not limited to, one or more symptoms associated with SARS-CoV-2 infection such as respiratory illness, difficulty breathing, fever, cough, fatigue, aches and pains, sore throat, runny nose, diarrhea, loss of taste or smell, and nasal congestion.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” means chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded antisense compound” means an antisense compound comprising two oligomeric compounds that are complementary to each other and form a duplex, and wherein one of the two said oligomeric compounds comprises an oligonucleotide.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to, the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Linker-nucleoside” means a nucleoside that links an oligonucleotide to a conjugate moiety. Linker-nucleosides are located within the conjugate linker of a compound. Linker-nucleosides are not considered part of the oligonucleotide portion of a compound even if they are contiguous with the oligonucleotide.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely. In the context of preventing COVID-19, “prevent” refers to forestalling the onset, development or progression of any symptoms associated with SARS-CoV-2 infection and/or delaying or forestalling the onset or increase in SARS-CoV-2 replication, infectivity, viral titer, or viral load in the individual.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Severe Acute Respiratory Syndrome Coronavirus (SARS-CoV-2)” refers to all strains and isolates of coronavirus that causes COVID-19.

“SARS-CoV-2 specific inhibitor” refers to any agent capable of specifically inhibiting SARS-CoV-2 RNA and/or SARS-CoV-2 protein expression or activity at the molecular level. For example, SARS-CoV-2 specific inhibitors include nucleic acids (including antisense compounds), peptides, antibodies, small molecules, and other agents capable of inhibiting the expression of SARS-CoV-2 RNA and/or SARS-CoV-2 protein.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” with reference to a target nucleic acid means to reduce or block expression of the target nucleic acid while exhibiting fewer, minimal, or no effects on non-target nucleic acids. Reduction does not necessarily indicate a total elimination of the target nucleic acid's expression.

“Stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary compounds or nucleic acids.

“Targeting” means the specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an individual in order to effect an alteration or improvement of a disease, disorder, or condition in the individual. In the context of treating COVID-19, “treat” refers to improving any symptoms associated with SARS-CoV-2 infection and/or inhibiting or reducing SARS-CoV-2 replication, infectivity, viral titer, or viral load in the individual.

### Certain Embodiments

Certain embodiments provide methods, compounds and compositions for inhibiting or reducing SARS-CoV-2 replication, infectivity, viral titer, or viral load, thereby inhibiting or reducing SARS-CoV-2 replication, infectivity, viral titer, or viral load in the lung cells.

Certain embodiments provide compounds targeted to SARS-CoV-2 RNA. In certain embodiments, the SARS-CoV-2 RNA has the sequence set forth in GENBANK Accession No. NC_045512.2, which is incorporated by reference in its entirety and designated herein as SEQ ID NO: 1. In certain embodiments, the SARS-CoV-2 RNA has the sequence set forth in the complement of GENBANK Accession No. NC_045512.2, designated herein as SEQ ID NO: 2, which is the complement of genomic sequence of severe acute respiratory syndrome coronavirus 2 isolate Wuhan-Hu-1, designated herein as SEQ ID NO: 2 (the complement of GENBANK Accession No. NC_045512.2).

In certain embodiments, the compound is an antisense compound or oligomeric compound. In certain embodiments, the compound is single-stranded. In certain embodiments, the compound is double-stranded.

Certain embodiments provide a compound comprising a modified oligonucleotide consisting of 8 to 80 linked nucleosides and having a nucleobase sequence comprising at least 8 contiguous nucleobases of any of the nucleobase sequences of SEQ ID NOs: 3-599. In certain embodiments, the compound is an antisense compound or oligomeric compound. In certain embodiments, the compound is single-stranded. In certain embodiments, the compound is double-stranded. In certain embodiments, the modified oligonucleotide consists of 10 to 30 linked nucleosides.

Certain embodiments provide a compound comprising a modified oligonucleotide consisting of 9 to 80 linked nucleosides and having a nucleobase sequence comprising at least 9 contiguous nucleobases of any of the nucleobase sequences of SEQ ID NOs: 3-599. In certain embodiments, the compound is an antisense compound or oligomeric compound. In certain embodiments, the compound is single-stranded. In certain embodiments, the compound is double-stranded. In certain embodiments, the modified oligonucleotide consists of 10 to 30 linked nucleosides.

Certain embodiments provide a compound comprising a modified oligonucleotide consisting of 10 to 80 linked nucleosides and having a nucleobase sequence comprising at least 10 contiguous nucleobases of any of the nucleobase sequences of SEQ ID NOs: 3-599. In certain embodiments, the compound is an antisense compound or oligomeric compound. In certain embodiments, the compound is single-stranded. In certain embodiments, the compound is double-stranded. In certain embodiments, the modified oligonucleotide consists of 10 to 30 linked nucleosides.

Certain embodiments provide a compound comprising a modified oligonucleotide consisting of 11 to 80 linked nucleosides and having a nucleobase sequence comprising at least 11 contiguous nucleobases of any of the nucleobase sequences of SEQ ID NOs: 3-599. In certain embodiments, the compound is an antisense compound or oligomeric compound. In certain embodiments, the compound is single-stranded. In certain embodiments, the compound is double-stranded. In certain embodiments, the modified oligonucleotide consists of 11 to 30 linked nucleosides.

Certain embodiments provide a compound comprising a modified oligonucleotide consisting of 12 to 80 linked nucleosides and having a nucleobase sequence comprising at least 12 contiguous nucleobases of any of the nucleobase sequences of SEQ ID NOs: 3-599. In certain embodiments, the compound is an antisense compound or oligomeric compound. In certain embodiments, the compound is single-stranded. In certain embodiments, the compound is double-stranded. In certain embodiments, the modified oligonucleotide consists of 12 to 30 linked nucleosides.

Certain embodiments provide a compound comprising a modified oligonucleotide consisting of 16 to 80 linked nucleosides and having a nucleobase sequence comprising the nucleobase sequence of any one of SEQ ID NOs: 3-470 or 549-588. In certain embodiments, the compound is an antisense compound or oligomeric compound. In certain embodiments, the compound is single-stranded. In certain embodiments, the compound is double-stranded. In certain embodiments, the modified oligonucleotide consists of 16 to 30 linked nucleosides.

Certain embodiments provide a compound comprising a modified oligonucleotide consisting of 18 to 80 linked nucleosides and having a nucleobase sequence comprising the nucleobase sequence of any one of SEQ ID NOs: 471-510. In certain embodiments, the compound is an antisense compound or oligomeric compound. In certain embodiments, the compound is single-stranded. In certain embodiments, the compound is double-stranded. In certain embodiments, the modified oligonucleotide consists of 18 to 30 linked nucleosides.

Certain embodiments provide a compound comprising a modified oligonucleotide consisting of 20 to 80 linked nucleosides and having a nucleobase sequence comprising the nucleobase sequence of any one of SEQ ID NOs: 511-548 or 589-599. In certain embodiments, the compound is an antisense compound or oligomeric compound. In certain embodiments, the compound is single-stranded. In certain embodiments, the compound is double-stranded. In certain embodiments, the modified oligonucleotide consists of 20 to 30 linked nucleosides.

Certain embodiments provide a compound comprising a modified oligonucleotide having a nucleobase sequence consisting of the nucleobase sequence of any one of SEQ ID NOs: 3-599. In certain embodiments, the compound is an antisense compound or oligomeric compound. In certain embodiments, the compound is single-stranded. In certain embodiments, the compound is double-stranded.

In certain embodiments, any of the foregoing modified oligonucleotides has at least one modified internucleoside linkage, at least one modified sugar, and/or at least one modified nucleobase.

In certain embodiments, at least one nucleoside of any of the foregoing modified oligonucleotides comprises a modified sugar. In certain embodiments, the modified sugar comprises a 2′-O-methoxyethyl group. In certain embodiments, the modified sugar is a bicyclic sugar, such as a 4′-CH(CH₃)—O-2′ group, a 4′-CH₂—O-2′ group, or a 4′-(CH₂)₂—O-2′ group.

In certain embodiments, at least one internucleoside linkage of the modified oligonucleotide comprises a modified internucleoside linkage, such as a phosphorothioate internucleoside linkage.

In certain embodiments, at least one nucleobase of any of the foregoing modified oligonucleotides is a modified nucleobase, such as 5-methylcytosine.

In certain embodiments, any of the foregoing modified oligonucleotides has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 80 linked nucleosides and has a nucleobase sequence comprising the nucleobase sequence of any one of SEQ ID NOs: 3-470 or 549-588. In certain embodiments, the modified oligonucleotide consists of 16 to 30 linked nucleosides and has a nucleobase sequence comprising the nucleobase sequence of any one of SEQ ID NOs: 3-470 or 549-588. In certain embodiments, the modified oligonucleotide consists of 16 linked nucleosides and has a nucleobase sequence consisting of the nucleobase sequence of any one of SEQ ID NOs: 3-470 or 549-588. In certain embodiments, the modified oligonucleotide consists of 18 to 80 linked nucleosides and has a nucleobase sequence comprising the nucleobase sequence of any one of SEQ ID NOs: 471-510. In certain embodiments, the modified oligonucleotide consists of 18 to 30 linked nucleosides and has a nucleobase sequence comprising the nucleobase sequence of any one of SEQ ID NOs: 471-510. In certain embodiments, the modified oligonucleotide consists of 18 linked nucleosides and has a nucleobase sequence consisting of the nucleobase sequence of any one of SEQ ID NOs: 471-510. In certain embodiments, the modified oligonucleotide consists of 20 to 80 linked nucleosides and has a nucleobase sequence comprising the nucleobase sequence of any one of SEQ ID NOs: 511-548 or 589-599. In certain embodiments, the modified oligonucleotide consists of 20 to 30 linked nucleosides and has a nucleobase sequence comprising the nucleobase sequence of any one of SEQ ID NOs: 511-548 or 589-599. In certain embodiments, the modified oligonucleotide consists of 20 linked nucleosides and has a nucleobase sequence consisting of the nucleobase sequence of any one of SEQ ID NOs: 511-548 or 589-599.

In certain embodiments, a compound comprises or consists of a modified oligonucleotide consisting of 16 to 80 linked nucleobases and has a nucleobase sequence comprising the nucleobase sequence of any one of SEQ ID NOs: 3-599, wherein the modified oligonucleotide has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 16 to 80 linked nucleobases and has a nucleobase sequence comprising the nucleobase sequence of any one of SEQ ID NOs: 3-470, wherein the modified oligonucleotide has:

a gap segment consisting of ten linked 2′-deoxynucleosides;

a 5′ wing segment consisting of three linked nucleosides; and

a 3′ wing segment consisting of three linked nucleosides;

wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 18 to 80 linked nucleobases and has a nucleobase sequence comprising the nucleobase sequence of any one of SEQ ID NOs: 471-510, wherein each nucleoside of the modified oligonucleotide comprises a 2′-MOE nucleoside; wherein each internucleoside linkage is a phosphorothioate linkage; and wherein each cytosine is a 5-methylcytosine. In certain embodiments, the modified oligonucleotide consists of 18 to 30 linked nucleosides. In certain embodiments, the modified oligonucleotide consists of 18 linked nucleosides.

In certain embodiments, a compound comprises or consists of a modified oligonucleotide consisting of 20 to 80 linked nucleobases and has a nucleobase sequence comprising the nucleobase sequence of any one of SEQ ID NOs: 511-548 or 589-599, wherein each nucleoside of the modified oligonucleotide comprises a 2′-MOE nucleoside; wherein each internucleoside linkage is a phosphorothioate linkage; and wherein each cytosine is a 5-methylcytosine. In certain embodiments, the modified oligonucleotide consists of 20 to 30 linked nucleosides. In certain embodiments, the modified oligonucleotide consists of 20 linked nucleosides.

In certain embodiments, a compound comprises or consists of a modified oligonucleotide consisting of 16 linked nucleobases and has a nucleobase sequence consisting of the nucleobase sequence of any one of SEQ ID NOs: 549-588, wherein the modified oligonucleotide comprises the sugar motif: kddkddkddkddkddk in the 5′ to 3′ direction, wherein “k” indicates a cEt sugar moiety and “d” indicates an unmodified 2′-deoxyribosyl sugar moiety; wherein each internucleoside linkage is a phosphorothioate linkage; and wherein each cytosine is a 5-methylcytosine.

In certain embodiments, a compound comprises or consists of a modified oligonucleotide consisting of 16 linked nucleobases and has a nucleobase sequence consisting of the nucleobase sequence of any one of SEQ ID NOs: 549-588, wherein the modified oligonucleotide comprises the sugar motif: keekeekeekeekeek in the 5′ to 3′ direction, wherein “k” indicates a cEt sugar moiety and “e” indicates 2′-MOE sugar moiety; wherein each internucleoside linkage is a phosphorothioate linkage; and wherein each cytosine is a 5-methylcytosine.

In any of the foregoing embodiments, the compound or oligonucleotide can be at least 85%, at least 90%, at least 95%, at least 98%, at least 99%, or 100% complementary to a SARS-CoV-2 RNA.

In certain embodiments, the modified oligonucleotide is described by its Compound Number or ION number in the Examples section below.

In any of the foregoing embodiments, the compound can be single-stranded. In certain embodiments, the compound comprises deoxyribonucleotides. In certain embodiments, the compound is double-stranded. In certain embodiments, the compound is double-stranded and comprises ribonucleotides. In any of the foregoing embodiments, the compound can be an antisense compound or oligomeric compound.

In any of the foregoing embodiments, the compound can consist of 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides. In certain embodiments, the compound comprises or consists of an oligonucleotide.

In certain embodiments, a compound is a modified oligonucleotide described by its Compound Number or ION number in the Examples section below.

In certain embodiments, compounds or compositions provided herein comprise a salt of the modified oligonucleotide. In certain embodiments, the salt is a sodium salt. In certain embodiments, the salt is a potassium salt.

### Certain Indications

Certain embodiments provided herein relate to methods of inhibiting or reducing SARS-CoV-2 replication, infectivity, viral titer, or viral load, which can be useful for preventing or treating COVID-19 in an individual, by administration of a compound that targets SARS-CoV-2 RNA. In certain embodiments, the compound can be an antisense compound, oligomeric compound, or oligonucleotide targeted to SARS-CoV-2 RNA.

In certain embodiments, a method of inhibiting or reducing SARS-CoV-2 replication, infectivity, viral titer, or viral load, in lung cells comprises contacting lung cells with a compound comprising a SARS-CoV-2 specific inhibitor, thereby inhibiting or reducing SARS-CoV-2 replication, infectivity, viral titer, or viral load in lung cells. In certain embodiments, the compound comprises an antisense compound targeted to SARS-CoV-2 RNA. In certain embodiments, the compound comprises an oligonucleotide targeted to SARS-CoV-2 RNA. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 3-599. In certain embodiments, a compound comprises a modified oligonucleotide consisting of 16 to 80 linked nucleosides and having a nucleobase sequence comprising the nucleobase sequence of any one of SEQ ID NOs: 3-599. In certain embodiments, a compound comprises a modified oligonucleotide having the nucleobase sequence of any one of SEQ ID NOs: 3-599. In certain embodiments, the modified oligonucleotide consists of 16 to 80 linked nucleosides and has a nucleobase sequence comprising the nucleobase sequence of any one of SEQ ID NOs: 3-470 or 549-588. In certain embodiments, the modified oligonucleotide consists of 16 to 30 linked nucleosides and has a nucleobase sequence comprising the nucleobase sequence of any one of SEQ ID NOs: 3-470 or 549-588. In certain embodiments, the modified oligonucleotide consists of 16 linked nucleosides and has a nucleobase sequence consisting of the nucleobase sequence of any one of SEQ ID NOs: 3-470 or 549-588. In certain embodiments, the modified oligonucleotide consists of 18 to 80 linked nucleosides and has a nucleobase sequence comprising the nucleobase sequence of any one of SEQ ID NOs: 471-510. In certain embodiments, the modified oligonucleotide consists of 18 to 30 linked nucleosides and has a nucleobase sequence comprising the nucleobase sequence of any one of SEQ ID NOs: 471-510. In certain embodiments, the modified oligonucleotide consists of 18 linked nucleosides and has a nucleobase sequence consisting of the nucleobase sequence of any one of SEQ ID NOs: 471-510. In certain embodiments, the modified oligonucleotide consists of 20 to 80 linked nucleosides and has a nucleobase sequence comprising the nucleobase sequence of any one of SEQ ID NOs: 511-548 or 589-599. In certain embodiments, the modified oligonucleotide consists of 20 to 30 linked nucleosides and has a nucleobase sequence comprising the nucleobase sequence of any one of SEQ ID NOs: 511-548 or 589-599. In certain embodiments, the modified oligonucleotide consists of 20 linked nucleosides and has a nucleobase sequence consisting of the nucleobase sequence of any one of SEQ ID NOs: 511-548 or 589-599.

In certain embodiments, a method of inhibiting or reducing SARS-CoV-2 replication, infectivity, viral titer, or viral load, in an individual comprises administering to the individual a compound comprising a SARS-CoV-2 specific inhibitor, thereby preventing or treating COVID-19 in the individual. In certain embodiments, the compound comprises an antisense compound targeted to SARS-CoV-2 RNA. In certain embodiments, the compound comprises an oligonucleotide targeted to SARS-CoV-2 RNA. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 3-599. In certain embodiments, a compound comprises a modified oligonucleotide consisting of 16 to 80 linked nucleosides and having a nucleobase sequence comprising the nucleobase sequence of any one of SEQ ID NOs: 3-599. In certain embodiments, a compound comprises a modified oligonucleotide having the nucleobase sequence of any one of SEQ ID NOs: 3-599. In certain embodiments, the modified oligonucleotide consists of 16 to 80 linked nucleosides and has a nucleobase sequence comprising the nucleobase sequence of any one of SEQ ID NOs: 3-470 or 549-588. In certain embodiments, the modified oligonucleotide consists of 16 to 30 linked nucleosides and has a nucleobase sequence comprising the nucleobase sequence of any one of SEQ ID NOs: 3-470 or 549-588. In certain embodiments, the modified oligonucleotide consists of 16 linked nucleosides and has a nucleobase sequence consisting of the nucleobase sequence of any one of SEQ ID NOs: 3-470 or 549-588. In certain embodiments, the modified oligonucleotide consists of 18 to 80 linked nucleosides and has a nucleobase sequence comprising the nucleobase sequence of any one of SEQ ID NOs: 471-510. In certain embodiments, the modified oligonucleotide consists of 18 to 30 linked nucleosides and has a nucleobase sequence comprising the nucleobase sequence of any one of SEQ ID NOs: 471-510. In certain embodiments, the modified oligonucleotide consists of 18 linked nucleosides and has a nucleobase sequence consisting of the nucleobase sequence of any one of SEQ ID NOs: 471-510. In certain embodiments, the modified oligonucleotide consists of 20 to 80 linked nucleosides and has a nucleobase sequence comprising the nucleobase sequence of any one of SEQ ID NOs: 511-548 or 589-599. In certain embodiments, the modified oligonucleotide consists of 20 to 30 linked nucleosides and has a nucleobase sequence comprising the nucleobase sequence of any one of SEQ ID NOs: 511-548 or 589-599. In certain embodiments, the modified oligonucleotide consists of 20 linked nucleosides and has a nucleobase sequence consisting of the nucleobase sequence of any one of SEQ ID NOs: 511-548 or 589-599.

Certain embodiments are drawn to a compound comprising a SARS-CoV-2 specific inhibitor for use in inhibiting or reducing SARS-CoV-2 replication, infectivity, viral titer, or viral load in lung cells or an individual. Certain embodiments are drawn to a compound comprising a SARS-CoV-2 specific inhibitor for use in preventing or treating COVID-19 in an individual. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 3-599. In certain embodiments, a compound comprises a modified oligonucleotide consisting of 16 to 80 linked nucleosides and having a nucleobase sequence comprising the nucleobase sequence of any one of SEQ ID NOs: 3-599. In certain embodiments, a compound comprises a modified oligonucleotide having the nucleobase sequence of any one of SEQ ID NOs: 3-599. In certain embodiments, the modified oligonucleotide consists of 16 to 80 linked nucleosides and has a nucleobase sequence comprising the nucleobase sequence of any one of SEQ ID NOs: 3-470 or 549-588. In certain embodiments, the modified oligonucleotide consists of 16 to 30 linked nucleosides and has a nucleobase sequence comprising the nucleobase sequence of any one of SEQ ID NOs: 3-470 or 549-588. In certain embodiments, the modified oligonucleotide consists of 16 linked nucleosides and has a nucleobase sequence consisting of the nucleobase sequence of any one of SEQ ID NOs: 3-470 or 549-588. In certain embodiments, the modified oligonucleotide consists of 18 to 80 linked nucleosides and has a nucleobase sequence comprising the nucleobase sequence of any one of SEQ ID NOs: 471-510. In certain embodiments, the modified oligonucleotide consists of 18 to 30 linked nucleosides and has a nucleobase sequence comprising the nucleobase sequence of any one of SEQ ID NOs: 471-510. In certain embodiments, the modified oligonucleotide consists of 18 linked nucleosides and has a nucleobase sequence consisting of the nucleobase sequence of any one of SEQ ID NOs: 471-510. In certain embodiments, the modified oligonucleotide consists of 20 to 80 linked nucleosides and has a nucleobase sequence comprising the nucleobase sequence of any one of SEQ ID NOs: 511-548 or 589-599. In certain embodiments, the modified oligonucleotide consists of 20 to 30 linked nucleosides and has a nucleobase sequence comprising the nucleobase sequence of any one of SEQ ID NOs: 511-548 or 589-599. In certain embodiments, the modified oligonucleotide consists of 20 linked nucleosides and has a nucleobase sequence consisting of the nucleobase sequence of any one of SEQ ID NOs: 511-548 or 589-599.

Certain embodiments are drawn to use of a compound comprising a SARS-CoV-2 specific inhibitor for the manufacture or preparation of a medicament for inhibiting or reducing SARS-CoV-2 replication, infectivity, viral titer, or viral load in lung cells or an individual. Certain embodiments are drawn to a compound comprising a SARS-CoV-2 specific inhibitor for the manufacture or preparation of a medicament for preventing or treating COVID-19 in an individual. In certain embodiments, the compound comprises a modified oligonucleotide consisting of 8 to 80 linked nucleosides and having a nucleobase sequence comprising at least 8 contiguous nucleobases of any of the nucleobase sequences of SEQ ID NOs: 3-599. In certain embodiments, a compound comprises a modified oligonucleotide consisting of 16 to 80 linked nucleosides and having a nucleobase sequence comprising the nucleobase sequence of any one of SEQ ID NOs: 3-599. In certain embodiments, a compound comprises a modified oligonucleotide having the nucleobase sequence of any one of SEQ ID NOs: 3-599. In certain embodiments, the modified oligonucleotide consists of 16 to 80 linked nucleosides and has a nucleobase sequence comprising the nucleobase sequence of any one of SEQ ID NOs: 3-470 or 549-588. In certain embodiments, the modified oligonucleotide consists of 16 to 30 linked nucleosides and has a nucleobase sequence comprising the nucleobase sequence of any one of SEQ ID NOs: 3-470 or 549-588. In certain embodiments, the modified oligonucleotide consists of 16 linked nucleosides and has a nucleobase sequence consisting of the nucleobase sequence of any one of SEQ ID NOs: 3-470 or 549-588. In certain embodiments, the modified oligonucleotide consists of 18 to 80 linked nucleosides and has a nucleobase sequence comprising the nucleobase sequence of any one of SEQ ID NOs: 471-510. In certain embodiments, the modified oligonucleotide consists of 18 to 30 linked nucleosides and has a nucleobase sequence comprising the nucleobase sequence of any one of SEQ ID NOs: 471-510. In certain embodiments, the modified oligonucleotide consists of 18 linked nucleosides and has a nucleobase sequence consisting of the nucleobase sequence of any one of SEQ ID NOs: 471-510. In certain embodiments, the modified oligonucleotide consists of 20 to 80 linked nucleosides and has a nucleobase sequence comprising the nucleobase sequence of any one of SEQ ID NOs: 511-548 or 589-599. In certain embodiments, the modified oligonucleotide consists of 20 to 30 linked nucleosides and has a nucleobase sequence comprising the nucleobase sequence of any one of SEQ ID NOs: 511-548 or 589-599. In certain embodiments, the modified oligonucleotide consists of 20 linked nucleosides and has a nucleobase sequence consisting of the nucleobase sequence of any one of SEQ ID NOs: 511-548 or 589-599.

In any of the foregoing methods or uses, the compound can comprise or consist of a modified oligonucleotide consisting of 16 to 80 linked nucleobases and having a nucleobase sequence comprising the nucleobase sequence of any one of SEQ ID NOs: 3-599, wherein the modified oligonucleotide has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar. In certain embodiments, the modified oligonucleotide consists of 16 to 30 linked nucleosides. In certain embodiments, the modified oligonucleotide consists of 16 linked nucleosides.

In any of the foregoing methods or uses, the compound can comprise or consist of a modified oligonucleotide consisting of 16 to 80 linked nucleobases and having a nucleobase sequence comprising the nucleobase sequence of any one of SEQ ID NOs: 3-470, wherein the modified oligonucleotide has:

a gap segment consisting of ten linked 2′-deoxynucleosides;

a 5′ wing segment consisting of three linked nucleosides; and

a 3′ wing segment consisting of three linked nucleosides;

wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

In any of the foregoing methods or uses, the compound can comprise or consist of a modified oligonucleotide consisting of 18 to 80 linked nucleobases and having a nucleobase sequence comprising the nucleobase sequence of any one of SEQ ID NOs: 471-510, wherein each nucleoside of the modified oligonucleotide comprises a 2′-MOE nucleoside; wherein each internucleoside linkage is a phosphorothioate linkage; and wherein each cytosine is a 5-methylcytosine. In certain embodiments, the modified oligonucleotide consists of 18 to 30 linked nucleosides. In certain embodiments, the modified oligonucleotide consists of 18 linked nucleosides.

In any of the foregoing methods or uses, the compound can comprise or consist of a modified oligonucleotide consisting of 20 to 80 linked nucleobases and having a nucleobase sequence comprising the nucleobase sequence of any one of SEQ ID NOs: 511-548 or 589-599, wherein each nucleoside of the modified oligonucleotide comprises a 2′-MOE nucleoside; wherein each internucleoside linkage is a phosphorothioate linkage; and wherein each cytosine is a 5-methylcytosine. In certain embodiments, the modified oligonucleotide consists of 20 to 30 linked nucleosides. In certain embodiments, the modified oligonucleotide consists of 20 linked nucleosides.

In any of the foregoing methods or uses, the compound can comprise or consist of a modified oligonucleotide consisting of 16 linked nucleobases and having a nucleobase sequence consisting of the nucleobase sequence of any one of SEQ ID NOs: 549-588, wherein the modified oligonucleotide comprises the sugar motif: kddkddkddkddkddk in the 5′ to 3′ direction, wherein “k” indicates a cEt sugar moiety and “d” indicates an unmodified 2′-deoxyribosyl sugar moiety; wherein each internucleoside linkage is a phosphorothioate linkage; and wherein each cytosine is a 5-methylcytosine.

In any of the foregoing methods or uses, the compound can comprise or consist of a modified oligonucleotide consisting of 16 linked nucleobases and having a nucleobase sequence consisting of the nucleobase sequence of any one of SEQ ID NOs: 549-588, wherein the modified oligonucleotide comprises the sugar motif: keekeekeekeekeek in the 5′ to 3′ direction, wherein “k” indicates a cEt sugar moiety and “e” indicates 2′-MOE sugar moiety; wherein each internucleoside linkage is a phosphorothioate linkage; and wherein each cytosine is a 5-methylcytosine.

In any of the foregoing methods or uses, the compound can be targeted to SARS-CoV-2 RNA. In certain embodiments, the compound comprises or consists of a modified oligonucleotide, for example a modified oligonucleotide consisting of 8 to 80 linked nucleosides, 10 to 30 linked nucleosides in length, 12 to 30 linked nucleosides in length, or 20 linked nucleosides in length. In certain embodiments, the modified oligonucleotide is at least 80%, 85%, 90%, 95% or 100% complementary to any of the nucleobase sequences recited in SEQ ID NO: 1 or 2. In certain embodiments, the modified oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing methods or uses, the modified oligonucleotide can be 12 to 30, 15 to 30, 15 to 25, 15 to 24, 16 to 24, 17 to 24, 18 to 24, 19 to 24, 20 to 24, 19 to 22, 20 to 22, 16 to 20, or 17 or 20 linked nucleosides in length. In certain embodiments, the modified oligonucleotide is at least 80%, 85%, 90%, 95% or 100% complementary to any of the nucleobase sequences recited in SEQ ID NO: 1 or 2. In certain embodiments, the modified oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In any of the foregoing methods or uses, the modified oligonucleotide can be one that is described by its Compound Number or ION number in the Examples section below.

In any of the foregoing methods or uses, the compound can be administered in an aerosol form. In any of the foregoing methods or uses, the compound can be administered to the lungs of a patient. In any of the foregoing methods or uses, the compound can be administered by inhalation. In any of the foregoing methods or uses, the compound can be administered by an inhaler. In any of the foregoing methods or uses, the compound can be administered by a nebulizer.

### Certain Numbered Embodiments

The following are certain numbered embodiments that are not limiting on any other embodiments described herein:

Embodiment 1. A compound comprising a modified oligonucleotide consisting of 8 to 80 linked nucleosides and having a nucleobase sequence comprising at least 8 contiguous nucleobases of any of the nucleobase sequences of SEQ ID NOs: 3-599.

Embodiment 2. A compound comprising a modified oligonucleotide consisting of 9 to 80 linked nucleosides and having a nucleobase sequence comprising at least 9 contiguous nucleobases of any of the nucleobase sequences of SEQ ID NOs: 3-599.

Embodiment 3. A compound comprising a modified oligonucleotide consisting of 10 to 80 linked nucleosides and having a nucleobase sequence comprising at least 10 contiguous nucleobases of any of the nucleobase sequences of SEQ ID NOs: 3-599.

Embodiment 4. A compound comprising a modified oligonucleotide consisting of 11 to 80 linked nucleosides and having a nucleobase sequence comprising at least 11 contiguous nucleobases of any of the nucleobase sequences of SEQ ID NOs: 3-599.

Embodiment 5. A compound comprising a modified oligonucleotide consisting of 12 to 80 linked nucleosides and having a nucleobase sequence comprising at least 12 contiguous nucleobases of any of the nucleobase sequences of SEQ ID NOs: 3-599.

Embodiment 6. A compound comprising a modified oligonucleotide consisting of 16 to 80 linked nucleosides and having a nucleobase sequence comprising the nucleobase sequence of any one of SEQ ID NOs: 3-470 or 549-588.

Embodiment 7. A compound comprising a modified oligonucleotide consisting of 18 to 80 linked nucleosides and having a nucleobase sequence comprising the nucleobase sequence of any one of SEQ ID NOs: 471-510.

Embodiment 8. A compound comprising a modified oligonucleotide consisting of 20 to 80 linked nucleosides and having a nucleobase sequence comprising the nucleobase sequence of any one of SEQ ID NOs: 511-548 or 589-599.

Embodiment 9. A compound comprising a modified oligonucleotide having a nucleobase sequence consisting of the nucleobase sequence of any one of SEQ ID NOs: 3-599.

Embodiment 10. The compound of any one of embodiments 1-9, wherein at least one internucleoside linkage of the modified oligonucleotide is a modified internucleoside linkage, at least one nucleoside of the modified oligonucleotide comprises a modified sugar, or at least one nucleobase of the modified oligonucleotide is a modified nucleobase.

Embodiment 11. The compound of embodiment 10, wherein the modified internucleoside linkage is a phosphorothioate internucleoside linkage.

Embodiment 12. The compound of embodiment 10 or 11, wherein the modified sugar is a bicyclic sugar.

Embodiment 13. The compound of embodiment 12, wherein the bicyclic sugar is selected from the group consisting of: 4′-(CH₂)—O-2′ (LNA); 4′-(CH₂)₂—O-2′ (ENA); and 4′-CH(CH₃)—O-2′ (cEt).

Embodiment 14. The compound of embodiment 10 or 11, wherein the modified sugar is 2′-O-methoxyethyl.

Embodiment 15. The compound of any one of embodiments 10-14, wherein the modified nucleobase is a 5-methylcytosine.

Embodiment 16. The compound of any one of embodiments 1-15, wherein the modified oligonucleotide has:
a gap segment consisting of linked 2′-deoxynucleosides;
a 5′ wing segment consisting of linked nucleosides; and
a 3′ wing segment consisting of linked nucleosides;

wherein the gap segment is positioned between the 5′ wing segment and the 3′ wing segment and wherein each nucleoside of each wing segment comprises a modified sugar.

Embodiment 17. A modified oligonucleotide consisting of 16 to 80 linked nucleobases and having a nucleobase sequence comprising the nucleobase sequence of any one of SEQ ID NOs: 3-470, wherein the modified oligonucleotide has:
a gap segment consisting of ten linked 2′-deoxynucleosides;
a 5′ wing segment consisting of three linked nucleosides; and
a 3′ wing segment consisting of three linked nucleosides;

wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine.

Embodiment 18. A modified oligonucleotide consisting of 18 to 80 linked nucleobases and having a nucleobase sequence comprising the nucleobase sequence of any one of SEQ ID NOs: 471-510, wherein each nucleoside of the modified oligonucleotide comprises a 2′-MOE nucleoside; wherein each internucleoside linkage is a phosphorothioate linkage; and wherein each cytosine is a 5-methylcytosine.

Embodiment 19. A modified oligonucleotide consisting of 20 to 80 linked nucleobases and having a nucleobase sequence comprising the nucleobase sequence of any one of SEQ ID NOs: 511-548 or 589-599, wherein each nucleoside of the modified oligonucleotide comprises a 2′-MOE nucleoside; wherein each internucleoside linkage is a phosphorothioate linkage; and wherein each cytosine is a 5-methylcytosine.

Embodiment 20. A modified oligonucleotide consisting of 16 linked nucleobases and having a nucleobase sequence consisting of the nucleobase sequence of any one of SEQ ID NOs: 549-588, wherein the modified oligonucleotide comprises the sugar motif: kddkddkddkddkddk in the 5′ to 3′ direction, wherein wherein “k” indicates a cEt sugar moiety and “d” indicates an unmodified 2′-deoxyribosyl sugar moiety; wherein each internucleoside linkage is a phosphorothioate linkage; and wherein each cytosine is a 5-methylcytosine.

Embodiment 21. A modified oligonucleotide consisting of 16 linked nucleobases and having a nucleobase sequence consisting of the nucleobase sequence of any one of SEQ ID NOs: 549-588, wherein the modified oligonucleotide comprises the sugar motif: keekeekeekeekeek in the 5′ to 3′ direction, wherein wherein “k” indicates a cEt sugar moiety and “e” indicates 2′-MOE sugar moiety; wherein each internucleoside linkage is a phosphorothioate linkage; and wherein each cytosine is a 5-methylcytosine.

Embodiment 22. The compound of any one of embodiments 1-21, wherein the oligonucleotide is at least 80%, 85%, 90%, 95% or 100% complementary to SEQ ID NO:1 or 2.

Embodiment 23. The compound of any one of embodiments 1-22, wherein the compound is single-stranded.

Embodiment 24. The compound of any one of embodiments 1-22, wherein the compound is double-stranded.

Embodiment 25. The compound of any one of embodiments 1-22, wherein the compound comprises ribonucleotides.

Embodiment 26. The compound of any one of embodiments 1-22, wherein the compound comprises deoxyribonucleotides.

Embodiment 27. The compound of any one of embodiments 1-21, wherein the modified oligonucleotide consists of 16 to 30 linked nucleosides or 18 to 30 linked nucleosides, or 20 to 30 linked nucleosides.

Embodiment 28. The compound of any one of embodiments 1-27, wherein the compound consists of the modified oligonucleotide.

Embodiment 29. A compound consisting of a pharmaceutically acceptable salt of any of the compounds of embodiments 1-28.

Embodiment 30. The compound of embodiment 29, wherein the pharmaceutically acceptable salt is a sodium salt.

Embodiment 31. The compound of embodiment 30, wherein the pharmaceutically acceptable salt is a potassium salt.

Embodiment 32. A composition comprising the compound of any one of embodiments 1-31 and a pharmaceutically acceptable diluent or carrier.

Embodiment 33. A composition comprising the compound of any one of embodiments 1-31 and water.

Embodiment 34. A composition comprising a compound of any one of embodiments 1-32, for use in therapy.

Embodiment 35. A method of inhibiting or reducing SARS-CoV-2 replication, infectivity, viral titer, or viral load in lung cells comprising contacting the lung cells with the compound of any one of embodiments 1-31 or composition of any one of embodiments 32-34, thereby inhibiting or reducing SARS-CoV-2 replication, infectivity, viral titer, or viral load in the lung cells.

Embodiment 36. A method of inhibiting or reducing SARS-CoV-2 replication, infectivity, viral titer, or viral load in an individual comprising administering to the individual the compound of any one of embodiments 1-31 or composition of any one of embodiments 32-34, thereby inhibiting or reducing SARS-CoV-2 replication, infectivity, viral titer, or viral load in the individual.

Embodiment 37. A method of preventing or treating COVID-19 in an individual comprising administering to the individual the compound of any one of embodiments 1-31 or composition of any one of embodiments 32-34, thereby preventing or treating COVID-19 in the individual.

Embodiment 38. The method of any of embodiments 35-37, wherein contacting or administering the compound of any one of embodiments 1-31 or composition of any one of embodiments 32-34 prevents or improves a COVID-19 symptom.

Embodiment 39. The method of embodiment 38, wherein the COVID symptom is respiratory illness, difficulty breathing, fever, cough, fatigue, aches and pains, sore throat, runny nose, diarrhea, loss of taste or smell, or nasal congestion.

Embodiment 40. Use of the compound of any one of embodiments 1-31 or composition of any one of embodiments 32-34 for inhibiting or reducing SARS-CoV-2 replication, infectivity, viral titer, or viral load in lung cells.

Embodiment 41. Use of the compound of any one of embodiments 1-31 or composition of any one of embodiments 32-34 for inhibiting or reducing SARS-CoV-2 replication, infectivity, viral titer, or viral load in an individual.

Embodiment 42. Use of the compound of any one of embodiments 1-31 or composition of any one of embodiments 32-34 for preventing or treating COVID-19 in an individual.

Embodiment 43. The use of any of embodiments 40-42, for preventing or improving a COVID-19 symptom.

Embodiment 44. The use of embodiment 43, wherein the COVID symptom is respiratory illness, difficulty breathing, fever, cough, fatigue, aches and pains, sore throat, runny nose, diarrhea, loss of taste or smell, or nasal congestion.

Embodiment 45. Use of the compound of any one of embodiments 1-31 or composition of any one of embodiments 32-34 for the preparation or manufacture of a medicament for inhibiting or reducing SARS-CoV-2 replication, infectivity, viral titer, or viral load in lung cells.

Embodiment 46. Use of the compound of any one of embodiments 1-31 or composition of any one of embodiments 32-34 for the preparation or manufacture of a medicament for inhibiting or reducing SARS-CoV-2 replication, infectivity, viral titer, or viral load in an individual.

Embodiment 47. Use of the compound of any one of embodiments 1-31 or composition of any one of embodiments 32-34 for the preparation or manufacture of a medicament for preventing or treating COVID-19 in an individual.

Embodiment 48. The use of any of embodiments 40-42, for the preparation or manufacture of a medicament for preventing or improving a COVID-19 symptom.

Embodiment 49. The use of embodiment 43, wherein the COVID symptom is respiratory illness, difficulty breathing, fever, cough, fatigue, aches and pains, sore throat, runny nose, diarrhea, loss of taste or smell, or nasal congestion.

### Certain Combinations

In certain embodiments, a first agent comprising a compound described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the first agent to treat an undesired effect of the first agent. In certain embodiments, such second agents are designed to treat an undesired side effect of one or more pharmaceutical compositions as described herein. In certain embodiments, second agents are co-administered with the first agent to produce a combinational effect. In certain embodiments, second agents are co-administered with the first agent to produce a synergistic effect. In certain embodiments, the co-administration of the first and second agents permits use of lower dosages than would be required to achieve a therapeutic or prophylactic effect if the agents were administered as independent therapy.

In certain embodiments, one or more compounds or compositions provided herein are co-administered with one or more secondary agents. In certain embodiments, one or more compounds or compositions provided herein and one or more secondary agents, are administered at different times. In certain embodiments, one or more compounds or compositions provided herein and one or more secondary agents, are prepared together in a single formulation. In certain embodiments, one or more compounds or compositions provided herein and one or more secondary agents, are prepared separately. In certain embodiments, a secondary agent can be one or more of the following: remdesivir, hydroxychloroquine, chloroquine, azithromycin, and/or ivermectin.

### Certain Compounds

In certain embodiments, compounds described herein can be antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide and optionally a conjugate group. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, one of the modified oligonucleotides is conjugated. In certain embodiments, both the modified oligonucleotides are conjugated. In certain embodiments, the first modified oligonucleotide is conjugated. In certain embodiments, the second modified oligonucleotide is conjugated. In certain embodiments, the first modified oligonucleotide is 12-30 linked nucleosides in length and the second modified oligonucleotide is 12-30 linked nucleosides in length. In certain embodiments, one of the modified oligonucleotides has a nucleobase sequence comprising at least 8 contiguous nucleobases of any of SEQ ID NOs: 3-599.

In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, a compound described herein comprises an oligonucleotide 12 to 30 linked subunits in length. In certain embodiments, a compound described herein comprises an oligonucleotide 12 to 22 linked subunits in length. In certain embodiments, compound described herein comprises an oligonucleotide 14 to 30 linked subunits in length. In certain embodiments, compound described herein comprises an oligonucleotide 14 to 20 linked subunits in length. In certain embodiments, a compound described herein comprises an oligonucleotide 15 to 30 linked subunits in length. In certain embodiments, a compound described herein comprises an oligonucleotide 15 to 20 linked subunits in length. In certain embodiments, a compound described herein comprises an oligonucleotide 16 to 30 linked subunits in length. In certain embodiments, a compound described herein comprises an oligonucleotide 16 to 20 linked subunits in length. In certain embodiments, a compound described herein comprises an oligonucleotide 17 to 30 linked subunits in length. In certain embodiments, a compound described herein comprises an oligonucleotide 17 to 20 linked subunits in length. In certain embodiments, a compound described herein comprises an oligonucleotide 18 to 30 linked subunits in length. In certain embodiments, a compound described herein comprises an oligonucleotide 18 to 21 linked subunits in length. In certain embodiments, a compound described herein comprises an oligonucleotide 18 to 20 linked subunits in length. In certain embodiments, a compound described herein comprises an oligonucleotide 20 to 30 linked subunits in length. In other words, such oligonucleotides are 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits in length, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

In certain embodiments, the compound may further comprise additional features or elements, such as a conjugate group, that are attached to the oligonucleotide. In certain embodiments, such compounds are antisense compounds. In certain embodiments, such compounds are oligomeric compounds. In embodiments where a conjugate group comprises a nucleoside (i.e. a nucleoside that links the conjugate group to the oligonucleotide), the nucleoside of the conjugate group is not counted in the length of the oligonucleotide.

In certain embodiments, compounds may be shortened or truncated. For example, a single subunit may be deleted from the 5′ end (5′ truncation), or alternatively from the 3′ end (3′ truncation). A shortened or truncated compound targeted to an SARS-CoV-2 RNA may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. Proc. Natl. Acad. Sci. USA 1992, 89:7305-7309; Gautschi et al. J. Natl. Cancer Inst. March 2001, 93:463-471; Maher and Dolnick Nuc. Acid. Res. 1998, 16:3341-3358). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term “RNAi” is meant to be equivalent to other terms used to describe sequence specific RNA interference, such as post transcriptional gene silencing, translational inhibition, or epigenetics.

In certain embodiments, a compound described herein can comprise any of the oligonucleotide sequences targeted to SARS-CoV-2 RNA described herein. In certain embodiments, the compound can be double-stranded. In certain embodiments, the compound comprises a first strand comprising at least an 8, 9, 10, 11, 12, 13, 14, 15, 16, 17, 18, 19, or 20 contiguous nucleobase portion of any one of SEQ ID NOs: 3-599 and a second strand. In certain embodiments, the compound comprises a first strand comprising the nucleobase sequence of any one of SEQ ID NOs: 3-599 and a second strand. In certain embodiments, the compound comprises ribonucleotides in which the first strand has uracil (U) in place of thymine (T) in any one of SEQ ID NOs: 3-599. In certain embodiments, the compound comprises (i) a first strand comprising a nucleobase sequence complementary to the site on SARS-CoV-2 RNA to which any of SEQ ID NOs: 3-599 is targeted, and (ii) a second strand. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains one or two capped strands, as disclosed, for example, by WO 00/63364, filed Apr. 19, 2000.

In certain embodiments, the first strand of the compound is an siRNA guide strand and the second strand of the compound is an siRNA passenger strand. In certain embodiments, the second strand of the compound is complementary to the first strand. In certain embodiments, each strand of the compound is 16, 17, 18, 19, 20, 21, 22, or 23 linked nucleosides in length. In certain embodiments, the first or second strand of the compound can comprise a conjugate group.

In certain embodiments, a compound described herein can comprise any of the oligonucleotide sequences targeted to SARS-CoV-2 RNA described herein. In certain embodiments, the compound is single stranded. In certain embodiments, such a compound is a single-stranded RNAi (ssRNAi) compound. In certain embodiments, the compound comprises at least an 8, 9, 10, 11, 12, 13, 14, 15, 16, 17, 18, 19, or 20 contiguous nucleobase portion of any one of SEQ ID NOs: 3-599. In certain embodiments, the compound comprises the nucleobase sequence of any one of SEQ ID NOs: 3-599. In certain embodiments, the compound comprises ribonucleotides in which uracil (U) is in place of thymine (T) in any one of SEQ ID NOs: 3-599. In certain embodiments, the compound comprises a nucleobase sequence complementary to the site on SARS-CoV-2 RNA to which any of SEQ ID NOs: 3-599 is targeted. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains a capped strand, as disclosed, for example, by WO 00/63364, filed Apr. 19, 2000. In certain embodiments, the compound consists of 16, 17, 18, 19, 20, 21, 22, or 23 linked nucleosides. In certain embodiments, the compound can comprise a conjugate group.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the compound that are beneficial for use as a therapeutic or research tool. In certain embodiments, radioactive isotopic substitutions may make the compound suitable for research or diagnostic purposes, such as an imaging assay.

### Certain Mechanisms

In certain embodiments, compounds described herein comprise or consist of modified oligonucleotides. In certain embodiments, compounds described herein are antisense compounds. In certain embodiments, compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

Target Nucleic Acids. Target Regions and Nucleotide Sequences

In certain embodiments, compounds described herein comprise or consist of an oligonucleotide comprising a region that is complementary to a SARS-CoV-2 RNA sequence.

SARS-CoV-2 RNA sequences include, without limitation, the following Genbank Accession Nos., each of which is incorporated by reference in its entirety: NC_045512.2 (designated herein as SEQ ID NO: 1); the complement of NC_045512.2 (designated herein as SEQ ID NO: 2); NC_045512, MT350234, MT350236, MT350237, MT350238, MT350239, MT350240, MT350241, MT350242, MT350243, MT350244, MT350245, MT350246, MT350247, MT350248, MT350249, MT350250, MT350251, MT350252, MT350253, MT350254, MT350255, MT350256, MT350257, MT350263, MT350264, MT350265, MT350266, MT350267, MT350268, MT350269, MT350270, MT350271, MT350272, MT350273, MT350274, MT350275, MT350276, MT350277, MT350278, MT350279, MT350280, MT350282, MT344135, MT344944, MT344945, MT344946, MT344947, MT344948, MT344949, MT344950, MT344951, MT344952, MT344953, MT344954, MT344955, MT344956, MT344957, MT344958, MT344959, MT344960, MT344961, MT344962, MT344963, MT345798, MT345799, MT345800, MT345801, MT345802, MT345803, MT345804, MT345805, MT345806, MT345807, MT345808, MT345809, MT345810, MT345811, MT345812, MT345813, MT345814, MT345815, MT345816, MT345817, MT345818, MT345819, MT345820, MT345821, MT345822, MT345823, MT345824, MT345825, MT345826, MT345827, MT345828, MT345829, MT345830, MT345831, MT345832, MT345833, MT345834, MT345835, MT345836, MT345837, MT345838, MT345839, MT345840, MT345841, MT345842, MT345843, MT345844, MT345845, MT345846, MT345847, MT345848, MT345849, MT345850, MT345851, MT345852, MT345853, MT345854, MT345855, MT345856, MT345857, MT345858, MT345859, MT345860, MT345861, MT345862, MT345863, MT345864, MT345865, MT345866, MT345867, MT345868, MT345869, MT345870, MT345871, MT345872, MT345873, MT345874, MT345875, MT345876, MT345877, MT345878, MT345879, MT345880, MT345881, MT345882, MT345883, MT345884, MT345885, MT345886, MT345887, MT345888, MT339039, MT339040, MT339041, MT334522, MT334523, MT334524, MT334525, MT334526, MT334527, MT334528, MT334529, MT334530, MT334531, MT334532, MT334533, MT334534, MT334535, MT334536, MT334537, MT334538, MT334539, MT334540, MT334541, MT334542, MT334543, MT334544, MT334545, MT334546, MT334547, MT334548, MT334549, MT334550, MT334551, MT334552, MT334553, MT334554, MT334555, MT334556, MT334557, MT334558, MT334559, MT334560, MT334561, MT334562, MT334563, MT334564, MT334565, MT334566, MT334567, MT334568, MT334569, MT334570, MT334571, MT334572, MT334573, MT324062, MT324679, MT324680, MT324681, MT324682, MT324683, MT324684, MT325561, MT325562, MT325563, MT325564, MT325565, MT325566, MT325567, MT325568, MT325569, MT325570, MT325571, MT325572, MT325573, MT325574, MT325575, MT325576, MT325577, MT325578, MT325579, MT325580, MT325581, MT325582, MT325583, MT325584, MT325585, MT325586, MT325587, MT325588, MT325589, MT325590, MT325591, MT325592, MT325593, MT325594, MT325595, MT325596, MT325597, MT325598, MT325599, MT325600, MT325601, MT325602, MT325603, MT325604, MT325605, MT325606, MT325607, MT325608, MT325609, MT325610, MT325611, MT325612, MT325613, MT325614, MT325615, MT325616, MT325617, MT325618, MT325619, MT325620, MT325621, MT325622, MT325623, MT325624, MT325625, MT325626, MT325627, MT325628, MT325629, MT325630, MT325631, MT325632, MT325633, MT325634, MT325635, MT325636, MT325637, MT325638, MT325639, MT325640, MT326023, MT326024, MT326025, MT326026, MT326027, MT326028, MT326029, MT326030, MT326031, MT326032, MT326033, MT326034, MT326035, MT326036, MT326037, MT326038, MT326039, MT326040, MT326041, MT326042, MT326043, MT326044, MT326045, MT326046, MT326047, MT326048, MT326049, MT326050, MT326051, MT326052, MT326053, MT326054, MT326055, MT326056, MT326057, MT326058, MT326059, MT326060, MT326061, MT326062, MT326063, MT326064, MT326065, MT326066, MT326067, MT326068, MT326069, MT326070, MT326071, MT326072, MT326073, MT326074, MT326075, MT326076, MT326077, MT326078, MT326079, MT326080, MT326081, MT326082, MT326083, MT326084, MT326085, MT326086, MT326087, MT326088, MT326089, MT326090, MT326091, MT326092, MT326093, MT326094, MT326095, MT326096, MT326097, MT326098, MT326099, MT326100, MT326101, MT326102, MT326103, MT326104, MT326105, MT326106, MT326107, MT326108, MT326109, MT326110, MT326111, MT326112, MT326113, MT326114, MT326115, MT326116, MT326117, MT326118, MT326119, MT326120, MT326121, MT326122, MT326123, MT326124, MT326125, MT326126, MT326127, MT326128, MT326129, MT326130, MT326131, MT326132, MT326133, MT326134, MT326135, MT326136, MT326137, MT326138, MT326139, MT326140, MT326141, MT326142, MT326143, MT326144, MT326145, MT326146, MT326147, MT326148, MT326149, MT326150, MT326151, MT326152, MT326153, MT326154, MT326155, MT326156, MT326157, MT326158, MT326159, MT326160, MT326161, MT326162, MT326163, MT326164, MT326165, MT326166, MT326167, MT326168, MT326169, MT326170, MT326171, MT326172, MT326173, MT326174, MT326175, MT326176, MT326177, MT326178, MT326179, MT326180, MT326181, MT326182, MT326183, MT326184, MT326185, MT326186, MT326187, MT326188, MT326189, MT326190, MT326191, MT327745, MT328032, MT328033, MT328034, MT328035, MT039874, MT077125, MT322394, MT322395, MT322396, MT322397, MT322398, MT322399, MT322400, MT322401, MT322402, MT322403, MT322404, MT322405, MT322406, MT322407, MT322408, MT322409, MT322410, MT322411, MT322412, MT322413, MT322414, MT322415, MT322416, MT322417, MT322418, MT322419, MT322420, MT322421, MT322422, MT322423, MT322424, MT320538, MT320891, MT308692, MT308693, MT308694, MT308695, MT308696, MT308697, MT308698, MT308699, MT308700, MT308701, MT308702, MT308703, MT308704, MT293547, MT300186, MT304474, MT304475, MT304476, MT304477, MT304478, MT304479, MT304480, MT304481, MT304482, MT304483, MT304484, MT304485, MT304486, MT304487, MT304488, MT304489, MT304490, MT304491, MT273658, MT281530, MT281577, MT291826, MT291827, MT291828, MT291829, MT291830, MT291831, MT291832, MT291833, MT291834, MT291835, MT291836, MT292569, MT292570, MT292571, MT292572, MT292573, MT292574, MT292575, MT292576, MT292577, MT292578, MT292579, MT292580, MT292581, MT292582, MT293156, MT293157, MT293158, MT293159, MT293160, MT293161, MT293162, MT293163, MT293164, MT293165, MT293166, MT293167, MT293168, MT293169, MT293170, MT293171, MT293172, MT293173, MT293174, MT293175, MT293176, MT293177, MT293178, MT293179, MT293180, MT293181, MT293182, MT293183, MT293184, MT293185, MT293186, MT293187, MT293188, MT293189, MT293190, MT293191, MT293192, MT293193, MT293194, MT293195, MT293196, MT293197, MT293198, MT293199, MT293200, MT293201, MT293202, MT293203, MT293204, MT293205, MT293206, MT293207, MT293208, MT293209, MT293210, MT293211, MT293212, MT293213, MT293214, MT293215, MT293216, MT293217, MT293218, MT293219, MT293220, MT293221, MT293222, MT293223, MT293224, MT293225, MT295464, MT295465, MT276323, MT276324, MT276325, MT276326, MT276327, MT276328, MT276329, MT276330, MT276331, MT276597, MT276598, MT262896, MT262897, MT262898, MT262899, MT262900, MT262901, MT262902, MT262903, MT262904, MT262905, MT262906, MT262907, MT262908, MT262909, MT262910, MT262911, MT262912, MT262913, MT262914, MT262915, MT262916, MT262993, MT263074, MT263381, MT263382, MT263383, MT263384, MT263385, MT263386, MT263387, MT263388, MT263389, MT263390, MT263391, MT263392, MT263393, MT263394, MT263395, MT263396, MT263397, MT263398, MT263399, MT263400, MT263401, MT263402, MT263403, MT263404, MT263405, MT263406, MT263407, MT263408, MT263409, MT263410, MT263411, MT263412, MT263413, MT263414, MT263415, MT263416, MT263417, MT263418, MT263419, MT263420, MT263421, MT263422, MT263423, MT263424, MT263425, MT263426, MT263427, MT263428, MT263429, MT263430, MT263431, MT263432, MT263433, MT263434, MT263435, MT263436, MT263437, MT263438, MT263439, MT263440, MT263441, MT263442, MT263443, MT263444, MT263445, MT263446, MT263447, MT263448, MT263449, MT263450, MT263451, MT263452, MT263453, MT263454, MT263455, MT263456, MT263457, MT263458, MT263459, MT263460, MT263461, MT263462, MT263463, MT263464, MT263465, MT263466, MT263467, MT263468, MT263469, MT256917, MT256918, MT256924, MT258377, MT258378, MT258379, MT258380, MT258381, MT258382, MT258383, MT259226, MT259227, MT259228, MT259229, MT259230, MT259231, MT259235, MT259236, MT259237, MT259238, MT259239, MT259240, MT259241, MT259242, MT259243, MT259244, MT259245, MT259246, MT259247, MT259248, MT259249, MT259250, MT259251, MT259252, MT259253, MT259254, MT259255, MT259256, MT259257, MT259258, MT259259, MT259260, MT259261, MT259262, MT259263, MT259264, MT259265, MT259266, MT259267, MT259268, MT259269, MT259270, MT259271, MT259272, MT259273, MT259274, MT259275, MT259276, MT259277, MT259278, MT259279, MT259280, MT259281, MT259282, MT259283, MT259284, MT259285, MT259286, MT259287, LC534418, LC534419, MT251972, MT251973, MT251974, MT251975, MT251976, MT251977, MT251978, MT251979, MT251980, MT253696, MT253697, MT253698, MT253699, MT253700, MT253701, MT253702, MT253703, MT253704, MT253705, MT253706, MT253707, MT253708, MT253709, MT253710, MT233526, MT246449, MT246450, MT246451, MT246452, MT246453, MT246454, MT246455, MT246456, MT246457, MT246458, MT246459, MT246460, MT246461, MT246462, MT246463, MT246464, MT246465, MT246466, MT246467, MT246468, MT246469, MT246470, MT246471, MT246472, MT246473, MT246474, MT246475, MT246476, MT246477, MT246478, MT246479, MT246480, MT246481, MT246482, MT246483, MT246484, MT246485, MT246486, MT246487, MT246488, MT246489, MT246490, MT246667, MT240479, MT232869, MT232870, MT232871, MT232872, MT233519, MT233520, MT233521, MT233522, MT233523, MT226610, MT198651, MT198652, MT198653, MT192758, MT192759, MT192765, MT192772, MT192773, MT186676, MT186677, MT186678, MT186679, MT186680, MT186681, MT186682, MT187977, MT188339, MT188340, MT188341, CADDYA000000000, MT184907, MT184908, MT184909, MT184910, MT184911, MT184912, MT184913, MT163712, MT163714, MT163715, MT163716, MT163717, MT163718, MT163719, MT163720, MT163721, MT163737, MT163738, MT066156, MT121215, MT159705, MT159706, MT159707, MT159708, MT159709, MT159710, MT159711, MT159712, MT159713, MT159714, MT159715, MT159716, MT159717, MT159718, MT159719, MT159720, MT159721, MT159722, MT159778, MT161607, LC529905, MT012098, MT050493, MT152824, MT152900, MT135041, MT135042, MT135043, MT135044, MT126808, MT127113, MT127114, MT127115, MT127116, LC528232, LC528233, MT123290, MT123291, MT123292, MT123293, MT118835, MT111895, MT111896, MT106052, MT106053, MT106054, MT093571, MT093631, MT081059, MT081060, MT081061, MT081062, MT081063, MT081064, MT081065, MT081066, MT081067, MT081068, MT072667, MT072668, MT072688, MT066157, MT066158, MT066159, MT066175, MT066176, LC523807, LC523808, LC523809, MT042773, MT042774, MT042775, MT042776, MT042777, MT042778, MT044257, MT044258, MT049951, MT050414, MT050415, MT050416, MT050417, MT039873, MT039887, MT039888, MT039890, LC522350, MT027062, MT027063, MT027064, MT019529, MT019530, MT019531, MT019532, MT019533, MT020781, MT020880, MT020881, LR757995, LR757996, LR757997, LR757998, MT007544, MT008022, MT008023, MN996527, MN996528, MN996529, MN996530, MN996531, MN988668, MN988669, MN994467, MN994468, MN997409, MN988713, MN938384, MN938385, MN938386, MN938387, MN938388, MN938389, MN938390, MN975262, MN975263, MN975264, MN975265, MN975266, MN975267, MN975268, MN985325, MN970003, MN970004, or MN908947.

In certain embodiments, compounds described herein comprise or consist of an oligonucleotide comprising a region that is complementary to a SARS-CoV-2 RNA sequence, wherein the SARS-CoV2 RNA sequence is the B.1.1.7 variant identified in the United Kingdom. In certain embodiments, the RNA sequence of the B.1.1.7 variant is Genbank Accession No. MW487270.

In certain embodiments, compounds described herein comprise or consist of an oligonucleotide comprising a region that is complementary to a SARS-CoV-2 RNA sequence, wherein the SARS-CoV2 RNA sequence is the B.1.351 variant identified in South Africa.

In certain embodiments, compounds described herein comprise or consist of an oligonucleotide comprising a region that is complementary to a SARS-CoV-2 RNA sequence, wherein the SARS-CoV2 RNA sequence is the P.1 variant identified in Brazil.

In certain embodiments, compounds described herein comprise or consist of an oligonucleotide comprising a region that is complementary to a SARS-CoV-2 RNA sequence, wherein the SARS-CoV2 RNA sequence is the B.1.427/B.1.429 variant identified in California.

### Hybridization

In some embodiments, hybridization occurs between a compound disclosed herein and a SARS-CoV-2 RNA. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a SARS-CoV-2 RNA.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a SARS-CoV-2 RNA may be tolerated provided that the compound remains able to specifically hybridize to a target nucleic acid. Moreover, a compound may hybridize over one or more segments of a SARS-CoV-2 RNA such that intervening or adjacent segments are not involved in the hybridization event (e.g., a loop structure, mismatch or hairpin structure).

In certain embodiments, the compounds provided herein, or a specified portion thereof, are, are at least, or are up to 70%, 80%, 85%, 86%, 87%, 88%, 89%, 90%, 91%, 92%, 93%, 94%, 95%, 96%, 97%, 98%, 99%, or 100% complementary to a SARS-CoV-2 RNA, a target region, target segment, or specified portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to a SARS-CoV-2 RNA, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a SARS-CoV-2 RNA, or a target region, or a target segment or target sequence thereof. As used herein, “fully complementary” means each nucleobase of a compound is complementary to the corresponding nucleobase of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a SARS-CoV-2 RNA, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a SARS-CoV-2 RNA, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds, are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific ION number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific ION number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 1, 2, 3, 4, 5, 6, 7, 8, 9, 10, 11, or 12 from the 3′-end of the oligonucleotide.

In certain embodiments, compounds described herein comprise or consist of antisense compounds. In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein comprise or consist of oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Modified Nucleosides

Modified nucleosides comprise a modified sugar moiety or a modified nucleobase or both a modified sugar moiety and a modified nucleobase.

1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₘ) or OCH₂C(═O)—N(Rₘ)(Rₙ), where each Rₘ and Rₙ is, independently, H, an amino protecting group, or substituted or unsubstituted C₁-C₁₀alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearly non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., US2010/190837 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NR)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs. 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

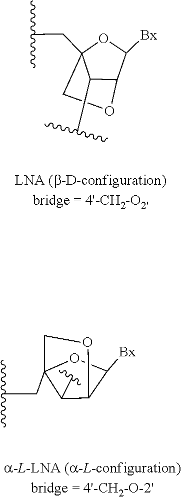

α-L-methyleneoxy (4′-CH₂—O—2′) or α-L-LNA bicyclic nucleosides have been incorporated into oligonucleotides that showed antisense activity (Frieden et al., Nucleic Acids Research. 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Boorg. & Med. Chem. 2002, 10, 841-854), fluoro HNA:

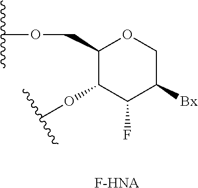

(“F-HNA”, see e.g., Swayze et al., U.S. Pat. No. 8,088,904; Swayze et al., U.S. Pat. No. 8,440,803; and Swayze et al., U.S. Pat. No. 9,005,906, F-HNA can also be referred to as a F-THP or 3′-fluoro tetrahydropyran), and nucleosides comprising additional modified THP compounds having the formula:

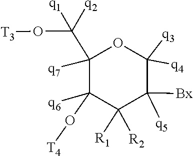

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

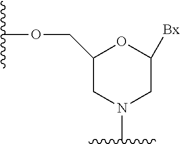

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378.

Many other bicyclic and tricyclic sugar and sugar surrogate ring systems are known in the art that can be used in modified nucleosides.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi-dines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30,613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In certain embodiments, compounds targeted to a SARS-CoV-2 RNA comprise one or more modified nucleobases. In certain embodiments, the modified nucleobase is 5-methylcytosine. In certain embodiments, each cytosine is a 5-methylcytosine.

3. Modified Internucleoside Linkages

The naturally occurring internucleoside linkage of RNA and DNA is a 3′ to 5′ phosphodiester linkage In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

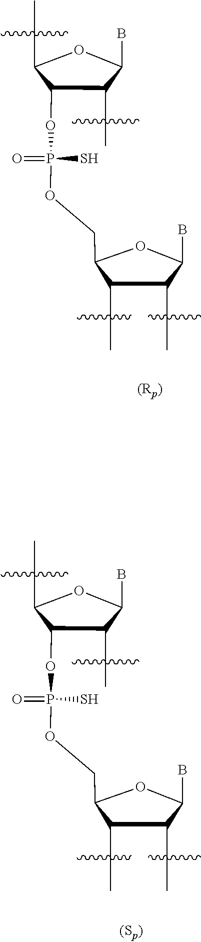

Unless otherwise indicated, chiral internucleoside linkages of modified oligonucleotides described herein can be stereorandom or in a particular stereochemical configuration.

In certain embodiments, compounds targeted to an SARS-CoV-2 RNA comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH2-N(CH3)-O—CH2-), thiodiester, thionocarbamate (—O—C(═O)(NH)—S—); siloxane (—O—SiH2-O—); and N,N′-dimethylhydrazine (—CH2-N(CH3)-N(CH3)-). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)-O-5′), amide-3 (3′-CH2-C(═O)—N(H)-5′), amide-4 (3′-CH2-N(H)—C(═O)-5′), formacetal (3′-O—CH2-O-5′), methoxypropyl, and thioformacetal (3′-S—CH2-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

a. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

In certain embodiments, a modified oligonucleotide can comprise a sugar motif described in Swayze et al., US2010/0197762; Freier et al., US2014/0107330; Freier et al., US2015/0184153; and Seth et al., US2015/0267195, each of which is incorporated by reference in its entirety herein.

Certain embodiments provided herein are directed to modified oligomeric compounds useful for inhibiting target nucleic acid expression, which can be useful for treating, preventing, ameliorating, or slowing progression of a disease associated with such a target nucleic acid. In certain embodiments, the modified oligomeric compounds comprise antisense oligonucleotides that are gapmers having certain sugar motifs. In certain embodiments, the gapmer sugar motifs provided herein can be combined with any nucleobase sequence and any internucleoside linkage motif to form potent antisense oligonucleotides.

b. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

c. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

4. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

Certain Conjugated Compounds

In certain embodiments, the compounds described herein comprise or consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In certain embodiments, the oligonucleotide is modified. In certain embodiments, the oligonucleotide of a compound has a nucleobase sequence that is complementary to a target nucleic acid. In certain embodiments, oligonucleotides are complementary to a messenger RNA (mRNA). In certain embodiments, oligonucleotides are complementary to a sense transcript.

Examples of terminal groups include but are not limited to conjugate groups, capping groups, phosphate moieties, protecting groups, modified or unmodified nucleosides, and two or more nucleosides that are independently modified or unmodified.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compound and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to SARS-CoV-2 RNA can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to SARS-CoV-2 RNA and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### EXAMPLES

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Design of Modified Oligonucleotides Complementary to a SARS-CoV-2 Nucleic Acid

Modified oligonucleotides were designed as indicated in the tables below. The modified oligonucleotides are all 3-10-3 cEt gapmers (i.e., they have a central gap segment of ten 2′-deoxynucleosides flanked on each side by wing segments, each comprising three cEt modified nucleosides). The internucleoside linkages throughout each modified oligonucleotide are phosphorothioate linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are 100% complementary to the genomic sequence of severe acute respiratory syndrome coronavirus 2 isolate Wuhan-Hu-1, designated herein as SEQ ID NO: 1 (GENBANK Accession No. NC_045512.2).

TABLE 1
Design of 3-10-3 cEt modified oligonucleotides
complementary to severe acute respiratory
syndrome coronavirus 2 isolate Wuhan-Hu-1

| | SEQ | SEQ | | |
|---|---|---|---|---|
| | ID | ID | | |
| | NO: | NO: | | |
| | 1 | 1 | | SEQ |
| Compound | Start | Stop | Sequence | ID |
| Number | Site | Site | (5' to 3') | NO |
| 1495586 | 191 | 206 | GACGAAACCGTAAGCA | 3 |
| 1495640 | 26344 | 26359 | ATCGAAGCGCAGTAAG | 4 |
| 1495503 | 13485 | 13500 | TGTAAGACGGGCTGCA | 5 |
| 1495577 | 17141 | 17156 | GTATACACTATGCGAG | 6 |
| 1495616 | 26248 | 26263 | CCGAAACGAATGAGTA | 7 |
| 1495617 | 26249 | 26264 | TCCGAAACGAATGAGT | 8 |
| 1495623 | 26284 | 26299 | GAAGTACGCTATTAAC | 9 |
| 1495672 | 29242 | 29257 | TCCCGAAGGTGTGACT | 10 |
| 1495615 | 26247 | 26262 | CGAAACGAATGAGTAC | 11 |
| 1495642 | 26349 | 26364 | ACACAATCGAAGCGCA | 12 |
| 1495668 | 28521 | 28536 | CGGTAGTAGCCAATTT | 13 |
| 1495701 | 635 | 650 | TATTACCGTTCTTACG | 14 |
| 1495576 | 17140 | 17155 | TATACACTATGCGAGC | 15 |
| 1495597 | 237 | 252 | CACACCCGGACGAAAC | 16 |
| 1495607 | 25509 | 25524 | TCCGAAAGGGAGTGAG | 17 |
| 1495641 | 26348 | 26363 | CACAATCGAAGCGCAG | 18 |
| 1495673 | 29243 | 29258 | TTCCCGAAGGTGTGAC | 19 |
| 1495703 | 6467 | 6482 | CTACAACTTCGGTAGT | 20 |
| 1495527 | 15010 | 15025 | CGAAAAGTGCATCTTG | 21 |
| 1495528 | 15011 | 15026 | GCGAAAAGTGCATCTT | 22 |
| 1495665 | 28417 | 28432 | GAACCAAGACGCAGTA | 23 |
| 1495478 | 11435 | 11450 | GATCTAAAGCATTACC | 24 |
| 1495497 | 13371 | 13386 | CCGCAGACGGTACAGA | 25 |
| 1495500 | 13458 | 13473 | AACCCGTTTAAAAACG | 26 |
| 1495513 | 14667 | 14682 | ATTACCGGGTTTGACA | 27 |
| 1495575 | 17137 | 17152 | ACACTATGCGAGCAGA | 28 |
| 1495585 | 190 | 205 | ACGAAACCGTAAGCAG | 29 |
| 1495608 | 25510 | 25525 | ATCCGAAAGGGAGTGA | 30 |
| 1495483 | 12841 | 12856 | AGGGAATCTAGCCCAT | 31 |
| 1495514 | 14668 | 14683 | AATTACCGGGTTTGAC | 32 |
| 1495639 | 26338 | 26353 | GCGCAGTAAGGATGGC | 33 |
| 1495663 | 28415 | 28430 | ACCAAGACGCAGTATT | 34 |
| 1495664 | 28416 | 28431 | AACCAAGACGCAGTAT | 35 |
| 1495670 | 28812 | 28827 | CGAGAAGAGGCTTGAC | 36 |
| 1495671 | 29241 | 29256 | CCCGAAGGTGTGACTT | 37 |
| 1495613 | 26206 | 26221 | CTTACAAAGGCACGCT | 38 |
| 1495622 | 26283 | 26298 | AAGTACGCTATTAACT | 39 |
| 1495515 | 14669 | 14684 | AAATTACCGGGTTTGA | 40 |
| 1495566 | 15991 | 16006 | TCATAAGTGTACCATC | 41 |
| 1495612 | 26205 | 26220 | TTACAAAGGCACGCTA | 42 |
| 1495619 | 26278 | 26293 | CGCTATTAACTATTAA | 43 |
| 1495646 | 26374 | 26389 | CGTTAACAATATTGCA | 44 |
| 1495518 | 14672 | 14687 | TTAAAATTACCGGGTT | 45 |
| 1495558 | 15846 | 15861 | TTTAGTAAGGTCAGTC | 46 |
| 1495564 | 15889 | 15904 | GTTTAACTAGCATTGT | 47 |
| 1495632 | 26307 | 26322 | GAATACCACGAAAGCA | 48 |
| 1495662 | 28409 | 28424 | ACGCAGTATTATTGGG | 49 |
| 1495679 | 29617 | 29632 | GTTACGAGAATTCATT | 50 |
| 1495688 | 29781 | 29796 | TCCATATAGGCAGCTC | 51 |
| 1495702 | 6464 | 6479 | CAACTTCGGTAGTTTT | 52 |
| 1495517 | 14671 | 14686 | TAAAATTACCGGGTTT | 53 |
| 1495534 | 15074 | 15089 | GCACTAATGGCATACT | 54 |
| 1495560 | 15848 | 15863 | CCTTTAGTAAGGTCAG | 55 |
| 1495625 | 26287 | 26302 | AAAGAAGTACGCTATT | 56 |
| 1495676 | 29613 | 29628 | CGAGAATTCATTCTGC | 57 |
| 1495681 | 29619 | 29634 | TAGTTACGAGAATTCA | 58 |
| 1495690 | 29794 | 29809 | ACATTAGGGCTCTTCC | 59 |
| 1495546 | 15500 | 15515 | CTATTAGCATAAGCAG | 60 |
| 1495548 | 15502 | 15517 | CACTATTAGCATAAGC | 61 |
| 1495611 | 26204 | 26219 | TACAAAGGCACGCTAG | 62 |
| 1495624 | 26286 | 26301 | AAGAAGTACGCTATTA | 63 |
| 1495529 | 15043 | 15058 | TTATAGTAGGGATGAC | 64 |
| 1495531 | 15046 | 15061 | GAGTTATAGTAGGGAT | 65 |
| 1495543 | 15283 | 15298 | GATAATCCCAACCCAT | 66 |
| 1495556 | 15844 | 15859 | TAGTAAGGTCAGTCTC | 67 |
| 1495565 | 15990 | 16005 | CATAAGTGTACCATCT | 68 |
| 1495594 | 20946 | 20961 | ATTAAGATCTGAATCG | 69 |
| 1495620 | 26279 | 26294 | ACGCTATTAACTATTA | 70 |
| 1495628 | 26290 | 26305 | GAAAAAGAAGTACGCT | 71 |
| 1495649 | 26579 | 26594 | TATTACTAGGTTCCAT | 72 |
| 1495697 | 458 | 473 | ACACATAGGGCTGTTC | 73 |
| 1495494 | 12974 | 12989 | CCATACCTCTATTTAG | 74 |
| 1495544 | 15496 | 15511 | TAGCATAAGCAGTTGT | 75 |
| 1495568 | 15994 | 16009 | CAATCATAAGTGTACC | 76 |
| 1495626 | 26288 | 26303 | AAAAGAAGTACGCTAT | 77 |
| 1495633 | 26308 | 26323 | AGAATACCACGAAAGC | 78 |
| 1495650 | 26580 | 26595 | CTATTACTAGGTTCCA | 79 |
| 1495489 | 12893 | 12908 | ACCTACAAGGTGGTTC | 80 |
| 1495506 | 13769 | 13784 | GATATATGTGGTACCA | 81 |
| 1495508 | 14404 | 14419 | CAAAACTTGTAGGTGG | 82 |
| 1495553 | 15625 | 15640 | TTCTATAGAGACACTC | 83 |
| 1495605 | 25507 | 25522 | CGAAAGGGAGTGAGGC | 84 |
| 1495647 | 26576 | 26591 | TACTAGGTTCCATTGT | 85 |
| 1495651 | 26582 | 26597 | ACCTATTACTAGGTTC | 86 |
| 1495653 | 26585 | 26600 | GAAACCTATTACTAGG | 87 |
| 1495677 | 29615 | 29630 | TACGAGAATTCATTCT | 88 |
| 1495691 | 29795 | 29810 | CACATTAGGGCTCTTC | 89 |
| 1495695 | 306 | 321 | GGCAAACTGAGTTGGA | 90 |
| 1495488 | 12892 | 12907 | CCTACAAGGTGGTTCC | 91 |
| 1495516 | 14670 | 14685 | AAAATTACCGGGTTTG | 92 |
| 1495520 | 14889 | 14904 | AATACAGCCACCATCG | 93 |
| 1495557 | 15845 | 15860 | TTAGTAAGGTCAGTCT | 94 |
| 1495559 | 15847 | 15862 | CTTTAGTAAGGTCAGT | 95 |
| 1495584 | 18890 | 18905 | CGCTTAACAAAGCACT | 96 |
| 1495593 | 20324 | 20339 | AAATCTCCATAAACGA | 97 |
| 1495610 | 26203 | 26218 | ACAAAGGCACGCTAGT | 98 |
| 1495667 | 28518 | 28533 | TAGTAGCCAATTTGGT | 99 |
| 1495678 | 29616 | 29631 | TTACGAGAATTCATTC | 100 |
| 1495680 | 29618 | 29633 | AGTTACGAGAATTCAT | 101 |
| 1495687 | 29780 | 29795 | CCATATAGGCAGCTCT | 102 |
| 1495692 | 29796 | 29811 | ACACATTAGGGCTCTT | 103 |
| 1495693 | 29800 | 29815 | TTTTACACATTAGGGC | 104 |
| 1495694 | 305 | 320 | GCAAACTGAGTTGGAC | 105 |
| 1495484 | 12845 | 12860 | TCTTAGGGAATCTAGC | 106 |
| 1495486 | 12890 | 12905 | TACAAGGTGGTTCCAG | 107 |
| 1495487 | 12891 | 12906 | CTACAAGGTGGTTCCA | 108 |
| 1495504 | 13510 | 13525 | CAGTACTAGTGCCTGT | 109 |
| 1495509 | 14405 | 14420 | CCAAAACTTGTAGGTG | 110 |
| 1495523 | 14969 | 14984 | TAAAGTCTAGCCTTAC | 111 |
| 1495542 | 15278 | 15293 | TCCCAACCCATAAGGT | 112 |
| 1495573 | 17085 | 17100 | CTTACCAGTACCAGGT | 113 |
| 1495502 | 13461 | 13476 | GCAAACCCGTTTAAAA | 114 |
| 1495507 | 14050 | 14065 | TATCTAATGTCAGTAC | 115 |
| 1495510 | 14407 | 14422 | GTCCAAAACTTGTAGG | 116 |
| 1495525 | 14973 | 14988 | ATAATAAAGTCTAGCC | 117 |
| 1495570 | 16200 | 16215 | GTACATAGCCTCATAA | 118 |
| 1495606 | 25508 | 25523 | CCGAAAGGGAGTGAGG | 119 |
| 1495643 | 26367 | 26382 | AATATTGCAGCAGTAC | 120 |
| 1495656 | 27517 | 27532 | GAAATGGTGAATTGCC | 121 |
| 1495669 | 28745 | 28760 | GAGGAAGTTGTAGCAC | 122 |
| 1495485 | 12846 | 12861 | CTCTTAGGGAATCTAG | 123 |
| 1495561 | 15886 | 15901 | TAACTAGCATTGTATG | 124 |
| 1495602 | 24620 | 24635 | CAAGATTAGCAGAAGC | 125 |
| 1495630 | 26304 | 26319 | TACCACGAAAGCAAGA | 126 |
| 1495658 | 28402 | 28417 | ATTATTGGGTAAACCT | 127 |
| 1495661 | 28406 | 28421 | CAGTATTATTGGGTAA | 128 |
| 1495689 | 29782 | 29797 | TTCCATATAGGCAGCT | 129 |
| 1495699 | 460 | 475 | GAACACATAGGGCTGT | 130 |
| 1495471 | 10412 | 10427 | CACCAGATGGTGAACC | 131 |
| 1495490 | 12894 | 12909 | AACCTACAAGGTGGTT | 132 |
| 1495533 | 15073 | 15088 | CACTAATGGCATACTT | 133 |
| 1495574 | 17096 | 17111 | GCAAAATGACTCTTAC | 134 |
| 1495590 | 19832 | 19847 | CCCAAATTATTGAGTA | 135 |
| 1495629 | 26291 | 26306 | AGAAAAAGAAGTACGC | 136 |
| 1495631 | 26306 | 26321 | AATACCACGAAAGCAA | 137 |
| 1495636 | 26334 | 26349 | AGTAAGGATGGCTAGT | 138 |
| 1495654 | 26587 | 26602 | AGGAAACCTATTACTA | 139 |
| 1495674 | 29598 | 29613 | CACAAGAGTAGACTAT | 140 |
| 1495522 | 14894 | 14909 | GCATTAATACAGCCAC | 141 |
| 1495524 | 14972 | 14987 | TAATAAAGTCTAGCCT | 142 |
| 1495567 | 15992 | 16007 | ATCATAAGTGTACCAT | 143 |
| 1495581 | 18197 | 18212 | ATCATAGAGATGAGTC | 144 |
| 1495587 | 19580 | 19595 | AGGTTATAAGTATCAA | 145 |
| 1495618 | 26276 | 26291 | CTATTAACTATTAACG | 146 |
| 1495637 | 26335 | 26350 | CAGTAAGGATGGCTAG | 147 |
| 1495521 | 14893 | 14908 | CATTAATACAGCCACC | 148 |
| 1495530 | 15045 | 15060 | AGTTATAGTAGGGATG | 149 |
| 1495540 | 15122 | 15137 | ATAGTACTACAGATAG | 150 |
| 1495541 | 15124 | 15139 | TCATAGTACTACAGAT | 151 |
| 1495547 | 15501 | 15516 | ACTATTAGCATAAGCA | 152 |
| 1495552 | 15624 | 15639 | TCTATAGAGACACTCA | 153 |
| 1495572 | 17083 | 17098 | TACCAGTACCAGGTGG | 154 |
| 1495621 | 26280 | 26295 | TACGCTATTAACTATT | 155 |
| 1495652 | 26583 | 26598 | AACCTATTACTAGGTT | 156 |
| 1495666 | 28514 | 28529 | AGCCAATTTGGTCATC | 157 |
| 1495700 | 627 | 642 | TTCTTACGAAGAAGAA | 158 |
| 1495476 | 10739 | 10754 | CCACAAGGTTAAAGTC | 159 |
| 1495481 | 12287 | 12302 | ACATTTGGGTCATAGC | 160 |
| 1495493 | 12968 | 12983 | CTCTATTTAGGTTGTT | 161 |
| 1495526 | 14974 | 14989 | CATAATAAAGTCTAGC | 162 |
| 1495609 | 25802 | 25817 | TCATAAAGTAATGGGT | 163 |
| 1495645 | 26373 | 26388 | GTTAACAATATTGCAG | 164 |
| 1495659 | 28403 | 28418 | TATTATTGGGTAAACC | 165 |
| 1495498 | 13397 | 13412 | AACTACAGCCATAACC | 166 |
| 1495537 | 15119 | 15134 | GTACTACAGATAGAGA | 167 |
| 1328721 | 15843 | 15858 | AGTAAGGTCAGTCTCA | 168 |
| 1495599 | 24449 | 24464 | AGCTAAGTTGTTTAAC | 169 |
| 1495600 | 24450 | 24465 | GAGCTAAGTTGTTTAA | 170 |
| 1495638 | 26336 | 26351 | GCAGTAAGGATGGCTA | 171 |
| 1495660 | 28404 | 28419 | GTATTATTGGGTAAAC | 172 |
| 1495686 | 29779 | 29794 | CATATAGGCAGCTCTC | 173 |
| 1495696 | 456 | 471 | ACATAGGGCTGTTCAA | 174 |
| 1495473 | 10416 | 10431 | TAAACACCAGATGGTG | 175 |
| 1495479 | 11436 | 11451 | TGATCTAAAGCATTAC | 176 |
| 1495482 | 12587 | 12602 | CACTAAGTTGAACAAT | 177 |
| 1495495 | 13272 | 13287 | CCTTTAGGATTTGGAT | 178 |
| 1495505 | 13768 | 13783 | ATATATGTGGTACCAT | 179 |
| 1495539 | 15121 | 15136 | TAGTACTACAGATAGA | 180 |
| 1495554 | 15686 | 15701 | GAGAAATGTTTACGCA | 181 |
| 1495578 | 17956 | 17971 | TATCAGACATTATGCA | 182 |
| 1495582 | 18203 | 18218 | AAACCCATCATAGAGA | 183 |
| 1495474 | 10484 | 10499 | CAACACTACCACATGA | 184 |
| 1495512 | 14516 | 14531 | GAGCTATGTAAGTTTA | 185 |
| 1495538 | 15120 | 15135 | AGTACTACAGATAGAG | 186 |
| 1495551 | 15623 | 15638 | CTATAGAGACACTCAT | 187 |
| 1495571 | 16436 | 16451 | CAATAATAGCTCATAC | 188 |
| 1495579 | 17959 | 17974 | CTCTATCAGACATTAT | 189 |
| 1495596 | 23677 | 23692 | GTTATTAGAGTAAGCA | 190 |
| 1495604 | 24663 | 24678 | CTTTTTGATTGTCCAA | 191 |
| 1495635 | 26310 | 26325 | CAAGAATACCACGAAA | 192 |
| 1495648 | 26578 | 26593 | ATTACTAGGTTCCATT | 193 |
| 1495704 | 7536 | 7551 | CCATTAACAATAGTTG | 194 |
| 1495492 | 12896 | 12911 | CAAACCTACAAGGTGG | 195 |
| 1495499 | 13398 | 13413 | CAACTACAGCCATAAC | 196 |
| 1495519 | 14854 | 14869 | CAACAACTTCAACTAC | 197 |
| 1495535 | 15110 | 15125 | ATAGAGACACCAGCTA | 198 |
| 1495549 | 15503 | 15518 | ACACTATTAGCATAAG | 199 |
| 1495562 | 15887 | 15902 | TTAACTAGCATTGTAT | 200 |
| 1495591 | 19833 | 19848 | ACCCAAATTATTGAGT | 201 |
| 1495592 | 20113 | 20128 | CTCCAATTAATGTGAC | 202 |
| 1495598 | 24448 | 24463 | GCTAAGTTGTTTAACA | 203 |
| 1495634 | 26309 | 26324 | AAGAATACCACGAAAG | 204 |
| 1495480 | 11837 | 11852 | ACATTTTAGACTGTAC | 205 |
| 1495501 | 13459 | 13474 | AAACCCGTTTAAAAAC | 206 |
| 1495511 | 14515 | 14530 | AGCTATGTAAGTTTAC | 207 |
| 1495536 | 15118 | 15133 | TACTACAGATAGAGAC | 208 |
| 1495580 | 18196 | 18211 | TCATAGAGATGAGTCT | 209 |
| 1495589 | 19781 | 19796 | GCCCAAAGCTCAAATG | 210 |
| 1495595 | 20947 | 20962 | CATTAAGATCTGAATC | 211 |
| 1495614 | 26244 | 26259 | AACGAATGAGTACATA | 212 |
| 1495655 | 27165 | 27180 | GCAAAGCAATATTGTC | 213 |
| 1495675 | 29599 | 29614 | GCACAAGAGTAGACTA | 214 |
| 1495698 | 459 | 474 | AACACATAGGGCTGTT | 215 |
| 1495496 | 13274 | 13289 | ATCCTTTAGGATTTGG | 216 |
| 1495550 | 15622 | 15637 | TATAGAGACACTCATA | 217 |
| 1495569 | 16051 | 16066 | CCTGATTAGGATGTTT | 218 |
| 1495603 | 24621 | 24636 | GCAAGATTAGCAGAAG | 219 |
| 1495644 | 26368 | 26383 | CAATATTGCAGCAGTA | 220 |
| 1495685 | 29778 | 29793 | ATATAGGCAGCTCTCC | 221 |
| 1495477 | 11432 | 11447 | CTAAAGCATTACCATA | 222 |
| 1495491 | 12895 | 12910 | AAACCTACAAGGTGGT | 223 |
| 1495532 | 15072 | 15087 | ACTAATGGCATACTTA | 224 |
| 1495545 | 15499 | 15514 | TATTAGCATAAGCAGT | 225 |
| 1495588 | 19780 | 19795 | CCCAAAGCTCAAATGC | 226 |
| 1495601 | 24474 | 24489 | ACACTTGAAATTGCAC | 227 |
| 1495657 | 27518 | 27533 | TGAAATGGTGAATTGC | 228 |
| 1495682 | 29668 | 29683 | TAAAGATTGCTATGTG | 229 |
| 1495683 | 29671 | 29686 | GATTAAAGATTGCTAT | 230 |
| 1495472 | 10414 | 10429 | AACACCAGATGGTGAA | 231 |
| 1495475 | 10491 | 10506 | TTAAAACCAACACTAC | 232 |
| 1495563 | 15888 | 15903 | TTTAACTAGCATTGTA | 233 |
| 1495583 | 18889 | 18904 | GCTTAACAAAGCACTC | 234 |
| 1495627 | 26289 | 26304 | AAAAAGAAGTACGCTA | 235 |
| 1495684 | 29689 | 29704 | TCCCTAATGTTACACA | 236 |

Modified oligonucleotides were designed as indicated in the tables below. The modified oligonucleotides are all 3-10-3 cEt gapmers (i.e., they have a central gap segment of ten 2′-deoxynucleosides flanked on each side by wing segments, each comprising three cEt modified nucleosides). The internucleoside linkages throughout each modified oligonucleotide are phosphorothioate linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are 100% complementary to the complement of genomic sequence of severe acute respiratory syndrome coronavirus 2 isolate Wuhan-Hu-1, designated herein as SEQ ID NO: 2 (the complement of GENBANK Accession No. NC_045512.2).

TABLE 2
Design of 3-10-3 cET modified oligonucleotides
complementary to the complement of severe
acute respiratory syndrome coronavirus 2
isolate Wuhan-Hu-1

| | SEQ | SEQ | | |
|---|---|---|---|---|
| | ID | ID | | |
| | NO: | NO: | | |
| | 2 | 2 | | SEQ |
| Compound | Start | Stop | Sequence | ID |
| Number | Site | Site | (5' to 3') | NO |
| 1495797 | 11627 | 11642 | ACCCGCGAAGAAGCTA | 237 |
| 1495798 | 11628 | 11643 | CACCCGCGAAGAAGCT | 238 |
| 1495810 | 1358 | 1373 | CTACCGAAGAGCTACC | 239 |
| 1495895 | 16425 | 16440 | TAAACGGGTTTGCGGT | 240 |
| 1495897 | 16427 | 16442 | TTTAAACGGGTTTGCG | 241 |
| 1495927 | 23422 | 23437 | ACTACCGAAGTTGTAG | 242 |
| 1495894 | 16424 | 16439 | AAACGGGTTTGCGGTG | 243 |
| 1495896 | 16426 | 16441 | TTAAACGGGTTTGCGG | 244 |
| 1495926 | 23421 | 23436 | CTACCGAAGTTGTAGG | 245 |
| 1495928 | 23423 | 23438 | AACTACCGAAGTTGTA | 246 |
| 1495796 | 11626 | 11641 | CCCGCGAAGAAGCTAT | 247 |
| 1495842 | 1481 | 1496 | ACCCAATAATACTGCG | 248 |
| 1495947 | 29254 | 29269 | CGTAAGAACGGTAATA | 249 |
| 1495971 | 4786 | 4801 | AAAGAAATTGACCGCC | 250 |
| 1495986 | 722 | 737 | CAAACATTGGCCGCAA | 251 |
| 1495811 | 1359 | 1374 | ACTACCGAAGAGCTAC | 252 |
| 1495901 | 16431 | 16446 | CGTTTTTAAACGGGTT | 253 |
| 1495940 | 2382 | 2397 | AACATACGAGGGCAAT | 254 |
| 1496011 | 9422 | 9437 | GCGCAAACAGGTTCAT | 255 |
| 1495792 | 1113 | 1128 | CAAAAGGCTTCTACGC | 256 |
| 1495855 | 14800 | 14815 | CAAAGAATAGAGCTCG | 257 |
| 1495938 | 2380 | 2395 | CATACGAGGGCAATTC | 258 |
| 1495967 | 4782 | 4797 | AAATTGACCGCCTCAA | 259 |
| 1495968 | 4783 | 4798 | GAAATTGACCGCCTCA | 260 |
| 1496016 | 9434 | 9449 | TTCATAACAGATGCGC | 261 |
| 1495851 | 14796 | 14811 | GAATAGAGCTCGCACC | 262 |
| 1495852 | 14797 | 14812 | AGAATAGAGCTCGCAC | 263 |
| 1495936 | 2378 | 2393 | TACGAGGGCAATTCAC | 264 |
| 1495996 | 103 | 118 | GCCTATATGGAAGAGC | 265 |
| 1496010 | 9421 | 9436 | CGCAAACAGGTTCATC | 266 |
| 1495812 | 1360 | 1375 | TACTACCGAAGAGCTA | 267 |
| 1495847 | 14708 | 14723 | GCCACTAGAGGAGCTA | 268 |
| 1495875 | 15220 | 15235 | TCAAACCCGGTAATTT | 269 |
| 1495877 | 15283 | 15298 | TAGATAAACGCACTAC | 270 |
| 1495898 | 16428 | 16443 | TTTTAAACGGGTTTGC | 271 |
| 1495913 | 17182 | 17197 | GCACTACGACAGATGT | 272 |
| 1495939 | 2381 | 2396 | ACATACGAGGGCAATT | 273 |
| 1495969 | 4784 | 4799 | AGAAATTGACCGCCTC | 274 |
| 1496015 | 9433 | 9448 | TCATAACAGATGCGCA | 275 |
| 1495814 | 1361 | 1376 | CTACTACCGAAGAGCT | 276 |
| 1495815 | 1369 | 1384 | CAAATTGGCTACTACC | 277 |
| 1495816 | 1370 | 1385 | CCAAATTGGCTACTAC | 278 |
| 1495876 | 15227 | 15242 | CAAACTGTCAAACCCG | 279 |
| 1495878 | 15284 | 15299 | CTAGATAAACGCACTA | 280 |
| 1495912 | 17181 | 17196 | CACTACGACAGATGTC | 281 |
| 1495937 | 2379 | 2394 | ATACGAGGGCAATTCA | 282 |
| 1495941 | 2383 | 2398 | GAACATACGAGGGCAA | 283 |
| 1495952 | 29634 | 29649 | CGAAAGGTAAGATGGA | 284 |
| 1495970 | 4785 | 4800 | AAGAAATTGACCGCCT | 285 |
| 1495983 | 711 | 726 | CGCAAATTGCACAATT | 286 |
| 1495984 | 712 | 727 | CCGCAAATTGCACAAT | 287 |
| 1495995 | 102 | 117 | CCTATATGGAAGAGCC | 288 |
| 1495793 | 1114 | 1129 | CCAAAAGGCTTCTACG | 289 |
| 1495846 | 14707 | 14722 | CCACTAGAGGAGCTAC | 290 |
| 1495853 | 14798 | 14813 | AAGAATAGAGCTCGCA | 291 |
| 1495889 | 16117 | 16132 | TACCACATATATCACG | 292 |
| 1495948 | 29599 | 29614 | AACGAGAAAACACACG | 293 |
| 1495845 | 14706 | 14721 | CACTAGAGGAGCTACT | 294 |
| 1495964 | 3611 | 3626 | TTAATAGTTAATAGCG | 295 |
| 1495987 | 724 | 739 | TACAAACATTGGCCGC | 296 |
| 1495994 | 101 | 116 | CTATATGGAAGAGCCC | 297 |
| 1496005 | 9111 | 9126 | AATGATGAATGTCGCA | 298 |
| 1496006 | 9112 | 9127 | TAATGATGAATGTCGC | 299 |
| 1495848 | 1491 | 1506 | CCCAAGGTTTACCCAA | 300 |
| 1495854 | 14799 | 14814 | AAAGAATAGAGCTCGC | 301 |
| 1495865 | 14879 | 14894 | CAAGATGCACTTTTCG | 302 |
| 1495883 | 15476 | 15491 | AGTTTTGGACCACTAG | 303 |
| 1495930 | 23425 | 23440 | AAAACTACCGAAGTTG | 304 |
| 1495944 | 276 | 291 | GCAGAATGAATTCTCG | 305 |
| 1495956 | 3303 | 3318 | CTAGTAATAGGTTTCC | 306 |
| 1495972 | 4787 | 4802 | AAAAGAAATTGACCGC | 307 |
| 1495862 | 14823 | 14838 | GAATCTTAAGTATGCC | 308 |
| 1495872 | 15217 | 15232 | AACCCGGTAATTTTAA | 309 |
| 1495884 | 15480 | 15495 | TACAAGTTTTGGACCA | 310 |
| 1495911 | 17045 | 17060 | GGCTAGATTCCCTAAG | 311 |
| 1495933 | 23428 | 23443 | GTGAAAACTACCGAAG | 312 |
| 1495979 | 544 | 559 | GACGCATACAAAACAT | 313 |
| 1496004 | 9092 | 9107 | TATACTCAACTGTGTC | 314 |
| 1495830 | 14038 | 14053 | ACCTTACTAAAGGACC | 315 |
| 1495831 | 14039 | 14054 | GACCTTACTAAAGGAC | 316 |
| 1495844 | 1485 | 1500 | GTTTACCCAATAATAC | 317 |
| 1495893 | 16374 | 16389 | CACTAGTACTGATGTC | 318 |
| 1495946 | 29253 | 29268 | GTAAGAACGGTAATAA | 319 |
| 1495992 | 99 | 114 | ATATGGAAGAGCCCTA | 320 |
| 1495790 | 10420 | 10435 | GCAATTTAGGTGGTGC | 321 |
| 1495822 | 13831 | 13846 | CTAATCAGGAGTATGC | 322 |
| 1495873 | 15218 | 15233 | AAACCCGGTAATTTTA | 323 |
| 1495885 | 15481 | 15496 | CTACAAGTTTTGGACC | 324 |
| 1495888 | 1610 | 1625 | TGATAATGGACCCCAA | 325 |
| 1495900 | 16430 | 16445 | _{GTTTTTAAACGGGTTT} | 326 |
| 1495929 | 23424 | 23439 | AAACTACCGAAGTTGT | 327 |
| 1495963 | 3556 | 3571 | CACTAGCCATCCTTAC | 328 |
| 1495985 | 721 | 736 | AAACATTGGCCGCAAA | 329 |
| 1496002 | 9007 | 9022 | ATAAAGGAGTTGCACC | 330 |
| 1496012 | 9430 | 9445 | TAACAGATGCGCAAAC | 331 |
| 1495802 | 1215 | 1230 | CAACTGAGGGAGCCTT | 332 |
| 1495965 | 3613 | 3628 | CGTTAATAGTTAATAG | 333 |
| 1496003 | 9008 | 9023 | GATAAAGGAGTTGCAC | 334 |
| 1496007 | 9113 | 9128 | ATAATGATGAATGTCG | 335 |
| 1496013 | 9431 | 9446 | ATAACAGATGCGCAAA | 336 |
| 1495804 | 12794 | 12809 | GGTAAGAGTCATTTTG | 337 |
| 1495821 | 13830 | 13845 | TAATCAGGAGTATGCT | 338 |
| 1495823 | 13832 | 13847 | CCTAATCAGGAGTATG | 339 |
| 1495828 | 14036 | 14051 | CTTACTAAAGGACCTC | 340 |
| 1495829 | 14037 | 14052 | CCTTACTAAAGGACCT | 341 |
| 1495867 | 14916 | 14931 | GGCTAGACTTTATTAT | 342 |
| 1495869 | 14921 | 14936 | GGTAAGGCTAGACTTT | 343 |
| 1495906 | 16916 | 16931 | CCTAAATAGAGGTATG | 344 |
| 1495920 | 18450 | 18465 | ATGCTTTAGATCAAGC | 345 |
| 1495962 | 3515 | 3530 | TGCAATATTGTTAACG | 346 |
| 1495835 | 14425 | 14440 | TTAAACCAGGTGGAAC | 347 |
| 1495882 | 15389 | 15404 | GTACATAATCAGGATG | 348 |
| 1495902 | 16499 | 16514 | GTGGAAAGGTTATGGC | 349 |
| 1495917 | 17719 | 17734 | GCTAATGGTGATTCTG | 350 |
| 1495931 | 23426 | 23441 | GAAAACTACCGAAGTT | 351 |
| 1495945 | 2724 | 2739 | GACAATATTGCTTTGC | 352 |
| 1495817 | 1371 | 1386 | ACCAAATTGGCTACTA | 353 |
| 1495824 | 13833 | 13848 | TCCTAATCAGGAGTAT | 354 |
| 1495827 | 14035 | 14050 | TTACTAAAGGACCTCA | 355 |
| 1495832 | 14056 | 14071 | AATGTTGGACTGAGAC | 356 |
| 1495864 | 14841 | 14856 | CCCTACTATAACTCAA | 357 |
| 1495977 | 542 | 557 | CGCATACAAAACATTC | 358 |
| 1495993 | 100 | 115 | TATATGGAAGAGCCCT | 359 |
| 1496014 | 9432 | 9447 | CATAACAGATGCGCAA | 360 |
| 1495794 | 1115 | 1130 | GCCAAAAGGCTTCTAC | 361 |
| 1495825 | 13834 | 13849 | ATCCTAATCAGGAGTA | 362 |
| 1495843 | 1482 | 1497 | TACCCAATAATACTGC | 363 |
| 1495874 | 15219 | 15234 | CAAACCCGGTAATTTT | 364 |
| 1495881 | 15375 | 15390 | TGTAAACTTACATAGC | 365 |
| 1495890 | 16120 | 16135 | TGGTACCACATATATC | 366 |
| 1495910 | 17037 | 17052 | TCCCTAAGAGTGATGG | 367 |
| 1495925 | 22356 | 22371 | GTACAACTATTGTTAA | 368 |
| 1495997 | 889 | 904 | GGCCAAACTGTCACTA | 369 |
| 1495789 | 10419 | 10434 | CAATTTAGGTGGTGCT | 370 |
| 1495833 | 14060 | 14075 | GCAAAATGTTGGACTG | 371 |
| 1495886 | 15482 | 15497 | CCTACAAGTTTTGGAC | 372 |
| 1495891 | 16216 | 16231 | CTAACTACCAACATGA | 373 |
| 1495915 | 193 | 208 | CATTAGGGAGGACTTG | 374 |
| 1495918 | 196 | 211 | TAACATTAGGGAGGAC | 375 |
| 1495960 | 3314 | 3329 | AACAATGGAACCTAGT | 376 |
| 1495973 | 4852 | 4867 | CATACATCACCAGATG | 377 |
| 1495988 | 725 | 740 | TTACAAACATTGGCCG | 378 |
| 1496001 | 9006 | 9021 | TAAAGGAGTTGCACCA | 379 |
| 1495791 | 10421 | 10436 | TGCAATTTAGGTGGTG | 380 |
| 1495801 | 11932 | 11947 | GCATAATGTCTGATAG | 381 |
| 1495805 | 12798 | 12813 | TACTGGTAAGAGTCAT | 382 |
| 1495813 | 13456 | 13471 | GAGGTATGAGCTATTA | 383 |
| 1495837 | 14427 | 14442 | TGTTAAACCAGGTGGA | 384 |
| 1495887 | 15484 | 15499 | CACCTACAAGTTTTGG | 385 |
| 1495942 | 24502 | 24517 | GCTAACTTTTGTGCAC | 386 |
| 1495957 | 3305 | 3320 | ACCTAGTAATAGGTTT | 387 |
| 1495991 | 97 | 112 | ATGGAAGAGCCCTAAT | 388 |
| 1496000 | 912 | 927 | CTGGTAAAGGCCAACA | 389 |
| 1495819 | 13826 | 13841 | CAGGAGTATGCTGATG | 390 |
| 1495820 | 13829 | 13844 | AATCAGGAGTATGCTG | 391 |
| 1495826 | 14003 | 14018 | CATACAATGCTAGTTA | 392 |
| 1495841 | 14434 | 14449 | CACTATATGTTAAACC | 393 |
| 1495856 | 14801 | 14816 | GCAAAGAATAGAGCTC | 394 |
| 1495892 | 16217 | 16232 | TCTAACTACCAACATG | 395 |
| 1495905 | 16915 | 16930 | CTAAATAGAGGTATGG | 396 |
| 1495908 | 16920 | 16935 | ACAACCTAAATAGAGG | 397 |
| 1495922 | 19150 | 19165 | GACTTTAACCTTGTGG | 398 |
| 1495958 | 3306 | 3321 | AACCTAGTAATAGGTT | 399 |
| 1495959 | 3307 | 3322 | GAACCTAGTAATAGGT | 400 |
| 1496009 | 942 | 957 | ACAGATTGAACCAGCT | 401 |
| 1495787 | 10417 | 10432 | ATTTAGGTGGTGCTGT | 402 |
| 1495834 | 14387 | 14402 | GCTTATGCTAATAGTG | 403 |
| 1495849 | 1492 | 1507 | CCCCAAGGTTTACCCA | 404 |
| 1495907 | 16919 | 16934 | CAACCTAAATAGAGGT | 405 |
| 1495932 | 23427 | 23442 | TGAAAACTACCGAAGT | 406 |
| 1495953 | 3300 | 3315 | GTAATAGGTTTCCTAT | 407 |
| 1495980 | 5439 | 5454 | TTAAACAACTTAGCTC | 408 |
| 1495999 | 911 | 926 | TGGTAAAGGCCAACAA | 409 |
| 1495786 | 10109 | 10124 | GCATTTGAGCTTTGGG | 410 |
| 1495836 | 14426 | 14441 | GTTAAACCAGGTGGAA | 411 |
| 1495879 | 15373 | 15388 | TAAACTTACATAGCTC | 412 |
| 1495880 | 15374 | 15389 | GTAAACTTACATAGCT | 413 |
| 1495923 | 19483 | 19498 | TACAATGGTTCACCAT | 414 |
| 1495954 | 3301 | 3316 | AGTAATAGGTTTCCTA | 415 |
| 1495955 | 3302 | 3317 | TAGTAATAGGTTTCCT | 416 |
| 1495961 | 3315 | 3330 | GAACAATGGAACCTAG | 417 |
| 1495974 | 4855 | 4870 | AATCATACATCACCAG | 418 |
| 1495800 | 11931 | 11946 | CATAATGTCTGATAGA | 419 |
| 1495809 | 13043 | 13058 | GACTATGGTGATGCTG | 420 |
| 1495838 | 14430 | 14445 | ATATGTTAAACCAGGT | 421 |
| 1495839 | 14431 | 14446 | TATATGTTAAACCAGG | 422 |
| 1495919 | 197 | 212 | GTAACATTAGGGAGGA | 423 |
| 1495978 | 543 | 558 | ACGCATACAAAACATT | 424 |
| 1495795 | 1129 | 1144 | CAAGGAACAACATTGC | 425 |
| 1495840 | 14432 | 14447 | CTATATGTTAAACCAG | 426 |
| 1495850 | 14769 | 14784 | CTCTATCTGTAGTACT | 427 |
| 1495857 | 14803 | 14818 | GTGCAAAGAATAGAGC | 428 |
| 1495860 | 14821 | 14836 | ATCTTAAGTATGCCAT | 429 |
| 1495863 | 14840 | 14855 | CCTACTATAACTCAAA | 430 |
| 1495870 | 15066 | 15081 | AACAATGTGTGATATC | 431 |
| 1495916 | 17718 | 17733 | CTAATGGTGATTCTGA | 432 |
| 1495924 | 22353 | 22368 | CAACTATTGTTAATGG | 433 |
| 1495976 | 5224 | 5239 | GGACAATCAAAAAGAG | 434 |
| 1495989 | 89 | 104 | GCCCTAATGTGTAAAA | 435 |
| 1495785 | 10057 | 10072 | TACTCAATAATTTGGG | 436 |
| 1495807 | 12928 | 12943 | CACAAGAGCACTATGT | 437 |
| 1495858 | 14818 | 14833 | TTAAGTATGCCATTAG | 438 |
| 1495859 | 14819 | 14834 | CTTAAGTATGCCATTA | 439 |
| 1495899 | 16429 | 16444 | TTTTTAAACGGGTTTG | 440 |
| 1495904 | 16914 | 16929 | TAAATAGAGGTATGGT | 441 |
| 1495909 | 16921 | 16936 | AACAACCTAAATAGAG | 442 |
| 1495935 | 23470 | 23485 | GTGGAAAATCCTACCA | 443 |
| 1495950 | 29627 | 29642 | TAAGATGGAGAGCCTT | 444 |
| 1495975 | 525 | 540 | CACCAACAGAGCCTAA | 445 |
| 1495784 | 10056 | 10071 | ACTCAATAATTTGGGT | 446 |
| 1495806 | 12867 | 12882 | TTCTAGCAATGTTGCA | 447 |
| 1495818 | 13690 | 13705 | TTTATGAGGCTATGTA | 448 |
| 1495868 | 14919 | 14934 | TAAGGCTAGACTTTAT | 449 |
| 1495981 | 5440 | 5455 | GTTAAACAACTTAGCT | 450 |
| 1496008 | 9164 | 9179 | GAAAAGTGTGACCTTC | 451 |
| 1495783 | 117 | 132 | TGCTAGGGAGAGCTGC | 452 |
| 1495799 | 11791 | 11806 | CTACACAGGCACCTAC | 453 |
| 1495803 | 12406 | 12421 | CACTAGAACCAGAATA | 454 |
| 1495808 | 12929 | 12944 | CCACAAGAGCACTATG | 455 |
| 1495943 | 24504 | 24519 | CTGCTAACTTTTGTGC | 456 |
| 1495951 | 29630 | 29645 | AGGTAAGATGGAGAGC | 457 |
| 1495990 | 783 | 798 | GTCCAGAACAAACCCA | 458 |
| 1495861 | 14822 | 14837 | AATCTTAAGTATGCCA | 459 |
| 1495871 | 15068 | 15083 | CCAACAATGTGTGATA | 460 |
| 1495903 | 183 | 198 | GACTTGAAAGAGCCAC | 461 |
| 1495921 | 18456 | 18471 | ATGGTAATGCTTTAGA | 462 |
| 1495934 | 23468 | 23483 | GGAAAATCCTACCATA | 463 |
| 1495949 | 29600 | 29615 | CAACGAGAAAACACAC | 464 |
| 1495966 | 4084 | 4099 | CATTACTTTATGATGC | 465 |
| 1495998 | 896 | 911 | ACAACAAGGCCAAACT | 466 |
| 1495788 | 10418 | 10433 | AATTTAGGTGGTGCTG | 467 |
| 1495866 | 14882 | 14897 | GATCAAGATGCACTTT | 468 |
| 1495914 | 17484 | 17499 | ACATTATCAACAATGC | 469 |
| 1495982 | 5582 | 5597 | TCTCTATGAGAACCAA | 470 |

Modified oligonucleotides were designed as indicated in the tables below. The modified oligonucleotides are all uniform MOEs (i.e., every sugar moiety in the modified oligonucleotide is a 2′-MOE modified ribosyl sugar) of 18 or 20 nucleosides in length. The internucleoside linkages throughout each modified oligonucleotide are phosphorothioate linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are 100% complementary to the genomic sequence of severe acute respiratory syndrome coronavirus 2 isolate Wuhan-Hu-1, designated herein as SEQ ID No:1 (GENBANK Accession No. NC_045512.2).

TABLE 3
Design of uniform MOE modified oligonucleotides
complementary to severe acute respiratory
syndrome coronavirus 2 isolate Wuhan-Hu-1

| | SEQ | SEQ | | |
|---|---|---|---|---|
| | ID | ID | | |
| | NO: | NO: | | |
| | 1 | 1 | | SEQ |
| Compound | Start | Stop | Sequence | ID |
| Number | Site | Site | (5' to 3') | NO |
| 1497428 | 15 | 32 | GTTTGTTACCTGGGAAGG | 471 |
| 1497429 | 16 | 33 | GGTTTGTTACCTGGGAAG | 472 |
| 1497430 | 17 | 34 | TGGTTTGTTACCTGGGAA | 473 |
| 1497431 | 18 | 35 | TTGGTTTGTTACCTGGGA | 474 |
| 1497432 | 19 | 36 | GTTGGTTTGTTACCTGGG | 475 |
| 1497433 | 20 | 37 | GGTTGGTTTGTTACCTGG | 476 |
| 1497434 | 21 | 38 | TGGTTGGTTTGTTACCTG | 477 |
| 1497435 | 22 | 39 | TTGGTTGGTTTGTTACCT | 478 |
| 1497436 | 23 | 40 | GTTGGTTGGTTTGTTACC | 479 |
| 1497437 | 24 | 41 | AGTTGGTTGGTTTGTTAC | 480 |
| 1497439 | 25 | 42 | AAGTTGGTTGGTTTGTTA | 481 |
| 1497440 | 26 | 43 | AAAGTTGGTTGGTTTGTT | 482 |
| 1497441 | 27 | 44 | GAAAGTTGGTTGGTTTGT | 483 |
| 1497442 | 28 | 45 | CGAAAGTTGGTTGGTTTG | 484 |
| 1497443 | 29 | 46 | TCGAAAGTTGGTTGGTTT | 485 |
| 1497444 | 30 | 47 | ATCGAAAGTTGGTTGGTT | 486 |
| 1497445 | 31 | 48 | GATCGAAAGTTGGTTGGT | 487 |
| 1497446 | 32 | 49 | AGATCGAAAGTTGGTTGG | 488 |
| 1497447 | 33 | 50 | GAGATCGAAAGTTGGTTG | 489 |
| 1497448 | 34 | 51 | AGAGATCGAAAGTTGGTT | 490 |
| 1497450 | 35 | 52 | AAGAGATCGAAAGTTGGT | 491 |
| 1497451 | 36 | 53 | CAAGAGATCGAAAGTTGG | 492 |
| 1497452 | 37 | 54 | ACAAGAGATCGAAAGTTG | 493 |
| 1497453 | 38 | 55 | TACAAGAGATCGAAAGTT | 494 |
| 1497454 | 39 | 56 | CTACAAGAGATCGAAAGT | 495 |
| 1497455 | 40 | 57 | TCTACAAGAGATCGAAAG | 496 |
| 1497456 | 41 | 58 | ATCTACAAGAGATCGAAA | 497 |
| 1497457 | 42 | 59 | GATCTACAAGAGATCGAA | 498 |
| 1497458 | 43 | 60 | AGATCTACAAGAGATCGA | 499 |
| 1497459 | 44 | 61 | CAGATCTACAAGAGATCG | 500 |
| 1497461 | 45 | 62 | ACAGATCTACAAGAGATC | 501 |
| 1497462 | 46 | 63 | AACAGATCTACAAGAGAT | 502 |
| 1497463 | 47 | 64 | GAACAGATCTACAAGAGA | 503 |
| 1497464 | 48 | 65 | AGAACAGATCTACAAGAG | 504 |
| 1497465 | 49 | 66 | GAGAACAGATCTACAAGA | 505 |
| 1497466 | 50 | 67 | AGAGAACAGATCTACAAG | 506 |
| 1497467 | 51 | 68 | TAGAGAACAGATCTACAA | 507 |
| 1497468 | 52 | 69 | TTAGAGAACAGATCTACA | 508 |
| 1497469 | 53 | 70 | TTTAGAGAACAGATCTAC | 509 |
| 1497492 | 54 | 71 | GTTTAGAGAACAGATCTA | 510 |
| 1497335 | 15 | 34 | TGGTTTGTTACCTGGGAAGG | 511 |
| 1497407 | 16 | 35 | TTGGTTTGTTACCTGGGAAG | 512 |
| 1497418 | 17 | 36 | GTTGGTTTGTTACCTGGGAA | 513 |
| 1497427 | 18 | 37 | GGTTGGTTTGTTACCTGGGA | 514 |
| 1497438 | 19 | 38 | TGGTTGGTTTGTTACCTGGG | 515 |
| 1497449 | 20 | 39 | TTGGTTGGTTTGTTACCTGG | 516 |
| 1497460 | 21 | 40 | GTTGGTTGGTTTGTTACCTG | 517 |
| 1497470 | 22 | 41 | AGTTGGTTGGTTTGTTACCT | 518 |
| 1497480 | 23 | 42 | AAGTTGGTTGGTTTGTTACC | 519 |
| 1497336 | 24 | 43 | AAAGTTGGTTGGTTTGTTAC | 520 |
| 1497347 | 25 | 44 | GAAAGTTGGTTGGTTTGTTA | 521 |
| 1497358 | 26 | 45 | CGAAAGTTGGTTGGTTTGTT | 522 |
| 1497366 | 27 | 46 | TCGAAAGTTGGTTGGTTTGT | 523 |
| 1497377 | 28 | 47 | ATCGAAAGTTGGTTGGTTTG | 524 |
| 1497388 | 29 | 48 | GATCGAAAGTTGGTTGGTTT | 525 |
| 1497399 | 30 | 49 | AGATCGAAAGTTGGTTGGTT | 526 |
| 1497404 | 31 | 50 | GAGATCGAAAGTTGGTTGGT | 527 |
| 1497405 | 32 | 51 | AGAGATCGAAAGTTGGTTGG | 528 |
| 1497406 | 33 | 52 | AAGAGATCGAAAGTTGGTTG | 529 |
| 1497408 | 34 | 53 | CAAGAGATCGAAAGTTGGTT | 530 |
| 1497409 | 35 | 54 | ACAAGAGATCGAAAGTTGGT | 531 |
| 1497410 | 36 | 55 | TACAAGAGATCGAAAGTTGG | 532 |
| 1497411 | 37 | 56 | CTACAAGAGATCGAAAGTTG | 533 |
| 1497412 | 38 | 57 | TCTACAAGAGATCGAAAGTT | 534 |
| 1497413 | 39 | 58 | ATCTACAAGAGATCGAAAGT | 535 |
| 1497414 | 40 | 59 | GATCTACAAGAGATCGAAAG | 536 |
| 1497415 | 41 | 60 | AGATCTACAAGAGATCGAAA | 537 |
| 1497416 | 42 | 61 | CAGATCTACAAGAGATCGAA | 538 |
| 348264 | 43 | 62 | ACAGATCTACAAGAGATCGA | 539 |
| 1497419 | 44 | 63 | AACAGATCTACAAGAGATCG | 540 |
| 348265 | 45 | 64 | GAACAGATCTACAAGAGATC | 541 |
| 1497421 | 46 | 65 | AGAACAGATCTACAAGAGAT | 542 |
| 348266 | 47 | 66 | GAGAACAGATCTACAAGAGA | 543 |
| 1497423 | 48 | 67 | AGAGAACAGATCTACAAGAG | 544 |
| 348267 | 49 | 68 | TAGAGAACAGATCTACAAGA | 545 |
| 1497425 | 50 | 69 | TTAGAGAACAGATCTACAAG | 546 |
| 348268 | 51 | 70 | TTTAGAGAACAGATCTACAA | 547 |
| 1497491 | 52 | 71 | GTTTAGAGAACAGATCTACA | 548 |

Modified oligonucleotides were designed as indicated in the tables below. The modified oligonucleotides are 16 nucleosides in length. The chemistry notation column in the tables below specifies the specific chemistry notation for modified oligonucleotides; wherein subscript ‘d’ represents a 2′-β-D-deoxyribosyl sugar moiety, subscript ‘e’ represents a 2′-MOE sugar moiety, subscript ‘k’ represents a cEt modified sugar moiety, subscript ‘s’ represents a phosphorothioate internucleoside linkage, and superscript ‘m’ before the cytosine residue (^{m}C) represents a 5-methyl cytosine. The internucleoside linkages throughout each modified oligonucleotide are phosphorothioate linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are 100% complementary to the genomic sequence of severe acute respiratory syndrome coronavirus 2 isolate Wuhan-Hu-1, designated herein as SEQ ID No:1 (GENBANK Accession No. NC_045512.2).

TABLE 4
Design of modified oligonucleotides complementary to severe
acute respiratory syndrome coronavirus 2 isolate Wuhan-Hu-1

| | SEQ | SEQ | | | SEQ |
|---|---|---|---|---|---|
| | ID | ID | | | ID |
| | NO: 1 | NO: 1 | | | NO |
| ION | Start | Stop | Sequence | | |
| Number | site | site | (5' to 3') | Chemistry Notation (5' to 3') | |
| 1497360 | 15 | 30 | TTGTTAC | TₖₛT_{dS}G_{ds}TₖₛT_{dS}A_{dS}^{m}Cₖₛ^{m}C_{ds}T_{dS}GₖₛG_{ds}G_{dS}A_{kS}A_{dS}G_{ds}Gₖ | 549 |
| | | | CTGGGAA | | |
| | | | GG | | |
| 1497361 | 16 | 31 | TTTGTTA | TₖₛT_{ds}T_{ds}GₖₛT_{ds}T_{ds}Aₖₛ^{m}C_{ds}^{m}C_{ds}TₖₛG_{ds}G_{ds}GₖₛA_{ds}A_{ds}Gₖ | 550 |
| | | | CCTGGGA | | |
| | | | AG | | |
| 1497362 | 17 | 32 | GTTTGTT | GₖₛT_{dS}T_{dS}T_{kS}G_{ds}T_{dS}T_{kS}A_{dS}^{m}C_{ds}^{m}CₖₛT_{dS}G_{ds}GₖₛG_{ds}A_{dS}Aₖ | 551 |
| | | | ACCTGGG | | |
| | | | AA | | |
| 1497363 | 18 | 33 | GGTTTGT | GₖₛG_{ds}T_{dS}T_{kS}T_{dS}G_{ds}T_{kS}T_{dS}A_{ds}^{m}Cₖₛ^{m}C_{ds}T_{ds}GₖₛG_{ds}G_{ds}Aₖ | 552 |
| | | | TACCTGG | | |
| | | | GA | | |
| 1497364 | 19 | 34 | TGGTTTG | TₖₛG_{ds}G_{ds}TₖₛT_{ds}T_{ds}GₖₛT_{ds}T_{ds}Aₖₛ^{m}C_{ds}^{m}C_{ds}TₖₛG_{ds}G_{ds}Gₖ | 553 |
| | | | TTACCTG | | |
| | | | GG | | |
| 1497365 | 20 | 35 | TTGGTTT | TₖₛT_{ds}G_{ds}GₖₛT_{ds}T_{ds}TₖₛG_{ds}T_{ds}TₖₛA_{ds}^{m}C_{ds}^{m}CₖₛT_{ds}G_{ds}Gₖ | 554 |
| | | | GTTACCT | | |
| | | | GG | | |
| 1497367 | 21 | 36 | GTTGGTT | GₖₛT_{ds}T_{ds}GₖₛG_{ds}T_{ds}TₖₛT_{ds}G_{ds}TₖₛT_{ds}A_{ds}^{m}Cₖₛ^{m}C_{ds}T_{ds}Gₖ | 555 |
| | | | TGTTACC | | |
| | | | TG | | |
| 1497368 | 22 | 37 | GGTTGGT | GₖₛG_{ds}T_{ds}TₖₛG_{ds}G_{ds}TₖₛT_{ds}T_{ds}GₖₛT_{ds}T_{ds}Aₖₛ^{m}_{cds}^{m}C_{ds}Tₖ | 556 |
| | | | TTGTTAC | | |
| | | | CT | | |
| 1497369 | 23 | 38 | TGGTTGG | TₖₛG_{ds}G_{ds}TₖₛT_{ds}G_{ds}GₖₛT_{ds}T_{ds}TₖₛG_{ds}T_{ds}TₖₛA_{ds}^{m}C_{ds}^{m}Cₖ | 557 |
| | | | TTTGTTA | | |
| | | | CC | | |
| 1497370 | 24 | 39 | TTGGTTG | TₖₛT_{dS}G_{ds}GₖₛT_{dS}T_{dS}GₖₛG_{ds}T_{dS}T_{kS}T_{dS}G_{ds}TₖₛT_{ds}A_{ds}^{m}Cₖ | 558 |
| | | | GTTTGTT | | |
| | | | AC | | |
| 1497371 | 25 | 40 | GTTGGTT | GₖₛT_{ds}T_{ds}GₖₛG_{ds}T_{ds}TₖₛG_{ds}G_{ds}TₖₛT_{ds}T_{ds}GₖₛT_{ds}T_{ds}Aₖ | 559 |
| | | | GGTTTGT | | |
| | | | TA | | |
| 1497372 | 26 | 41 | AGTTGGT | AₖₛG_{ds}T_{ds}TₖₛG_{ds}G_{ds}TₖₛT_{ds}G_{ds}GₖₛT_{ds}T_{ds}TₖₛG_{ds}T_{ds}Tₖ | 560 |
| | | | TGGTTTG | | |
| | | | TT | | |
| 1497373 | 27 | 42 | AAGTTGG | AₖₛA_{ds}G_{ds}TₖₛT_{ds}G_{ds}GₖₛT_{ds}T_{ds}GₖₛG_{ds}T_{ds}TₖₛT_{ds}G_{ds}Tₖ | 561 |
| | | | TTGGTTT | | |
| | | | GT | | |
| 1497374 | 28 | 43 | AAAGTTG | AₖₛA_{ds}A_{ds}GₖₛT_{ds}T_{ds}GₖₛG_{ds}T_{ds}TₖₛG_{ds}G_{ds}TₖₛT_{ds}T_{ds}Gₖ | 562 |
| | | | GTTGGTT | | |
| | | | TG | | |
| 1497375 | 29 | 44 | GAAAGTT | GₖₛA_{ds}A_{ds}AₖₛG_{ds}T_{ds}TₖₛG_{ds}G_{ds}TₖₛT_{ds}G_{ds}GₖₛT_{ds}T_{ds}Tₖ | 563 |
| | | | GGTTGGT | | |
| | | | TT | | |
| 1497376 | 30 | 45 | CGAAAGT | ^{m}CₖₛG_{ds}A_{ds}AₖₛA_{ds}G_{ds}TₖₛT_{ds}G_{ds}GₖₛT_{ds}T_{ds}GₖₛG_{ds}T_{ds}Tₖ | 564 |
| | | | TGGTTGG | | |
| | | | TT | | |
| 1497378 | 31 | 46 | TCGAAAG | Tₖₛ^{m}C_{ds}G_{ds}A_{kS}A_{dS}A_{dS}GₖₛT_{dS}T_{ds}GₖₛG_{ds}T_{ds}TₖₛG_{ds}G_{ds}Tₖ | 565 |
| | | | TTGGTTG | | |
| | | | GT | | |
| 1497379 | 32 | 47 | ATCGAAA | AₖₛT_{ds}^{m}C_{ds}GₖₛA_{ds}A_{ds}AₖₛG_{ds}T_{ds}TₖₛG_{ds}G_{ds}TₖₛT_{ds}G_{ds}Gₖ | 566 |
| | | | GTTGGTT | | |
| | | | GG | | |
| 1497380 | 33 | 48 | GATCGAA | GₖₛA_{ds}T_{ds}^{m}CₖₛG_{ds}A_{ds}AₖₛA_{ds}G_{ds}TₖₛT_{ds}G_{ds}GₖₛT_{ds}T_{ds}Gₖ | 567 |
| | | | AGTTGGT | | |
| | | | TG | | |
| 1497381 | 34 | 49 | AGATCGA | AₖₛG_{ds}A_{ds}TₖₛC_{ds}G_{ds}AₖₛA_{ds}A_{ds}GₖₛT_{ds}T_{ds}GₖₛG_{ds}T_{ds}Tₖ | 568 |
| | | | AAGTTGG | | |
| | | | TT | | |
| 1497382 | 35 | 50 | GAGATCG | GₖₛA_{ds}G_{ds}AₖₛT_{ds}C_{ds}GₖₛA_{ds}A_{ds}AₖₛG_{ds}T_{ds}TₖₛG_{ds}G_{ds}Tₖ | 569 |
| | | | AAAGTTG | | |
| | | | GT | | |
| 1497383 | 36 | 51 | AGAGATC | AₖₛG_{ds}A_{ds}GₖₛA_{ds}T_{ds}CₖₛG_{ds}A_{ds}AₖₛA_{ds}G_{ds}TₖₛT_{ds}G_{ds}Gₖ | 570 |
| | | | GAAAGTT | | |
| | | | GG | | |
| 1497384 | 37 | 52 | AAGAGAT | AₖₛA_{ds}G_{ds}AₖₛG_{ds}A_{ds}TₖₛC_{ds}G_{ds}AₖₛA_{ds}A_{ds}GₖₛT_{ds}T_{ds}Gₖ | 571 |
| | | | CGAAAGT | | |
| | | | TG | | |
| 1497385 | 38 | 53 | CAAGAG | CₖₛA_{ds}A_{ds}GₖₛA_{ds}G_{ds}AₖₛT_{ds}C_{ds}GₖₛA_{ds}A_{ds}AₖₛG_{ds}T_{ds}Tₖ | 572 |
| | | | ATCGAAA | | |
| | | | GTT | | |
| 1497386 | 39 | 54 | ACAAGA | AₖₛC_{ds}A_{ds}AₖₛG_{ds}A_{ds}GₖₛA_{ds}T_{ds}CₖₛG_{ds}A_{ds}AₖₛA_{ds}G_{ds}Tₖ | 573 |
| | | | GATCGAA | | |
| | | | AGT | | |
| 1497387 | 40 | 55 | TACAAGA | TₖₛA_{ds}C_{ds}AₖₛA_{ds}G_{ds}AₖₛG_{ds}A_{ds}TₖₛC_{ds}G_{ds}AₖₛA_{ds}A_{ds}Gₖ | 574 |
| | | | GATCGAA | | |
| | | | AG | | |
| 1497389 | 41 | 56 | CTACAAG | CₖₛTₐₛAₐₛCₖₛA_{ds}A_{ds}GₖₛA_{ds}G_{ds}AₖₛT_{ds}CₐₛGₛAₐₛAₐₛAₖ | 575 |
| | | | AGATCGA | | |
| | | | AA | | |
| 1497390 | 42 | 57 | TCTACAA | Tₖₛ^{m}C_{ds}T_{ds}A_{kS}^{m}C_{ds}A_{dS}AₖₛG_{ds}A_{dS}GₖₛA_{dS}T_{dS}^{m}CₖₛG_{ds}A_{dS}Aₖ | 576 |
| | | | GAGATCG | | |
| | | | AA | | |
| 1497391 | 43 | 58 | ATCTACA | AₖₛT_{ds}^{m}C_{ds}TₖₛA_{ds}^{m}C_{ds}AₖₛA_{ds}G_{ds}AₖₛG_{ds}A_{ds}Tₖₛ^{m}C_{ds}G_{ds}Aₖ | 577 |
| | | | AGAGATC | | |
| | | | GA | | |
| 1497392 | 44 | 59 | GATCTAC | GₖₛA_{ds}T_{ds}^{m}CₖₛT_{ds}A_{ds}^{m}CₖₛA_{ds}A_{ds}GₖₛA_{ds}G_{ds}AₖₛT_{ds}^{m}C_{ds}Gₖ | 578 |
| | | | AAGAGAT | | |
| | | | CG | | |
| 1497393 | 45 | 60 | AGATCTA | A_{kS}G_{ds}A_{dS}T_{kS}^{m}C_{ds}T_{ds}A_{kS}^{m}C_{ds}A_{dS}AₖₛG_{ds}A_{dS}GₖₛA_{dS}T_{dS}^{m}Cₖ | 579 |
| | | | CAAGAG | | |
| | | | ATC | | |
| 1497394 | 46 | 61 | CAGATCT | ^{m}CₖₛA_{ds}G_{ds}AₖₛT_{ds}^{m}C_{ds}TₖₛA_{ds}^{m}C_{ds}AₖₛA_{ds}G_{ds}AₖₛG_{ds}A_{ds}Tₖ | 580 |
| | | | ACAAGA | | |
| | | | GAT | | |
| 1497395 | 47 | 62 | ACAGATC | AₖₛC_{ds}A_{ds}GₖₛA_{ds}T_{ds}CₖₛTₐₛA_{ds}CₖₛAₐₛAₐₛGₖₛAₐGₐAₖ | 581 |
| | | | TACAAGA | | |
| | | | GA | | |
| 1497396 | 48 | 63 | AACAGAT | AₖₛA_{ds}CₐₛAₖGₐₛAₐₛTₖₛCₐₛT_{ds}AₖₛC_{ds}A_{ds}AₖₛG_{ds}A_{ds}Gₖ | 582 |
| | | | CTACAAG | | |
| | | | AG | | |
| 1497397 | 49 | 64 | GAACAG | GₖₛA_{ds}A_{ds}CₛAₐₛGₐₛAₖₛTₐₛC_{ds}TₖₛA_{ds}CₐₛAₖₛAₐGₐₛAₖ | 583 |
| | | | ATCTACA | | |
| | | | AGA | | |
| 1497398 | 50 | 65 | AGAACA | AₖₛG_{ds}A_{ds}AₖₛC_{ds}A_{ds}GₖₛA_{ds}T_{ds}CₖₛTₐₛA_{ds}CₖₛA_{ds}A_{ds}Gₖ | 584 |
| | | | GATCTAC | | |
| | | | AAG | | |
| 1497400 | 51 | 66 | GAGAAC | GₖₛA_{ds}G_{ds}AₖₛA_{ds}CₐₛAₖGₐₛAₐₛTₖₛCₐₛTₐₛAₖₛCₐₛAₐAₖ | 585 |
| | | | AGATCTA | | |
| | | | CAA | | |
| 1497401 | 52 | 67 | AGAGAA | AₖₛG_{ds}A_{ds}GₖₛA_{ds}A_{ds}CₖₛA_{ds}G_{ds}AₖₛT_{ds}C_{ds}TₖₛA_{ds}CₐAₖ | 586 |
| | | | CAGATCT | | |
| | | | ACA | | |
| 1497402 | 53 | 68 | TAGAGAA | TₖₛA_{ds}G_{ds}AₖₛG_{ds}A_{ds}Aₖₛ^{m}C_{ds}A_{ds}GₖₛA_{ds}T_{ds}^{m}CₖₛT_{ds}A_{ds}^{m}Cₖ | 587 |
| | | | CAGATCT | | |
| | | | AC | | |
| 1497471 | 15 | 30 | TTGTTAC | TₖₛTₑₛGₑₛTₖₛTₑₛAₑₛ^{m}Cₖₛ^{m}CₑₛTₑₛGₖₛGₑₛGₑₛAₖₛAₑₛGₑₛGₖ | 549 |
| | | | CTGGGAA | | |
| | | | GG | | |
| 1497472 | 16 | 31 | TTTGTTA | TₖₛTₑₛTₑₛGₖₛTₑₛTₑₛAₖₛ^{m}Cₑₛ^{m}CₑₛTₖₛGₑₛGₑₛGₖₛAₑₛAₑₛGₖ | 550 |
| | | | CCTGGGA | | |
| | | | AG | | |
| 1497473 | 17 | 32 | GTTTGTT | GₖₛTₑₛTₑₛTₖₛGₑₛTₑₛTₖₛAₑₛ^{m}Cₑₛ^{m}CₖₛTₑₛGₑₛGₖₛGₑₛAₑₛAₖ | 551 |
| | | | ACCTGGG | | |
| | | | AA | | |
| 1497474 | 18 | 33 | GGTTTGT | GₖₛGₑₛTₑₛTₖₛTₑₛGₑₛTₖₛTₑₛAₑₛ^{m}Cₖₛ^{m}CₑₛTₑₛGₖₛGₑₛGₑₛAₖ | 552 |
| | | | TACCTGG | | |
| | | | GA | | |
| 1497475 | 19 | 34 | TGGTTTG | TₖₛGₑₛGₑₛTₖₛTₑₛTₑₛGₖₛTₑₛTₑₛAₖₛCₑₛCₑₛTₖₛGₑₛGₑₛGₖ | 553 |
| | | | TTACCTG | | |
| | | | GG | | |
| 1497476 | 20 | 35 | TTGGTTT | TₖₛGₑₛGₑₛTₖₛTₑₛTₑₛGₖₛTₑₛTₑₛAₖₛ^{m}Cₑₛ^{m}CₑₛTₖₛGₑₛGₑₛGₖ | 554 |
| | | | GTTACCT | | |
| | | | GG | | |
| 1497477 | 21 | 36 | GTTGGTT | GₖₛTₑₛTₑₛGₖₛGₑₛTₑₛTₖₛTₑₛGₑₛTₖₛTₑₛAₑₛ^{m}Cₖₛ^{m}CₑₛTₑₛGₖ | 555 |
| | | | TGTTACC | | |
| | | | TG | | |
| 1497478 | 22 | 37 | GGTTGGT | GₖₛGₑₛTₑₛTₖₛGₑₛGₑₛTₖₛTₑₛTₑₛGₖₛTₑₛTₑₛAₖₛ^{m}Cₑₛ^{m}CₑₛTₖ | 556 |
| | | | TTGTTAC | | |
| | | | CT | | |
| 1497479 | 23 | 38 | TGGTTGG | TₖₛGₑₛGₑₛTₖₛTₑₛGₑₛGₖₛTₑₛTₑₛTₖₛGₑₛTₑₛTₖₛAₑₛ^{m}Cₑₛ^{m}Cₖ | 557 |
| | | | TTTGTTA | | |
| | | | CC | | |
| 1497481 | 24 | 39 | TTGGTTG | TₖₛTₑₛGₑₛGₖₛTₑₛTₑₛGₖₛGₑₛTₑₛTₖₛTₑₛGₑₛTₖₛTₑₛAₑₛ^{m}Cₖ | 558 |
| | | | GTTTGTT | | |
| | | | AC | | |
| 1497482 | 25 | 40 | GTTGGTT | GₖₛTₑₛTₑₛGₖₛGₑₛTₑₛTₖₛGₑₛGₑₛTₖₛTₑₛTₑₛGₖₛTₑₛTₑₛAₖ | 559 |
| | | | GGTTTGT | | |
| | | | TA | | |
| 1497483 | 26 | 41 | AGTTGGT | AₖₛGₑₛTₑₛTₖₛGₑₛGₑₛTₖₛTₑₛGₑₛGₖₛTₑₛTₑₛTₖₛGₑₛTₑₛTₖ | 560 |
| | | | TGGTTTG | | |
| | | | TT | | |
| 1497484 | 27 | 42 | AAGTTGG | AₖₛAₑₛGₑₛTₖₛTₑₛGₑₛGₖₛTₑₛTₑₛGₖₛGₑₛTₑₛTₖₛTₑₛGₑₛTₖ | 561 |
| | | | TTGGTTT | | |
| | | | GT | | |
| 1497485 | 28 | 43 | AAAGTTG | AₖₛAₑₛAₑₛGₖₛTₑₛTₑₛGₖₛGₑₛTₑₛTₖₛGₑₛGₑₛTₖₛTₑₛTₑₛGₖ | 562 |
| | | | GTTGGTT | | |
| | | | TG | | |
| 1497486 | 29 | 44 | GAAAGTT | GₖₛAₑₛAₑₛAₖₛGₑₛTₑₛTₖₛGₑₛGₑₛTₖₛTₑₛGₑₛGₖₛTₑₛTₑₛTₖ | 563 |
| | | | GGTTGGT | | |
| | | | TT | | |
| 1497487 | 30 | 45 | CGAAAGT | ^{m}CₖₛGₑₛAₑₛAₖₛAₑₛGₑₛTₖₛTₑₛGₑₛGₖₛTₑₛTₑₛGₖₛGₑₛTₑₛTₖ | 564 |
| | | | TGGTTGG | | |
| | | | TT | | |
| 1497488 | 31 | 46 | TCGAAAG | Tₖₛ^{m}CₑₛGₑₛAₖₛAₑₛAₑₛGₖₛTₑₛTₑₛGₖₛGₑₛTₑₛTₖₛGₑₛGₑₛTₖ | 565 |
| | | | TTGGTTG | | |
| | | | GT | | |
| 1497489 | 32 | 47 | ATCGAAA | AₖₛT_{eS}^{m}C_{eS}GₖₛA_{eS}A_{eS}AₖₛG_{eS}T_{eS}TₖₛG_{eS}G_{eS}TₖₛTₑₛG_{eS}Gₖ | 566 |
| | | | GTTGGTT | | |
| | | | GG | | |
| 1497490 | 33 | 48 | GATCGAA | GₖₛAₑₛTₑₛ^{m}CₖₛGₑₛAₑₛAₖₛAₑₛGₑₛTₖₛTₑₛGₑₛGₖₛTₑₛTₑₛGₖ | 567 |
| | | | AGTTGGT | | |
| | | | TG | | |
| 1497337 | 34 | 49 | AGATCGA | AₖGₑₛAₑₛTₖₛCₑₛGₑₛAₖₛAₑₛAₑₛGₖₛTₑₛTₑₛGₖₛGₑₛTₑₛTₖ | 568 |
| | | | AAGTTGG | | |
| | | | TT | | |
| 1497338 | 35 | 50 | GAGATCG | GₖₛAₑₛGₑₛAₖₛTₑₛ^{m}CₑₛGₖₛAₑₛAₑₛAₖₛGₑₛTₑₛTₖₛGₑGₑₛTₖ | 569 |
| | | | AAAGTTG | | |
| | | | GT | | |
| 1497339 | 36 | 51 | AGAGATC | AₖₛGₑₛAₑₛGₖₛAₑₛTₑₛCₖₛGₑₛAₑₛAₖₛAₑₛGₑₛTₖₛTₑₛGₑₛGₖ | 570 |
| | | | GAAAGTT | | |
| | | | GG | | |
| 1497340 | 37 | 52 | AAGAGAT | AₖₛAₑₛGₑₛAₖₛGₑₛAₑₛTₖₛCₑₛGₑₛAₖₛAₑₛAₑₛGₖₛTₑₛTₑₛGₖ | 571 |
| | | | CGAAAGT | | |
| | | | TG | | |
| 1497341 | 38 | 53 | CAAGAG | ^{m}CₖₛAₑₛAₑₛGₖₛAₑₛGₑₛAₖₛTₑₛ^{m}CₑₛGₖₛAₑₛAₑₛAₖₛGₑₛTₑₛTₖ | 572 |
| | | | ATCGAAA | | |
| | | | GTT | | |
| 1497342 | 39 | 54 | ACAAGA | Aₖₛ^{m}CₑₛAₑₛAₖₛGₑₛAₑₛGₖₛAₑₛTₑₛ^{m}CₖₛGₑₛAₑₛAₖₛAₑₛGₑₛTₖ | 573 |
| | | | GATCGAA | | |
| | | | AGT | | |
| 1497343 | 40 | 55 | TACAAGA | TₖₛAₑₛ^{m}CₑₛAₖₛAₑₛGₑₛAₖₛGₑₛAₑₛTₖₛ^{m}CₑₛGₑₛAₖₛAₑₛAₑₛGₖ | 574 |
| | | | GATCGAA | | |
| | | | AG | | |
| 1497344 | 41 | 56 | CTACAAG | ^{m}CₖₛTₑₛAₑₛ^{m}CₖₛAₑₛAₑₛGₖₛAₑₛGₑₛAₖₛTₑₛ^{m}CₑₛGₖₛAₑₛAₑₛAₖ | 575 |
| | | | AGATCGA | | |
| | | | AA | | |
| 1497345 | 42 | 57 | TCTACAA | Tₖₛ^{m}CₑₛTₑₛAₖₛ^{m}CₑₛAₑₛAₖₛGₑₛAₑₛGₖₛAₑₛTₑₛ^{m}CₖₛGₑₛAₑₛAₖ | 576 |
| | | | GAGATCG | | |
| | | | AA | | |
| 1497346 | 43 | 58 | ATCTACA | AₖₛTₑₛ^{m}CₑₛTₖₛAₑₛ^{m}CₑₛAₖₛAₑₛGₑₛAₖₛGₑₛAₑₛTₖₛ^{m}CₑₛGₑₛAₖ | 577 |
| | | | AGAGATC | | |
| | | | GA | | |
| 1497348 | 44 | 59 | GATCTAC | GₖₛAₑₛTₑₛ^{m}CₖₛTₑₛAₑₛ^{m}CₖₛAₑₛAₑₛGₖₛAₑₛGₑₛAₖₛTₑₛ^{m}CₑₛGₖ | 578 |
| | | | AAGAGAT | | |
| | | | CG | | |
| 1497349 | 45 | 60 | AGATCTA | AₖₛGₑₛAₑₛTₖₛ^{m}CₑₛTₑₛAₖₛ^{m}CₑₛAₑₛAₖₛGₑₛAₑₛGₖₛAₑₛTₑₛ^{m}Cₖ | 579 |
| | | | CAAGAG | | |
| | | | ATC | | |
| 1497350 | 46 | 61 | CAGATCT | ^{m}CₖₛAₑₛGₑₛAₖₛTₑₛ^{m}CₑₛTₖₛAₑₛ^{m}CₑₛAₖₛAₑₛGₑₛAₖₛGₑₛAₑₛTₖ | 580 |
| | | | ACAAGA | | |
| | | | GAT | | |
| 1497351 | 47 | 62 | ACAGATC | AₖₛCₑₛAₑGₖₛAₑₛTₑₛCₖₛTₑₛAₑₛCₖₛAₑₛAₑₛGₖₛAₑGₑₛAₖ | 581 |
| | | | TACAAGA | | |
| | | | GA | | |
| 1497352 | 48 | 63 | AACAGAT | AₖₛAₑₛCₑₛAₖGₑₛAₑₛTₖₛCₑₛTₑₛAₖₛCₑₛAₑₛAₓₛGₑₛAₑG | 582 |
| | | | CTACAAG | | |
| | | | AG | | |
| 1497353 | 49 | 64 | GAACAG | G.AₑₛAₑₛCₖₛAₑGₑAₖTₑₛCₑₛTₖₛAₑₛCₑₛAₖₛAₑGₑₛAₖ | 583 |
| | | | ATCTACA | | |
| | | | AGA | | |
| 1497354 | 50 | 65 | AGAACA | AₓGₑₛAₑₛAₖₛCₑₛAₑGₖₛAₑₛTₑₛCₖₛTₑₛAₑₛCₖₛAₑₛAₑₛGₖ | 584 |
| | | | GATCTAC | | |
| | | | AAG | | |
| 1497355 | 51 | 66 | GAGAAC | GₖₛAₑₛGₑₛAₖₛAₑₛCₑₛAₖGₑₛAₑₛTₖₛCₑₛTₑₛAₖₛCₑₛAₑₛAₖ | 585 |
| | | | AGATCTA | | |
| | | | CAA | | |
| 1497356 | 52 | 67 | AGAGAA | AₖGₑₛAₑₛGₖₛAₑₛAₑₛCₖₛAₑₛGₑₛAₖₛTₑₛCₑₛTₖₛAₑₛCₑₛAₓ | 586 |
| | | | CAGATCT | | |
| | | | ACA | | |
| 1497357 | 53 | 68 | TAGAGAA | TₖₛAₑₛGₑₛAₖₛGₑₛAₑₛAₖₛᵢₙCₑₛAₑₛGₖₛAₑₛTₑₛ^{m}CₖₛTₑₛAₑₛ^{m}Cₖ | 587 |
| | | | CAGATCT | | |
| | | | AC | | |
| 1497359 | 54 | 69 | TTAGAGA | TₖₛTₑₛAₑₛGₖₛAₑₛGₑₛAₖₛAₑₛCₑₛAₖₛGₑₛAₑₛTₖₛCₑₛTₑₛAₖ | 588 |
| | | | ACAGATC | | |
| | | | TA | | |
| 1497403 | 54 | 69 | TTAGAGA | TₖₛT_{ds}A_{ds}GₖₛA_{ds}G_{ds}AₖₛA_{ds}C_{ds}AₖₛG_{ds}A_{ds}TₖₛC_{ds}T_{ds}Aₖ | 588 |
| | | | ACAGATC | | |
| | | | TA | | |

### Example 2: Design of Modified Oligonucleotides Complementary to a SARS-CoV2 Nucleic Acid

Modified oligonucleotides were designed as indicated in the tables below. The modified oligonucleotides in the table below 20 nucleosides in length. The sugar motif for the modified oligonucleotides is (from 5′ to 3′): eeeeeeeeeeeeeeeeeeee; wherein each “e” represents a 2′-MOE sugar moiety. The internucleoside linkage motif for the modified oligonucleotides is (from 5′ to 3′): sssssssssssssssssss; wherein each “s” represents a phosphorothioate internucleoside linkage. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are 100% complementary to the genomic sequence of severe acute respiratory syndrome coronavirus 2 isolate Wuhan-Hu-1, designated herein as SEQ ID No:1 (GENBANK Accession No. NC_045512.2).

TABLE 5
Design of uniform MOE modified oligonucleotides
complementary to severe acute respiratory
syndrome coronavirus 2 isolate Wuhan-Hu-1

| | SEQ | SEQ | | |
|---|---|---|---|---|
| | ID NO: | ID NO: | | SEQ |
| Compound | 1 Start | 1 Stop | Sequence | ID |
| Number | Site | Site | (5' to 3') | NO |
| 1518118 | 59 | 78 | AAAGTTCGTT | 589 |
| | | | TAGAGAACAG | |
| 1518119 | 60 | 79 | TAAAGTTCGT | 590 |
| | | | TTAGAGAACA | |
| 1518120 | 61 | 80 | TTAAAGTTCG | 591 |
| | | | TTTAGAGAAC | |
| 1518121 | 62 | 81 | TTTAAAGTTC | 592 |
| | | | GTTTAGAGAA | |
| 329552 | 63 | 82 | TTTTAAAGTT | 593 |
| | | | CGTTTAGAGA | |
| 1518123 | 64 | 83 | ATTTTAAAGT | 594 |
| | | | TCGTTTAGAG | |
| 1518124 | 65 | 84 | GATTTTAAAG | 595 |
| | | | TTCGTTTAGA | |
| 1518125 | 66 | 85 | AGATTTTAAA | 596 |
| | | | GTTCGTTTAG | |
| 1518117 | 58 | 77 | AAGTTCGTTT | 597 |
| | | | AGAGAACAGA | |
| 330674 | 56 | 75 | GTTCGTTTAG | 598 |
| | | | AGAACAGATC | |
| 348271 | 57 | 76 | AGTTCGTTTA | 599 |
| | | | GAGAACAGAT | |

### Example 3: Activity of Modified Oligonucleotides Complementary to a SARS-CoV-2 RNA, In Vitro, Single Dose

Modified oligonucleotides described in the examples above were tested in vitro for activity against SARS-CoV-2.

H1437 cells were seeded at a density of 3000 cells/well in 384-well plates and treated with 10 μM of modified oligonucleotide by free uptake for 24 hours. After the 24 hour incubation, the cells were infected with SARS-CoV-2 WA1/2020 strain (BEI resources Catalog #NR-52281) at an MOI of 1 for 48 hours. Two days post infection, the cells were fixed with 4% paraformaldehyde (PFA), permeabilized with 0.03% Triton X-100, and blocked with antibody buffer (1.5% BSA, 1% goat serum, and 0.0025% Tween 20). Following blocking, cells were stained overnight with SARS-CoV-2 nucleoprotein primary antibody (ProSci Catalog #35-579, 1:2000), and then stained with anti-mouse IgG:AlexaFluor 647 secondary (Invitrogen Catalog #A21235, 1:1000), and Hoechst 33342 (Invitrogen Catalog #H3570, 1:2000). The stained cells were imaged to determine infection levels in cells treated with modified oligonucleotides.

In separate experiments, H1437 cells were seeded at a density of 3000 cells/well in 384-well plates and treated with 3 μM of modified oligonucleotide by free uptake for 24 hours. After the 24 hour incubation, the modified oligonucleotide was rinsed off the cells, and the cells were infected with SARS-CoV-2 WA1/2020 strain (BEI resources Catalog #NR-52281) at an MOI of 1 for 48 hours. Two days post infection, the cells were fixed with 4% paraformaldehyde (PFA), permeabilized with 0.03% Triton X-100, and blocked with antibody buffer (1.5% BSA, 1% goat serum, and 0.0025% Tween 20). Following blocking, cells were stained overnight with SARS-CoV-2 nucleoprotein primary antibody (ProSci Catalog #35-579, 1:2000) and then stained with anti-mouse IgG:AlexaFluor 647 secondary (Invitrogen Catalog #A21235, 1:1000), and Hoechst 33342 (Invitrogen Catalog #H3570, 1:2000). The stained cells were imaged to determine infection levels in cells treated with modified oligonucleotides.

Results are presented in the tables below as percent of the amount of infection of SARS-COV-2 in cells treated with modified oligonucleotide complementary to SARS-COV-2.

TABLE 6
Reduction of SARS-COV-2 infection by modified oligonucleotides
complementary to SARS-COV-2 in H1437 cells

% infection of SARS-COV-2

| Compound No. | no wash (10 μM) | wash (3 μM) |
|---|---|---|
| 1497335 | 0.15 | 84 |
| 1497418 | 0.12 | 93 |
| 1497427 | 0.13 | 94 |
| 1497438 | 0.85 | 89 |
| 1497449 | 2.15 | 87 |
| 1497460 | 3.69 | 103 |
| 1497470 | 1.12 | 102 |
| 1497480 | 0.14 | 90 |
| 1497336 | 0.15 | 98 |
| 1497347 | 0.23 | 97 |
| 1497358 | 0.10 | 106 |
| 1497366 | 0.25 | 93 |
| 1497377 | 0.14 | 84 |
| 1497388 | 0.13 | 93 |
| 1497399 | 2.15 | 102 |
| 1497404 | 0.29 | 100 |
| 1497405 | 0.14 | 96 |
| 1497406 | 0.21 | 95 |
| 1497408 | 1.34 | 95 |
| 1497409 | 0.54 | 94 |
| 1497410 | 1.21 | 88 |
| 1497411 | 10.88 | 100 |
| 1497412 | 32.53 | 95 |
| 1497413 | 6.08 | 102 |
| 1497414 | 23.41 | 96 |
| 1497415 | 10.88 | 98 |
| 1497416 | 15.76 | 99 |
| 348264 | 16.46 | 90 |
| 1497419 | 29.92 | 86 |
| 348265 | 13.58 | 97 |
| 1497421 | 2.64 | 95 |
| 348266 | 4.36 | 87 |
| 1497423 | 7.78 | 84 |
| 348267 | 21.20 | 92 |
| 1497425 | 29.24 | 97 |
| 348268 | 54.82 | 97 |
| 1497491 | 33.35 | 81 |
| 330674 | 6.05 | 88 |
| 348271 | 2.34 | 92 |
| 1518117 | 0.66 | 98 |
| 1518118 | 2.34 | 80 |
| 1518119 | 7.90 | 86 |
| 1518120 | 3.24 | 93 |
| 1518121 | 1.93 | 91 |
| 329552 | 4.24 | 87 |
| 1518123 | 1.57 | 82 |
| 1518124 | 0.30 | 94 |
| 1518125 | 0.54 | 94 |

TABLE 7
Reduction of SARS-COV-2 infection by modified oligonucleotides
complementary to SARS-COV-2 in H1437 cells

% infection of SARS-COV-2

| Compound No. | no wash (10 μM) | wash (3 μM) |
|---|---|---|
| 1495585 | 0.10 | 79 |
| 1495586 | 0.16 | 91 |
| 1495597 | 2.75 | 91 |
| 1495699 | 2.28 | 85 |
| 1495700 | 45.97 | 81 |
| 1495701 | 0.26 | 97 |
| 1495702 | 1.82 | 99 |
| 1495704 | 13.15 | 86 |
| 1495471 | 31.66 | 94 |
| 1495473 | 86.39 | 94 |
| 1495474 | 2.59 | 103 |
| 1495476 | 31.93 | 90 |
| 1495478 | 0.02 | 82 |
| 1495479 | 0.00 | 90 |
| 1495480 | 9.70 | 100 |
| 1495482 | 6.97 | 99 |
| 1495483 | 68.76 | 95 |
| 1495484 | 0.58 | 91 |
| 1495485 | 0.89 | 93 |
| 1495487 | 1.07 | 94 |
| 1495489 | 12.80 | 86 |
| 1495490 | 10.34 | 98 |
| 1495491 | 65.10 | 93 |
| 1495493 | 0.04 | 100 |
| 1495494 | 0.21 | 95 |
| 1495495 | 1.08 | 97 |
| 1495496 | 4.87 | 95 |
| 1495497 | 0.02 | 86 |
| 1495499 | 34.95 | 83 |
| 1495500 | 64.29 | 95 |
| 1495501 | 42.24 | 93 |
| 1495503 | 0.07 | 85 |
| 1495505 | 0.08 | 83 |
| 1495507 | 0.82 | 90 |
| 1495509 | 2.48 | 94 |
| 1495510 | 0.73 | 96 |
| 1495511 | 0.02 | 79 |
| 1495512 | 3.65 | 86 |
| 1495513 | 0.04 | 89 |
| 1495516 | 0.01 | 95 |
| 1495517 | 0.27 | 78 |
| 1495518 | 4.07 | 85 |
| 1495519 | 3.63 | 89 |
| 1495524 | 14.21 | 86 |
| 1495525 | 1.35 | 84 |
| 1495526 | 2.79 | 79 |
| 1495529 | 0.03 | 92 |
| 1495531 | 0.03 | 91 |
| 1495532 | 0.19 | 79 |
| 1495536 | 31.54 | 92 |
| 1495537 | 0.05 | 84 |
| 1495539 | 3.31 | 89 |
| 1495540 | 0.10 | 79 |
| 1495541 | 4.59 | 83 |
| 1495542 | 1.54 | 80 |
| 1495546 | 19.63 | 84 |
| 1495549 | 0.42 | 87 |
| 1495552 | 0.71 | 88 |
| 1495553 | 1.62 | 83 |
| 1495564 | 1.62 | 85 |
| 1495567 | 0.22 | 84 |
| 1495568 | 0.15 | 97 |
| 1495569 | 0.17 | 85 |
| 1495572 | 0.06 | 92 |
| 1495574 | 0.23 | 103 |
| 1495576 | 0.05 | 105 |
| 1495577 | 0.07 | 74 |
| 1495579 | 1.27 | 79 |
| 1495581 | 53.89 | 104 |
| 1495583 | 18.24 | 95 |
| 1495584 | 59.01 | 90 |
| 1495588 | 11.22 | 90 |
| 1495589 | 1.30 | 91 |
| 1495590 | 0.24 | 98 |
| 1495591 | 5.89 | 85 |
| 1495592 | 0.46 | 91 |
| 1495593 | 4.70 | 107 |
| 1495594 | 13.05 | 107 |
| 1495596 | 2.09 | 84 |
| 1495600 | 0.13 | 82 |
| 1495603 | 0.35 | 102 |
| 1495604 | 0.28 | 101 |
| 1495605 | 0.09 | 75 |
| 1495606 | 3.97 | 99 |
| 1495607 | 0.18 | 98 |
| 1495608 | 3.15 | 103 |
| 1495609 | 0.97 | 88 |
| 1495610 | 1.05 | 89 |
| 1495611 | 0.71 | 94 |
| 1495612 | 27.10 | 108 |
| 1495616 | 5.06 | 78 |
| 1495617 | 0.73 | 86 |
| 1495619 | 0.50 | 101 |
| 1495620 | 0.40 | 102 |
| 1495621 | 0.40 | 82 |
| 1495622 | 0.69 | 89 |
| 1495623 | 0.24 | 95 |
| 1495624 | 0.55 | 106 |
| 1495625 | 0.43 | 76 |
| 1495627 | 48.46 | 87 |
| 1495628 | 8.88 | 93 |
| 1495629 | 80.27 | 100 |
| 1495630 | 29.49 | 73 |
| 1495631 | 51.80 | 80 |
| 1495632 | 3.39 | 87 |
| 1495634 | 6.39 | 92 |
| 1495640 | 0.29 | 72 |
| 1495641 | 0.23 | 78 |
| 1495642 | 0.45 | 90 |
| 1495644 | 0.52 | 96 |
| 1495647 | 0.43 | 70 |
| 1495648 | 0.18 | 87 |
| 1495649 | 0.24 | 93 |
| 1495651 | 16.56 | 99 |
| 1495652 | 6.82 | 73 |
| 1495653 | 44.06 | 80 |
| 1495656 | 2.25 | 83 |
| 1495658 | 0.23 | 91 |
| 1495659 | 0.88 | 119 |
| 1495661 | 0.13 | 120 |
| 1495662 | 0.48 | 125 |
| 1495664 | 54.89 | 127 |
| 1495667 | 0.21 | 125 |
| 1495669 | 17.29 | 112 |
| 1495671 | 1.07 | 81 |
| 1495672 | 6.40 | 96 |
| 1495673 | 14.27 | 88 |
| 1495675 | 5.92 | 91 |
| 1495676 | 14.37 | 82 |
| 1495678 | 56.56 | 85 |
| 1495681 | 20.73 | 82 |
| 1495689 | 0.07 | 89 |

### Example 4: Activity of Modified Oligonucleotides Complementary to a SARS-CoV-2 RNA, In Vitro, Single Dose

Modified oligonucleotides described in the examples above were tested in vitro for activity against SARS-CoV-2.

Compound No. 792169, a control modified oligonucleotide with a sequence (from 5′ to 3′) of CGCCGATAAGGTACAC (SEQ ID NO: 600), was designed to not target SARS-CoV-2. The sugar motif for Compound No. 792169 is (from 5′ to 3′): kkkddddddddddkkk; wherein each “d” represents a 2′-β-D-deoxyribosyl sugar moiety, and each “k” represents a cEt modified sugar moiety. The internucleoside linkage motif for Compound No. 792169 is (from 5′ to 3′): sssssssssssssss; wherein each “s” represents a phosphorothioate internucleoside linkage. Each cytosine residue is a 5-methylcytosine.

H1437 cells were seeded at a density of 3000 cells/well in 384-well plates and treated with 10 μM of modified oligonucleotide by free uptake for 24 hours. After the 24 hour incubation, the modified oligonucleotide was rinsed off the cells, and the cells were infected with SARS-CoV-2 WA1/2020 strain (BEI resources Catalog #NR-52281) at an MOI of 1 for 48 hours. Two days post infection, the cells were fixed with 4% paraformaldehyde (PFA), permeabilized with 0.03% Triton X-100, and blocked with antibody buffer (1.5% BSA, 1% goat serum, and 0.0025% Tween 20). Following blocking, cells were stained overnight with SARS-CoV-2 nucleoprotein primary antibody (ProSci Catalog #35-579, 1:2000) and then stained with anti-mouse IgG:AlexaFluor 647 secondary (Invitrogen Catalog #A21235, 1:1000), and Hoechst 33342 (Invitrogen Catalog #H3570, 1:2000). The stained cells were imaged to determine infection levels in cells treated with modified oligonucleotides. Compound No. 792169 was added to the experiment as a negative control.

Results are presented in the tables below as percent of the amount of infection of SARS-COV-2 in cells treated with modified oligonucleotide that targets SARS-COV-2.

TABLE 8
Reduction of SARS-COV-2 infection by modified oligonucleotides
complementary to SARS-COV-2 in H1437 cells

| Compound Number | SARS-COV-2 % infection |
|---|---|
| 1495577 | 2 |
| 1495511 | 7 |
| 1495478 | 9 |
| 1495647 | 18 |
| 1495600 | 19 |
| 1495671 | 20 |
| 1495676 | 26 |
| 1495616 | 28 |
| 1495517 | 29 |
| 1495532 | 29 |
| 1495681 | 31 |
| 1495540 | 32 |
| 1495605 | 34 |
| 1495625 | 34 |
| 1495579 | 37 |
| 1495585 | 37 |
| 1495640 | 38 |
| 1495621 | 43 |
| 1495652 | 44 |
| 1495641 | 46 |
| 1495526 | 52 |
| 1495630 | 53 |
| 1495653 | 57 |
| 1495700 | 65 |
| 1495631 | 71 |
| 1495542 | 71 |
| 792169 | 40 |

### Example 5: Activity of Modified Oligonucleotides Complementary to a SARS-CoV-2 RNA, In Vitro, Multiple Dose

Modified oligonucleotides described in the examples above were tested in vitro for activity against SARS-CoV-2.

H1437 cells were seeded at a density of 3000 cells/well in 384-well plates and treated with modified oligonucleotide by free uptake for 24 hours at doses indicated in the table below. After the 24 hour incubation, the modified oligonucleotide was rinsed off the cells, and the cells were infected with SARS-CoV-2 WA1/2020 strain (BEI resources Catalog #NR-52281) at an MOI of 1 for 48 hours. Two days post infection, the cells were fixed with 4% paraformaldehyde (PFA), permeabilized with 0.03% Triton X-100, and blocked with antibody buffer (1.5% BSA, 1% goat serum, and 0.0025% Tween 20). Following blocking, cells were stained overnight with SARS-CoV-2 nucleoprotein primary antibody (ProSci Catalog #35-579, 1:2000) and then stained with anti-mouse IgG:AlexaFluor 647 secondary (Invitrogen Catalog #A21235, 1:1000), and Hoechst 33342 (Invitrogen Catalog #H3570, 1:2000). The stained cells were imaged to determine infection levels in cells treated with modified oligonucleotides. Compound No. 792169 (described herein above) was added to the experiment as a negative control.

Results are presented in the tables below as percent of the amount of infection of SARS-COV-2 in cells treated with modified oligonucleotide complementary to SARS-COV-2.

TABLE 9
Reduction of SARS-COV-2 infection by modified oligonucleotides
complementary to SARS-COV-2 in H1437 cells

| Compound Number | Dose (ug) | SARS-COV-2 % infection |
|---|---|---|
| 1495478 | 10 | 13 |
| | 3 | 35 |
| | 1 | 48 |
| | 0.3 | 39 |
| 1495511 | 10 | 13 |
| | 3 | 68 |
| | 1 | 76 |
| | 0.3 | 72 |
| 1495577 | 10 | 7 |
| | 3 | 57 |
| | 1 | 74 |
| | 0.3 | 95 |
| 1497335 | 10 | 25 |
| | 3 | 60 |
| | 1 | 78 |
| | 0.3 | 104 |
| 1497377 | 10 | 34 |
| | 3 | 70 |
| | 1 | 91 |
| | 0.3 | 113 |
| 792169 | 10 | 59 |
| | 3 | 79 |
| | 1 | 96 |
| | 0.3 | 110 |

What is claimed:

1. A compound comprising a modified oligonucleotide consisting of 8 to 80 linked nucleosides and having a nucleobase sequence comprising at least 8 contiguous nucleobases of any of the nucleobase sequences of SEQ ID NOs: 3-599.

2. A compound comprising a modified oligonucleotide consisting of 9 to 80 linked nucleosides and having a nucleobase sequence comprising at least 9 contiguous nucleobases of any of the nucleobase sequences of SEQ ID NOs: 3-599.

3. A compound comprising a modified oligonucleotide consisting of 10 to 80 linked nucleosides and having a nucleobase sequence comprising at least 10 contiguous nucleobases of any of the nucleobase sequences of SEQ ID NOs: 3-599.

4. A compound comprising a modified oligonucleotide consisting of 11 to 80 linked nucleosides and having a nucleobase sequence comprising at least 11 contiguous nucleobases of any of the nucleobase sequences of SEQ ID NOs: 3-599.

5. A compound comprising a modified oligonucleotide consisting of 12 to 80 linked nucleosides and having a nucleobase sequence comprising at least 12 contiguous nucleobases of any of the nucleobase sequences of SEQ ID NOs: 3-599.

6. A compound comprising a modified oligonucleotide consisting of 16 to 80 linked nucleosides and having a nucleobase sequence comprising the nucleobase sequence of any one of SEQ ID NOs: 3-470 or 549-588.

7. A compound comprising a modified oligonucleotide consisting of 18 to 80 linked nucleosides and having a nucleobase sequence comprising the nucleobase sequence of any one of SEQ ID NOs: 471-510.

8. A compound comprising a modified oligonucleotide consisting of 20 to 80 linked nucleosides and having a nucleobase sequence comprising the nucleobase sequence of any one of SEQ ID NOs: 511-548 or 589-599.

9. A compound comprising a modified oligonucleotide having a nucleobase sequence consisting of the nucleobase sequence of any one of SEQ ID NOs: 3-599.

10. The compound of any one of claims 1-9, wherein at least one internucleoside linkage of the modified oligonucleotide is a modified internucleoside linkage, at least one nucleoside of the modified oligonucleotide comprises a modified sugar, or at least one nucleobase of the modified oligonucleotide is a modified nucleobase.

11. The compound of claim 10, wherein the modified internucleoside linkage is a phosphorothioate internucleoside linkage.

12. The compound of claim 10 or 11, wherein the modified sugar is a bicyclic sugar.

13. The compound of claim 12, wherein the bicyclic sugar is selected from the group consisting of: 4′-(CH₂)—O-2′ (LNA); 4′-(CH₂)₂—O-2′ (ENA); and 4′-CH(CH₃)—O-2′ (cEt).

14. The compound of claim 10 or 11, wherein the modified sugar is 2′-O-methoxyethyl.

15. The compound of any one of claims 10-14, wherein the modified nucleobase is a 5-methylcytosine.

16. The compound of any one of claims 1-15, wherein the modified oligonucleotide has:
a gap segment consisting of linked 2′-deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

17. A modified oligonucleotide consisting of 16 to 80 linked nucleobases and having a nucleobase sequence comprising the nucleobase sequence of any one of SEQ ID NOs: 3-470, wherein the modified oligonucleotide has:
a gap segment consisting of ten linked 2′-deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine.

18. A modified oligonucleotide consisting of 18 to 80 linked nucleobases and having a nucleobase sequence comprising the nucleobase sequence of any one of SEQ ID NOs: 471-510, wherein each nucleoside of the modified oligonucleotide comprises a 2′-MOE nucleoside; wherein each internucleoside linkage is a phosphorothioate linkage; and wherein each cytosine is a 5-methylcytosine.

19. A modified oligonucleotide consisting of 20 to 80 linked nucleobases and having a nucleobase sequence comprising the nucleobase sequence of any one of SEQ ID NOs: 511-548 or 589-599, wherein each nucleoside of the modified oligonucleotide comprises a 2′-MOE nucleoside; wherein each internucleoside linkage is a phosphorothioate linkage; and wherein each cytosine is a 5-methylcytosine.

20. A modified oligonucleotide consisting of 16 linked nucleobases and having a nucleobase sequence consisting of the nucleobase sequence of any one of SEQ ID NOs: 549-588, wherein the modified oligonucleotide comprises the sugar motif: kddkddkddkddkddk in the 5′ to 3′ direction, wherein “k” indicates a cEt sugar moiety and “d” indicates an unmodified 2′-deoxyribosyl sugar moiety; wherein each internucleoside linkage is a phosphorothioate linkage; and wherein each cytosine is a 5-methylcytosine.

21. A modified oligonucleotide consisting of 16 linked nucleobases and having a nucleobase sequence consisting of the nucleobase sequence of any one of SEQ ID NOs: 549-588, wherein the modified oligonucleotide comprises the sugar motif: keekeekeekeekeek in the 5′ to 3′ direction, wherein “k” indicates a cEt sugar moiety and “e” indicates 2′-MOE sugar moiety; wherein each internucleoside linkage is a phosphorothioate linkage; and wherein each cytosine is a 5-methylcytosine.

22. The compound of any one of claims 1-21, wherein the oligonucleotide is at least 80%, 85%, 90%, 95% or 100% complementary to SEQ ID NO:1 or 2.

23. The compound of any one of claims 1-22, wherein the compound is single-stranded.

24. The compound of any one of claims 1-22, wherein the compound is double-stranded.

25. The compound of any one of claims 1-22, wherein the compound comprises ribonucleotides.

26. The compound of any one of claims 1-22, wherein the compound comprises deoxyribonucleotides.

27. The compound of any one of claims 1-21, wherein the modified oligonucleotide consists of 16 to 30 linked nucleosides or 18 to 30 linked nucleosides, or 20 to 30 linked nucleosides.

28. The compound of any one of claims 1-27, wherein the compound consists of the modified oligonucleotide.

29. A compound consisting of a pharmaceutically acceptable salt of any of the compounds of claims 1-28.

30. The compound of claim 29, wherein the pharmaceutically acceptable salt is a sodium salt.

31. The compound of claim 30, wherein the pharmaceutically acceptable salt is a potassium salt.

32. A composition comprising the compound of any one of claims 1-31 and a pharmaceutically acceptable diluent or carrier.

33. A composition comprising the compound of any one of claims 1-31 and water.

34. A composition comprising a compound of any one of claims 1-32, for use in therapy.

35. A method of inhibiting or reducing SARS-CoV-2 replication, infectivity, viral titer, or viral load in lung cells comprising contacting the lung cells with the compound of any one of claims 1-31 or composition of any one of claims 32-34, thereby inhibiting or reducing SARS-CoV-2 replication, infectivity, viral titer, or viral load in the lung cells.

36. A method of inhibiting or reducing SARS-CoV-2 replication, infectivity, viral titer, or viral load in an individual comprising administering to the individual the compound of any one of claims 1-31 or composition of any one of claims 32-34, thereby inhibiting or reducing SARS-CoV-2 replication, infectivity, viral titer, or viral load in the individual.

37. A method of preventing or treating COVID-19 in an individual comprising administering to the individual the compound of any one of claims 1-31 or composition of any one of claims 32-34, thereby preventing or treating COVID-19 in the individual.

38. The method of any of claims 35-37, wherein contacting or administering the compound of any one of claims 1-31 or composition of any one of claims 32-34 prevents or improves a COVID-19 symptom.

39. The method of claim 38, wherein the COVID symptom is respiratory illness, difficulty breathing, fever, cough, fatigue, aches and pains, sore throat, runny nose, diarrhea, loss of taste or smell, or nasal congestion.

40. Use of the compound of any one of claims 1-31 or composition of any one of claims 32-34 for inhibiting or reducing SARS-CoV-2 replication, infectivity, viral titer, or viral load in lung cells.

41. Use of the compound of any one of claims 1-31 or composition of any one of claims 32-34 for inhibiting or reducing SARS-CoV-2 replication, infectivity, viral titer, or viral load in an individual.

42. Use of the compound of any one of claims 1-31 or composition of any one of claims 32-34 for preventing or treating COVID-19 in an individual.

43. The use of any of claims 40-42, for preventing or improving a COVID-19 symptom.

44. The use of claim 43, wherein the COVID symptom is respiratory illness, difficulty breathing, fever, cough, fatigue, aches and pains, sore throat, runny nose, diarrhea, loss of taste or smell, or nasal congestion.

45. Use of the compound of any one of claims 1-31 or composition of any one of claims 32-34 for the preparation or manufacture of a medicament for inhibiting or reducing SARS-CoV-2 replication, infectivity, viral titer, or viral load in lung cells.

46. Use of the compound of any one of claims 1-31 or composition of any one of claims 32-34 for the preparation or manufacture of a medicament for inhibiting or reducing SARS-CoV-2 replication, infectivity, viral titer, or viral load in an individual.

47. Use of the compound of any one of claims 1-31 or composition of any one of claims 32-34 for the preparation or manufacture of a medicament for preventing or treating COVID-19 in an individual.

48. The use of any of claims 40-42, for the preparation or manufacture of a medicament for preventing or improving a COVID-19 symptom.

49. The use of claim 43, wherein the COVID symptom is respiratory illness, difficulty breathing, fever, cough, fatigue, aches and pains, sore throat, runny nose, diarrhea, loss of taste or smell, or nasal congestion.


SEQUENCE LISTING (600 sequences)

SEQ ID NO: 1 - DNA, 29903 bp, Homo sapiens
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29903 bp, Homo sapiens
tttttttttttttttttttttttttttttttttgtcattctcctaagaagctattaaaatcacatggggatagcactactaaaattaattttacacattagggctcttccatataggcagctctccctagcattgttcactgtacactcgatcgtactccgcgtggcctcggtgaaaatgtggtggctctttcaagtcctccctaatgttacacactgattaaagattgctatgtgagattaaagttaactacatctacttgtgctatgtagttacgagaattcattctgcacaagagtagactatatatcgtaaacggaaaagcgaaaacgtttatatagcccatctgccttgtgtggtctgcatgagtttaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttctttttgtcctttttaggctctgttggtgggaatgttttgtatgcgtcaatatgcttattcagcaaaatgacttgatctttgaaatttggatctttgtcatccaatttgatggcacctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttggggccgacgttgttttgatcgcgccccactgcgttctccattctggttactgccagttgaatctgagggtccaccaaacgtaatgcggggtgcatttcgctgattttggggtccattatcagacattttagtttgttcgtttagatgaaatctaaaacaacacgaacgtcatgatactctaaaaagtcttcatagaacgaacaacgcactacaagactacccaatttaggttcctggcaattaattgtaaaaggtaaacaggaaactgtataattaccgatatcgatgtactgaatgggtgatttagaaccagcctcatccacgcacaattcaattaaaggtgctgattttctagctcctactctaatataccatttagaatagaagtgaataggacacgggtcatcaactacatatggttgatgttgagtacatgactgtaaactacattcttggtgaaatgcagctacagttgtgatgattcctaagaaaacaagaaatttcatgttcgtttaggcgtgacaagtttcattatgatcttgcagttcaagtgagaaccaaaagataataagcataattaaaacaaggaatagcagaaaggctaaaaagcacaaatagaagtcaattaatgaaagttcaatcattctgtctttcttttgagtgtgaagcaaagtgttataaacactattgccgcaacaataagaaaaattggagagtaaagttcttgaacttcctcttgtctgatgaacagtttaggtgaaactgatctggcacgtaactgatagacgtgttttacgccgtcaggacaagcaaaagcaaattgagtgctaaagcaagtcagtgcaaatttgttatcagctagaggatgaaatggtgaattgccctcgtatgttccagaagagcaaggttcttttaaaagtactgttgtacctctaacacactcttggtagtgataaagctcacaagtagcgagtgttatcagtgccaagaaaagaataattttcatgttcgtttaatcaatctccattggttgctcttcatctaattgagaatatttattctcagttagtgacttagataaatttttaattatgaggtttatgatgtaatcaagattccaaatggaaactttaaaagtcctcataataattagtaatatctctgctatagtaacctgaaagtcaacgagatgaaacatctgttgtcacttactgtacaagcaaagcaatattgtcactgctactggaatggtctgtgtttaatttatagttgccaatcctgtagcgactgtatgcagcaaaacctgagtcacctgctacacgctgcgaagctcccaatttgtaataagaaagcgttcgtgatgtagcaacagtgatttctttaggcaggtccttgatgtcacagcgtcctagatggtgtccagcaatacgaagatgtccacgaaggatcacagctccgattacgagttcactttctagaagcggtctggtcagaatagtgccatggagtggcacgttgagaagaatgttagtttctggattgaatgaccacatggaacgcgtacgcgcaaacagtctgaaagaagcaatgaagtagctgagccacatcaagcctacaagacaagccattgcgatagcaattccaccggtgatccaatttattctgtaaacagcagcaagcacaaaacaagctaaagttactggccataacagccagaggaaaattaacttaattatatacaaaaacctattcctgttggcataggcaaattgtagaagacaaatccatgtaaggaataggaaacctattactaggttccattgttcaaggagctttttaagctcttcaacggtaatagtaccgttggaatctgccatggctaaaattaaagttccaaacagaaaaactaatataatatttagttcgtttagaccagaagatcaggaactctagaagaattcagatttttaacacgagagtaaacgtaaaaagaaggttttacaagactcacgttaacaatattgcagcagtacgcacacaatcgaagcgcagtaaggatggctagtgtaactagcaagaataccacgaaagcaagaaaaagaagtacgctattaactattaacgtacctgtctcttccgaaacgaatgagtacataagttcgtactcatcagcttgtgcttacaaaggcacgctagtagtcgtcgtcggttcatcataaattggttccattactggattaacaactccggatgaaccgtcgattgtgtgaatttggacatgttcttcaggctcatcaacaattttattgtagatgaagaaggtaacatgttcaacaccagtgtctgtactcaattgagttgagtacagctggtaatagtctgaagtgaagtaactgtgtaatacaacacagtcttttactccagattcccatttttcagtataaccaccaatctggtagtcatgttcagaaataggacttgttgtgccatcacctgaagtaatgacaattgaagaagttacactattgtaaggtatacaatagtcgtaacaattagtatgccagcaaagaaaatagttggcatcataaagtaatgggtttttggaacggcatttccagcaaagccaaagcctcattattattcttacaaagtttatactctgcaagaagtagactaaagcataaagatagagaaaaggggcttcaaggccagcagcaacgagcaaaaggtgtgagtaaactgttacaaacaacaacagcaagttgcaaacaaagtgaacacccttggagagtgctagttgccatctctttttgagggttatgattttggaagcgctctgaaaaacagcaagaagtgcaacgccaacaataagccatccgaaagggagtgaggcttgtatcggtatcgttgcagtagcgcgaacaaaatctgaaggagtagcatccttgatttcaccttgcttcaaagttacagttccaattgtgaagattctcataaacaaatccataagttcgtttatgtgtaatgtaatttgactcctttgagcactggctcagagtcgtcttcatcaaatttgcagcaggatccacaagaacaacagcccttgagacaactacagcaactggtcatacagcaaagcataattgtcaccattactatggcaatcaagccagctataaaacctagccaaatgtaccatggccattttatatactgctcatactttccaagttcttggagatcgatgagagattcatttaaattcttggcaacctcattgaggcggtcaatttctttttgaatgtttacaactgaagcattaatgccagagatgtcacctaaatcaacatctggtgatgtatgattcttaaaatatttatctaactcctccttgaatgagtctaattcaggttgcaaaggatcataaactgtgttgttgacaattcctattacaacatcacagttaccagacacaaatgtgttgtctgtagtaatgatttgtggttcataaaaattcctttgtgttacaaaccagtgtgtgccatttgaaacaaagacaccttcacgaggaaagtgtgcttttccatcatgacaaatggcaggagcagttgtgaagttcttttcttgtgcagggacataagtcacatgcaagaagactacaccatgaggtgctgactgagggaaggacataagatgatagccctttccacaaaaatcaactctttttgattgtccaagtacacactctgacattttagtagcagcaagattagcagaagctctgatttctgcagctctaattaattgttgagtcacatatgtctgcaaactttgaagtctgcctgtgatcaacctatcaatttgcacttcagcctcaactttgtcaagacgtgaaaggatatcatttaaaacacttgaaattgcaccaaaattggagctaagttgtttaacaagcgtgtttaaagcttgtgcattttggttgaccacatcttgaagttttccaagtgcacttgctgtggaagaaagtgagtcttgaattttgccaatagcactattaaattggttggcaatcaatttttggttctcatagagaacattctgtgtaactccaataccattaaacctataagccatttgcatagcaaatggtatttgtaatgcagcacctgcaccaaaggtccaaccagaagtgattgtacccgctaacagtgcagaagtgtattgagcaatcatttcatctgtgagcaaaggtggcaaaacagtaaggccgttaaacttttgtgcacaaatgaggtctctagcagcaatatcaccaaggcaatcaccatattgtttgatgaagccagcatctgcaagtgtcactttgttgaaaagtagatcttcaataaatgacctcttgcttggttttgatggatctggtaatatttgtgaaaaattaaaaccaccaaaatctttaattggtggtgttttgtaaatttgtttgacttgtgcaaaaacttcttgggtgtttttgtcttgttcaacagctattccagttaaagcacggtttaattgtgtacaaaaactgccatattgcaacaaaagattgctgcattcagttgaatcaccacaaatgtacattgtacaatctactgatgtcttggtcatagacactggtagaatttctgtggtaacactaatagtaaaatttgtgggtatggcaatagagttattagagtaagcaactgaattttctgcaccaagtgacatagtgtaggcaatgatggattgactagctacactacgtgcccgccgaggagaattagtctgagtctgataactagcgcatatacctgcaccaatgggtatgtcacactcatatgagttgttgacatgttcagcccctattaaacagcctgcacgtgtttgaaaaacattagaacctgtagaataaacacgccaagtaggagtaagttgatctgcatgaatagcaacagggacttctgtgcagttaacatcctgataaagaacagcaacctggttagaagtatttgttcctggtgttataacactgacaccaccaaaagaacatggtgtaatgtcaagaatctcaagtgtctgtggatcacggacagcatcagtagtgtcagcaatgtctctgccaaattgttggaaaggcagaaactttttgttagactcagtaagaacacctgtgcctgttaaaccattgaagttgaaattgacacatttgtttttaaccaaattagtagactttttaggtccacaaacagttgctggtgcatgtagaagttcaaaagaaagtactactactctgtatggttggtaaccaacaccattagtgggttggaaaccatatgattgtaaaggaaagtaacaattaaaaccttcaacaccattacaaggtgtgctaccggcctgatagatttcagttgaaatatctctctcaaaaggtttgagattagacttcctaaacaatctatacaggtaattataattaccaccaaccttagaatcaagattgttagaattccaagctataacgcagcctgtaaaatcatctggtaatttataattataatcagcaatctttccagtttgccctggagcgatttgtctgacttcatcacctctaattacaaatgaatctgcatagacattagtaaagcagagatcatttaatttagtaggagacactccataacacttaaaagtggaaaatgatgcggaattatataggacagaataatcagcaacacagttgctgattctcttcctgttccaagcataaacagatgcaaatctggtggcgttaaaaacttcaccaaaagggcacaagtttgtaatattaggaaatctaacaatagattctgttggttggactctaaagttagaagtttgatagattcctttttctacagtgaaggatttcaacgtacactttgtttctgagagagggtcaagtgcacagtctacagcatctgtaatggttccattttcattatattttaatagaaaagtcctaggttgaagataacccacataataagctgcagcaccagctgtccaacctgaagaagaatcaccaggagtcaaataacttctatgtaaagcaagtaaagtttgaaacctagtgatgttaatacctattggcaaatctaccaatggttctaaagccgaaaaaccctgagggagatcacgcactaaattaataggcgtgtgcttagaatatattttaaaataaccatcaatattcttaaacacaaattccctaagatttttgaaattaccctgttttccttcaaggtccataagaaaaggctgagagacatattcaaaagtgcaattattcgcactagaataaactctgaactcactttccatccaacttttgttgtttttgtggtaataaacacccaaaaatggatcattacaaaattgaaattcacagactttaataacaacattagtagcgttattaacaataagtagggactgggtcttcgaatctaaagtagtaccaaaaatccagcctcttattatgttagacttctcagtggaagcaaaataaacaccatcattaaatggtaggacagggttatcaaacctcttagtaccattggtcccagagacatgtatagcatggaaccaagtaacattggaaaagaaaggtaagaacaagtcctgagttgaatgtaaaactgaggatctgaaaactttgtcagggtaataaacaccacgtgtgaaagaattagtgtatgcagggggtaattgagttctggttgtaagattaacacactgactagagactagtggcaataaaacaagaaaaacaaacattgttcgtttagttgttaacaagaacatcactagaaataacaactctgttgttttctctaattataagtctacctttactaagaagagataaaatcatatcattgatttgaccttcttttaaagacataacagcagtaccccttaatttaaggggaaatttactcatgtcaaataaagaataggaagacaactgaattggatttgtattcctccaaaatatgtaatttgcatgcatgacataaccatctatttgttcgcgtggtttgccaagataattacatccaattaaaaatgcttcagatgatgacgcattcacattagtaacaaaggctgtccaccatgcgaagtgtcccatgagcttataaagatcagcattccaagaatgttctgttatctttatagccacggaacctccaagagctagcttttgttgtataaacccacaaatgtaagtgaaaaaaccctctttagagtcattttcttttgtaacatttttagtcttagggtcgtacatatcactaataatgagatcccatttattagctgtatgtacagttgcacaatcaccaatcaaagttgaatctgcatcagagacaaagtcattaagatctgaatcgacaagcagcgtacccgtaggcaaccactgtcttaaaacagctgtacctggtgcaactcctttatcagaaccagcaccaaaatgtataactctcatattatagggtacagctaatgttaatgtgtttaaatattgacacagttgagtatattttgcgacattcatcattatgcctttaggtaatgttgcactatcaccataattttgaaggtcacacttttctaatagcattctttgcattttgtaaagattaggcatagcaacacccggttgccacgcttgactagattgtaattttgggtaaaatgtttctacatggccatctttacaccaaagcataaatgaaatttctgtatagtcaatagtcactttgacaaccttagaaactacagataaatcttgggattttattatttcaacaaaatcatcaagtaataaatcaataacagaacacacacacttagatgaacctgtttgcgcatctgttatgaaatagtttttaactgtactgtccataggaataaaatcttctaattcaaaaggtgattccttaaaacgtttagctagtccaatcagtagatgtaaaccacctaactgactatgactaaaatctccataaacgatatgttcgaaggcatagccttctaatttataccgttcaatgaattcatccatagctaattctaagaaatcaatttccatttgactcctgggtttaaattcttgtaaatttctactctgagtaaagtaagtttcaggtaattgttggacaacaccatcaactttcttataataattgaactgtgtttttacggcttctccaattaatgtgactccattaagactagcttgtttgggacctacagatggttgtaaacctttaacactaccttctgtaataagaacaccattacgggcatttctaaataagtctacttgaccatcaactctaccatcaaaaaagacagtgagtggtgcacaaatcgtttcagttggtttcttggctatgtcagtcatagaacaaacaccaatagtagatatatgtgctggagcatctcttttgtagtcccagatcacagtattagcagcaatgtccacacccaaattattgagtattttcacctctggtactggtttaatgttgcgcttagcccaaagctcaaatgctacattaacaggtaatgttgttttattttcaaacaattctacatcaacaccatcaacttttgtgtaaacagtgttattaatgatagaaactggtacttcaccctgttgtccatcaaagtgtcccttatttacaacattaaaagccacattttctaaactctgaagtcttgtaaaagtgttccagaggttataagtatcaaattgtttgtaaacccacaagctaaagccagctgagatcatcatgttataagcatcgagatacaatctgtactcattagcatgatgtctacagacagcaccacctaaattgcaacgtgttatacacgtagcagactttagtggtacataatctatatctgacactacttgttttccatgagactcacatggactgtcagagtaatagaaaaatggtaattgttttaaattaacaaaagcacttttatcaaaagctggtgtgtggaatgcatgtttatttacatacaaactgccaccatcacaaccaggcaagttaaggttagatagcactctagtgtcaaatctacaaacaatggaattagcaggatatctatcgacattgcaattccaaaataggcatacaccatctgtgaatttgtcagaatgtgtggcataagaatagaataattcttctattttataagctttgtcactacaaggctgtgcatcatagaacttccattctacatcagcttgaggtacacacttaatagctttagggttaccaatgtcgtgaagaactgggaatttgtctgctaataatgcagctttaacaaccatgtgttgaacctttctacaagccgcattaatcttcagttcatcaccaattataggatattcaatagtccagtcaacacgcttaacaaagcactcgtggacagctagacacctagtcatgattgcatcacaactagctacatgtgcattaccatggacttgacaatacagatcatggttgctttgtaggttacctgtaaaaccccattgttgaacatcaatcataaacggattatagacgtaatcaaatccaatagaatgatgccaacaggcataagtgtctgaagcagtggaaaagcatgtggcacgtctatcacatagacaacaggtgcgctcaggtcctattttcacaaaatacttcatagatgtcaactcaaagccatgtgcccataagacaaatacgactctgtcagagagatttttaagtgtgtcacttaacatttgtacaatctttatacgcactacattccaaggaagtcctttgtacataagtggtatgaggtgtttaaattgatctccaggcggtggtttagcactaactctggaaaaatctgtattattaggtgtatcaacataacctgtaggtacagcaactaggttaacacctgtagaaaaacctagctgtaaaggtaaattggtaccaacagcttctctagtagcatgacacccctcgacatcgaagccaatccatgcacgtacatgtcttatagcttcttcgcgggtgataaacatgttagggtaaccattaacttgataattcattttaaaacccatcatagagatgagtcttctataggtcatgtccttaggtatgccaggtatgtcaacacataaaccttcagttttgaatttagtgtcaacactgaggtgtgtaggtgcctgtgtaggatgtaacccagtgattaccttactacaatctttaaagagtcctgttacattttcagcttgtaaagttgccacattcctacgtggaatttcaagacttgtaaattgcaacttgtcataaaggtctctatcagacattatgcaaagtatgcctacttttgctctggtaatagcaacattaaatctgtttacattacaagagtgagctgtttcagtggtttgagtgaatatgacatagtcatattctgagccctgtgatgaatcaacagtttgagttggtagtcccaaaatctttgaggctacagcattctgtgaattataaggtgaaataaagacagcttttctccaagcagggttacgtgtaaggaattctcttaccacgcctatttgtggcctgttaattgcagatgaaacatcatgcgtgataacacccttataaaacattttaaagcattgagctgatttgtctttatgtgctttaagcttattatcataaaccaaagcactcacagtgtcaacaatttcagcaggacaacgccgacaagttccgaggaacatgtctggacctatagttttcataagtctacacactgaattgaaatattctggttctagtgtgcccttagttagcaatgtgcgtggtgcaggtaattgagcagggtcgccaatgtacacatagtgcttagcacgtaatctggcattgacaacactcaaatcataatttgtggccattgaaatttcatcaaagacaactatatctgctgtcgtctcaggcaatgcatttacagtacaaaagacatactgttctaatgttgaattcactttgaatttatcaaaacactctacacgagcacgtgcaggtataattctactacatttatctataggcaaatattttaatgccttctcacatagtgcatcaacagcggcatgagagcaagctgtatacactatgcgagcagaagggtagtagagagctaggccaatagcaaaatgactcttaccagtaccaggtggtccctggagtgtagaatacttttgcataccaaccttttgataatttgcaacattgctagaaaactcatctgagatattgagtgttgggtataagccagtaattctaacatagtgctcttgtggcactagtgtaggtgcacttaatggcattactgtatgtgatgtcagcacaaaataatcaccaacatttaatttgtaagttgttgtacctcggtaaacaacagcatcaccatagtcacctttttcaaaggtgtactctcctatttgtactttactgtttttagttacacgataaccagtaaagacataatttcggttaagtggtggtctaggtttaccaacttcccatgaaagatgtaattctctgtcagacagcacttcacgtacagtagcaataccataagacagtttaaatgtctcctcagtagctttgagcgtttctgctgcaaaaagcttgagtctttcagtacaggtgttagctaaaatgtaatcaccagcatttgtccagtcacatgttgcaattgcattaaagtcagtaacattatcgctaccaacacatgtatttttatataaaccaaaaacttgtccattagcacacaatggaaaactaatgggtggtttatgtgatttacaataatagctcatacctcctaagtaaagttgagtcacatctgtgacatcacaacctggagcattgcaaacatacggattaacagacaagactaatttatgtgatgttgatatgacatggtcgtaacagcatttacaacataagaatggtctacgtatgcaagcaccacatcttaatgaagtctgtgaattgcaaagaacacaagccccaacagcctgtaagactgtatgcggtgtgtacatagcctcataaaactcaggttcccaataccttgaagtgttatcattagtaagcataacagaatacatgtctaacatgtgtcctgttaactcatcatgtagctttcttatgtattgtaagtacaaatgaaagacatcagcatactcctgattaggatgtttagtaagtgggtaagcatctatagctaaagacacgaaccgttcaatcataagtgtaccatctgtttttacgatatcatctacaaaacagccggcccctaggattcttgatggatctgggtaaggaaggtacacataatcatcaccctgtttaactagcattgtatgttgagagcaaaattcatgaggtcctttagtaaggtcagtctcagtccaacattttgcttcagacataaaaacattgttttgataataaagaactgacttaaagttctttatgctagccactagaccttgagatgcataagtgctattgaaacacacaacagcatcgtcagagagtatcatcattgagaaatgtttacgcaaatatgcgtaaaactcattcacaaagtctgtgtcaacatctctatttctatagagacactcataaagtctgtgttgtaaattgcggacatacttatcggcaattttgttaccatcagtagataaaagtgcattaacattggccgtgacagcttgacaaatgttaaaaacactattagcataagcagttgtggcatctcctgatgaggttccacctggtttaacatatagtgaaccgccacacatgaccatttcactcaatacttgagcacactcattagctaatctatagaaacggtgtgacaagctacaacacgttgtatgtttgcgagcaagaacaagtgaggccataattctaagcatgttaggcatggctctatcacatttaggataatcccaacccataaggtgagggttttctacatcactataaacagtttttaacatgttgtgccaaccaccatagaatttgcttgttccaattactacagtagctcctctagtggcggctattgatttcaataatttttgatgaaactgtctattggtcatagtactacagatagagacaccagctacggtgcgagctctattctttgcactaatggcatacttaagattcatttgagttatagtagggatgacattacgttttgtatatgcgaaaagtgcatcttgatcctcataactcattgaatcataataaagtctagccttaccccatttattaaatggaaaaccagctgatttgtctaggttgttgacgatgacttggttagcattaatacagccaccatcgtaacaatcaaagtacttatcaacaacttcaactacaaatagtagttgtctgatatcacacattgttggtagattataacgatagtagtcataatcgctgatagcagcattaccatcctgagcaaagaagaagtgttttaattcaacagaacttccttccttaaagaaacccttagacacagcaaagtcatagaagtctttgttaaaattaccgggtttgacagtttgaaaagcaacattgttagtaagtgcagctactgaaaagcacgtagtgcgtttatctagtaatagattaccagaagcagcgtgcatagcagggtcagcagcatacacaagtaattccttaaaactaagtctagagctatgtaagtttacatcctgattatgtacaacacctagctctctgaagtggtatccagttgaaactacaaatggaacaccatcaacaaatatttttctcactagtggtccaaaacttgtaggtgggaacactgtagagaataaaacattaaagtttgcacaatgcagaatgcatctgtcatccaaacagttaacacaatttgggtggtatgtctgatcccaatatttaaaataacggtcaaagagttttaacctctcttccgtgaagtcatattttaacaaatcccacttaatgtaaggctttgttaagtcagtgtcaacatgtgactctgcagttaaagccctggtcaaggttaatataggcattaacaatgaataataagaatctacaacaggaactccactacctggcgtggtttgtatgaaatcaccgaaatcataccagttaccattgagatcttgattatctaatgtcagtacaccaacaataccagcatttcgcatggcatcacagaattgtactgtttttaacaaagcttggcgtacacgttcacctaagttggcgtatacgcgtaatatatctgggttttctacaaaatcataccagtcctttttattgaaataatcatcatcacaacaattgtatgtgacaagtatttcttttaatgtgtcacaattaccttcatcaaaatgccttaaagcatagacgaggtctgccattgtgtatttagtaagacgttgacgtgatatatgtggtaccatgtcaccgtctattctaaacttaaagaagtcatgtttagcaacagctggacaatccttaagtaaattataaattgtttcttcatgttggtagttagagaaagtgtgtctcttaactacaaagtaagaatcaattaaattgtcatcttcgtccttttcttggaagcgacaacaattagtttttaggaatttagcaaaaccagctactttatcattgtagatgtcaaaagccctgtatacgacatcagtactagtgcctgtgccgcacggtgtaagacgggctgcacttacaccgcaaacccgtttaaaaacgattgtgcatcagctgactgaagcatgggttcgcggagttgatcacaactacagccataacctttccacataccgcagacggtacagactgtgtttttaagtgtaaaacccacagggtcattagcacaagttgtaggtatttgtacatacttaccttttaagtcacaaaatcctttaggatttggatgatctatgtggcaacggcagtacagacaacacgatgcaccaccaaaggattcttgatccatattggcttccggtgtaactgttattgcctgaccagtaccagtgtgtgtacacaacatcttaacacaattagtgattggttgtcccccactagctagataatctttgtaagctttagcagcatctacagcaaaagcacagaaagataatacagttgaattggcaggcacttctgttgcattaccagcttgtagacgtactgtggcagctaaactaccaagtaccatacctctatttaggttgtttaatcctttaataaagtataaatacttcactttaggacctttaggtgtgtctgtaacaaacctacaaggtggttccagttctgtatagatagtaccagttccatcactcttagggaatctagcccatttcaaatcctgtaaatcggataacagtgcaagtacaaacctacctccctttgttgtgttgtagtaagctaacgcattgtcatcagtgcaagcagtttgtgtagtaccggcagcacaagacatctgtcgtagtgcaacaggactaagctcattattctgtaatttgacagcagaattggcccttaaagctgttacaataagaggccatgctaaattaggtgaattgtccatactaatttcactaagttgaacaattttactatctgcatctacaacctgttggatttcccacaatgctgatgcataagtaaatgttgtaccatcacacgtatttttatatgtgttatagtctggtatgacaaccattagtttggctgctgttgtaagaggtattatgttcaagggaacacaaccatctcttgcattgttgataatgttgttgagtgcatcattatccaactttctaagcatagtgaaaagcattgtctgcatagcactagtaacttttgccctcttgtcctcagatctagcctgtttatacatttgggtcatagcttgatcagccatcttttccaacttacgttgcatggctgcatcacggtcaaattcagatttagccacattcaaagacttcttcaactttttaagaacaacttcagaatcaccattagcaacagcctgctcataagcttcttgagcagtagcaaaagctgcatatgatggaagggaactaaactctgaggctatagcttgtaaggttgccctgttgtccagcatttcttcacaaagcttgtttatgtctacagcaccctgcatggaaagcaaaacagaaagtagtgaaaccattttttcaaaggcttcagtagtatctttagctaagagaatgtcattgtgtaactggacacattgagcccacaatttagatgatgattctactctgagttgttgcaaaactgagagtaagactactgatgtgcactttacatctgacattttagactgtacagtggctactttgatacaaggtttgccaccaacacccaacaatttaatgttgagtttgaaggcatctatgctattcttgggtgggagtagtccctgtgaattcatatatctaaactcctgtgtagaaactaagtaatcataaacaccaagagtcagtctaaagtagcggttgagtaaacaaaagaggccaaagtaacaagtacaaaaatagcctaagaaacaataaactagcattatacactgaagtgtattaccagttatgaagaaaatagggcaatactcaacacacataaaaacaatacctctggccaaaaacatgacagttgtaactacacctgagtagttagaagtaacagagattataagagcccacatggaaatggcttgatctaaagcattaccataataaactttataaacgagtgtcaagacattcataagtgtccacactctcctagcaccatcatcatacacagttcttgctgtcataaggattagtaacactacagctgatgcatacataacacagtcttttagcttaaaaccagacaaactagtatcaaccatatccaaccatgtcataatacgcatcacccaactagcaggcatatagaccatattaaaataagctacagtggcaagagaaggtaacaaaaacaaacagagaaatgcatgcttatgtttgacaaacatcattgcaaaagcagacatagcaataatacccatagcaaaaggtaaaaaggcattttcatacaaaaaaaagaacaaagaccattgagtactctggactaaaactaaaagtgaagtcaaaattgtgagtaacaaccagtggtgtgtacccttgattgttcttttcactgcactttggaaagtaacacctgagcattgtctaacaacatcaaaaggtgtaaattcatcttctaataaagcactacccaatatggtacgtccattcataccattttgcagtaattcttttaatgaagcacacatatctaaaacggcaattccagtttgagcagaaagaggtcctagtatgtcaacatggtcttgtgttagaggttcataattgtacttcatagccacaaggttaaagtcattaagagttgtggtaaatcgattgagaaaccacctgtctccatttataacagcagcgtacaaccaagctaaaacattaactgtaatagttgtgtccgtaccagctgcttgtgctgtttgcctgtcaacaaaaggtccataaaagttaccttctaagtctgtgccagcatgaactccagttggtaattccatatggtgcatgtaacaaaaagagacacagtcataatctatgttaaaaccaacactaccacatgaaccattaaggaatgaacccttaatagtgaaattgggcctcatagcacattggtaaacaccagatggtgaaccattgtaacaagctaacactgaaaaagtctgtcctggttgaatgcgaacaaacttatacttaggtgtcttaggattggctgtatcaaccttaagcttaagtacacaattttgcatagaatgtccaataaccctgagttgaacattaccagcctgtaccaagaaattatgattagacttacgaatgagtaaatcttcataattagggttaagcatgtcttcagaggtgcagatcacatgtcttggacagtaaactacgtcatcaagccaaagaccgttaagtgtagttgtaccacaagttacttgtaccatacaaccctcaactttaccagatgggaatgccatttttctaaaaccactctgcaaaacagctgaggtgatagaggtttgtggtggttggtaaagaacatcagaacctgagttactgaagtcattgagagcctttgcgagatgacaacaagcagcttctctgtagctagttgtatccattgctccactaaaatacttgtacttattataaagagctaagtatctattatattgcgtaagaggtaatagcacatcactacgcaactttagatacatttctttatttaacaaaaaggtgcacagcgcagcttcttcaaaagtactaaaggaaacaccattaaagactacacgtctctttaggtaattactaaagaaccaatagaaatgctttgtggaaatacaaatgatataagcaattgttatccagaaaggtactaaaggtgtgaacataaccatccactgaatatgtgctaaaaaagaaacatcattagtaagataaaatgtcaagtacaagtaaataacagaataaacaccaggtaagaatgagtaaactggtgttaaacagagtacagtgaatgacataaggaatagtaaagtattaaaggcaactacatgactgtattcaccaaaagctcttctaaacctcataaaatagtaggcaaggcatgttactacgatagctacaataccaccagctactatagatgctgatatgtccaaagcaccaataggttgaattagtggtgtaaacatattagtaagtaaatttacagcatctacaccacagaaaactcctggtaaagatctgtaataatcattgttaagtacccatctaccactagtagatacacaaacaccagcttctgatctttcacaagtgccgtgcctacagtactcagaatcaaaagttgttaccactctaacagaaccttcaaggtaggtgttaggaaattgaataatagagccatccatgagcacataacgtgtgtcagggcgtaaactttcataagcaacagaaccttctagtacattggtatcataacaatatggtactggcttaccagaagcatctttaaaaattgtacattcagcagccaaaacacaagctgatgttgcaaagtcagtgtactctataagttttgatggtgtgtaacagatgttaccaactgcactaaaaactctaggtaagaaatgcaaaaagtcaccattagttgtgcgtaatatcgtgccaggcaaaccaggcacgacaaaacccacttctcttgttatgactgcagcaatcaatgggcaagctttgtcattagtataactaccaccacgctggctaaaccatgtgtcaaaatcagcatgtttgttagcaaaacaagtatctgtagatgctatgtcacgagtgacaccaccatcaatagccttgtatcctatgatttcacttgaaaagtcagtatgtttagacatgacatgaacaggtgttattaaatagaaaatagcagcaacaaaaaggaacacaagtgtaactttaattaactgcttcaaccaattattaacaattttaccacccttaagtgctatctttgttgttacaacattaacaacttgtctagtagttgcacatgtcaacttaaaaggtaagttattctttttagcagcactacgtatttgttttcgtagttgttcagacaatgacatgaaatctttaacgttccatatcaaagcaatgttgtgactttttgctacctgcgcattaatatgacgcgcactacagtcaatacaagcaccaaggtcacggggtgtcatgttttcaactttgttataggtgagcatatagttattacaactatcgccagtaacttctatgtcagattgatgtgacaatttaagacattcaacaacatctttagtttctacatctgaatcaacaaacccttgccgagctgctgaaataaaagtagataagacattgtctaaggacacattctttgcaagttcagcttctgcagttgcaactagtgttttgagtttttccattggtacgttaaaagttgatgaaaacgtattaacgtaagcatcaaacattttaactgcaacttccgcactatcaccaacatcagacactaatgcctgatctagtaacagtataggttgacacataagctgactgtagtaaacagacgctgattttgcagatgattcttcacattttgatttaccatcaaaaactataacattaataggcaatgaacctttagtgttattagctctcaggttgtctaagttaacaaaatgagagagagaatgtctttcataagtcttttgaccagctttatcaaagtaaagatggatggaaccattcttcactgtaacactatcaacgatgtaagaagactggtcagtaggatttattggtcttttaaactgtagtgacaagtctctcgcaacttcatcactaataaatgtactaccagcacagaatgtatcacaattaacacaattccaattgtgtagtttgcaaaagcctttacctccattagcatagacataaaaggaccttctaacaccattaacaatagttgtacattcgactcttgttgctctattacgtttgtaacacatcatacaagttgatgaattacaaccgtctacaacatgcacataacttttccatacataataaaatgatgcaaagaagatgtacattctaaccatagctgaaatcggggccatttgtacaagattaattattaaccacataagccaagaattactaataaaatgtactgcaaaatagctgaaaaacaattgcatgattgcagccaatccaagtacatagaaaaacctagtgaaaagaatatatgccaaaaaccactctgcaactaagccaaaagcagttaaatcccatttaaaagatgaaatggtaatttgtatagtttctaaagaaggataggtgtctaaagaatctaaaccactaagacaaacactacaaggtatagaaccagtacagtaggttgcaatagtgacattagtagagttcaaatagccttctctgtaaccagtacagtaagaaggcatgcctaaattagacattaaaacacctaaagcagcggttgagtagattaaagaacctaggcaaacacttaatagtaaaaaccaaattataatatttatcagtttagaaaaattaggtgacttcaaataattaaatgaagcctctagacaaaatttaccgacactcttaacagtattctttgctatagtagtcggcatagatgctttaattctagaatttgtacttctagtaaaagtacacaattgtagcaataaagtaaagaaataaggcatataattagtacaaacacggtttaaacaccgtgtaactatgttagtagttgtactaacaactttgttaagaaaaggcttagcataattagctatagtatcccaagggacactattaacagcagctaaaccatgagtagcaagggttttcaaacctaatactctagataattcattaggtttcttaatagtaagactagaattgtctacataagcagccattagatctgtgtggccaacctcttctgtaatttttaaactattatttgctggtttaagtataatgtctcctacaacttcggtagttttcacattacactcaagaacgtctttctgtatggtaggattttccactacttcttcagagactggttttagatcttcgcaggcaagattatccattccctgcgcgtcctctgacttcagtacatcaaacgaatttgatgtttcaactggttttgtgctccaaagacaacgtatacaccaggtatttggtttatacgtggctttattagttgcattgttaacatgccaaacaataggtttatgtaacaatttagctcctttcttaaaagagggtgtgtagtgtttataatcaatagccaccacatcaccatttaagtcagggaaaaatgtaactttaagctctcttgaagcaggtttcttataaccagttaactggtttaaatcatcagcaaatttgatattatcacatacaaacttaaaattatcgaagcttgcgtttggatatggttggtttggtacaagatcaattggttgctctgtgaaataagaattgtctttcttataataattgtccaacttagggtcaatttctgtacaaacaacaccatccaatttataagtaactggttttatggttgttgtgtaactgttttctttgtagaaaacatccgtaataggacctttgtattctgaggactttgtaagtaaagcaccgtctatgcaatacaaagtttctttagaagttatatgtttatagtgaccacactggtaattaccagtgtactcactagcacaagtaaatgtaccatgcttaagttcatactgagcaggtggtgctgacatcataacaaaaggtgactcctgttgtactagatattttgtagcttgtttaccacacgtacaaggtatctgaacacctttcttaaattgttcataagaaagtgtgcccatgtacataacagcttctacacccttaagggttgtctgctgttgtccacaagttttacacaccacgttcaagactcttttgcaagaatctaaattggcatgttgaaacaagtaactcattgtttctctaacatcacctaactcacctactgtcttattacagtaggctaagataagtgcacaaaagttagcagcttcaccagcccttgctctgtaataagcatcttgtagagcaggtggattaaacttcaactctatttgttggagtgttaacaatgcagtggcaagataacagttgttatctgcccatttaatagaagttaaaccattaacttgtgggtatttccactttttagtgtgatttaatgctgacatgtacctacccagaaaactaggatcagttgtgtggtagtactcaaaagcctcaacacgtagagtgtcatcattaggtaaaacataaaatgttttaccttcatgtgaattatgaggttttattttagtaacatcagctccatccaaataagttggaccaaactgttgtccatatgtcattgacatgtccacaacttgcgtgtggaggttaatgttgtctactgttgtaaacaccttaatagtcctcacttctctcaaagaaagaagtgtcttaagattgtcaaaggtgataacttcaccatctaggtggaatgtggtaggattactagtgtaatatacacttttatcacctctcttaagaaattctatacctagttgtgtagattgtccagaataggaccaatctttataggaaccagcaagtgagatggtttcaataaaatgttcttcaggtgttttagaagaagaagtaagataaccattatacgctgtaacagcatcaggtgaagaaacagaaactgtagctggcactttgagagatctcatataccgagcagcttcttccaaatttaagccatgtgttacatagccaagtggcattgtaacaagagtttcatttagatcgttaagtgtgttgataagtgacgctacagttgttttactggtgtaaaagtaaaatctagcaccataatcaaccacaccctcttgtattttaatacccttatatttacgctgtatagttgaaactatggctttagtttccacacagacaggcattaatttgcgtgtttcttctgcatgtgcaagcatttctcgcaaattccaagaaacagttccaagaatttcttgcttctcattagagataatagatggtagaatgtaaaaggcacttttacactttttaagcactgtctttgcctcctctacagtgtaaccatttaaaccctgacccgggtaagtggttatataattgtctgttggcacttttctcaaagctttcgctagcatttcagtagtgccaccagcctttttagtaggtataaccacagcagttaaaacaccctcttgaacaacatcacccactatatatggagcatctttctttaagaaagtgatgtcaatgtcactaacaagagtggcagaatctggatgaagattgccattaatgtcaatataaagtaacaagttttctgtgaggaacttagtttcttccagagttgttgtaacttcttcaacacaagctttgattttcttatcatcttgttttctctgttcaactgaaggtttactttcagttataaatggcttaacttcctctttaggaatctcagcgatcttttgttcaacttgcttttcactcttcatttccaaaaagcttgaaacaagtttgtcatagagatttttatcaaagacagctaagtagacatttgtgcgaacagtatctacacaaactcttaaagaatgtatagggtcagcaccaaaaataccagctgataataatggtgcaagtagaacttcgtgctgattaaaattttcataagcactcttaagaagttgaatgtcttcacctttgttaacatttgggccgacaacatgaagacagtgtttagcaagattgtgtccgcttaaaacacaactaccacccactttaagtggtccattagtagctatgtaatcatcagattcaacttgcatggcattgttagtagccttatttaaggctcctgcaacacctcctccatgtttaaggtaaacattggctgcattaacaaccactgttggttttacctttttagcttcttccacaatgtctgcatttttaatgtatacattgtcagtaagttttaaataaccactaaaactattcacttcaatagtctgaacaactggtgtaagttccatctctaattgaggttgaacctcaacaattgtttgaatagtagttgtctgattgtcctcactgccgtcttgttgaccaacagtttgttgactatcatcatctaaccaatcttcttcttgctcttcttcaggttgaagagcagcagaagtggcaccaaattccaaaggtttaccttggtaatcatcttcagtaccatactcatattgagttgatggctcaaactcttcttcttcacaatcaccttcttcttcatcctcatctggagggtagaaagaacaatacatatgtgaagccaatttaaactcaccagactcatcaaataagtagtatgtagccatactccactcatctaaatcaatgcccagtggtgtaagtaattcagatactggttgcaaagtttttatgacagcatctgccacaacacaggcgaactcatttacttctgtaccgagttcaactgtataggcagagcacttctcattaagtactttatcaatcctttcatcaagttcaaaagtgatattcacactcttgtaaccttgcacttctatcacagtgtcatcaccaaaagtaacctttgttggtgcaccgcctttgagtgtgaaggtattgtttgttaccatcatattaggtgcaagggcacagtacttttctgtgtctttgatttcgagcaacataagcccgttaatacaaactggtgtaccaaccaatggagcttcaacagcttcactagtaggttgttctaatggttgtaaatcaccagttttcaagacaacttcctctgttaacacttctgtgggaagtgtttctccctctaagaagataatttcttttggggcttttagaggcatgagtaggccagtttcttctctggatttaacacactttctgtacaatccctttgagtgcgtgacaaatgtttcacctaaattcaaggctttaagtttagctccaccaataatgatagagtcagcacacaaagccaaaaatttatttacaagcttaaagaatgtctgaacactctccttaatttcctttgcacaggtgacaatttgtccaccgacaatttcacaagcacaggttgagataaatttaacaatttcccaaccgtctctaagaaactctacaccttccttaaacttctcttcaagccaatcaaggacgggtttgagtttttcataaacagtgccaaagatgttagttagccactgcgaagtcaactgaacaacaccacctgtaatgtaggccattacaactagattgttagtagccaaatcagatgtgaacatcatagcatcaatgagtctcagtgaatactgtgaaattccatctagtattgttatagcggccttctgtaaaacacgcacagaattttgagcagtttcaagagtgcgggagaaaattgatcgtacaacacgagcagcctctgatgcaaatgcataaagaggactcagtattgatttctgttcaccaatattccaggcaccttttttagcttttccttttgtaactttaaaattaccacaggattcaacaatttgtttgaatgctttataatccaaacctttcacagtttccacaaaagcacttgtggaagcagaaaaagatgccaaaataatggcgatctcttcattaagtttaaagtcaccaacaatattgatgttgactttctctttttggagtatttcaagaaggttgtcattaagaccttcggaaccttctccaacaacacctgtatggttacaacctatgttagcgctagcacgtggaacccaataggcacacttgttatggcaaccaacataagagaacacacagcctccaaaggcaatagtgcgaccacccttacgaagaatggttttcaagccagattcattatggtattcggcaagactatgctcaggtcctacttctgaattgtgacatgctggacaataaattttaacaacagcattttggggtaagtaaccacaagtagtggcaccttctttagtcaaattctcagtgccacaaaattcgcaagtggctttaacaaaatcgcccgtctgccatgaagtttcaccacaatgatcacacttcatgagagttgaaaggcacatttggttgcattcatttggtgacgcaactggatagacagatcgaattctacccataaagccatcaagctttttcttttcaacccttggttgaatagtcttgattatggaatttaagggaaatacaaaatttggacattccccattgaaggtgtcaaatttctttgccaatttaatttcaaaaggtgtctgcaattcatagctcttttcagaacgttccgtgtaccaagcaatttcatgctcatgttcacggcagcagtatacacccctcttagtgtcaataaagtccagttgttcggacaaagtgcatgaagctttaccagcacgtgctagaaggtctttaatgcactcaagagggtagccatcagggccacagaagttgttatcgacatagcgagtgtatgcccctccgttaagctcacgcatgagttcacgggtaacaccactgctatgtttagtgttccagttttcttgaaaatcttcataaggatcagtgccaagctcgtcgcctaagtcaaatgactttagatcggcgccgtaactatggccaccagctcctttattaccgttcttacgaagaagaaccttgcggtaagccactggtatttcgcccacatgagggacaaggacaccaagtgtctcaccactacgaccgtactgaatgccttcgagttctgctaccagctcaaccataacatgaccatgaggtgcagttcgagcatccgaacgtttgatgaacacatagggctgttcaagttgaggcaaaacgcctttttcaacttctactaagccacaagtgccatctttaagatgttgacgtgcctctgataagacctcctccacggagtctccaaagccacgtacgagcacgtcgcgaacctgtaaaacaggcaaactgagttggacgtgtgttttctcgttgaaaccagggacaaggctctccatcttacctttcggtcacacccggacgaaacctagatgtgctgatgatcggctgcaacacggacgaaaccgtaagcagcctgcagaagatagacgagttactcgtgtcctgtcaacgacagtaattagttattaattatactgcgtgagtgcactaagcatgcagccgagtgacagccacacagattttaaagttcgtttagagaacagatctacaagagatcgaaagttggttggtttgttacctgggaaggtataaacctttaat

SEQ ID NO: 3 - DNA, 16 bp, Artificial sequence
  Synthetic oligonucleotide
gacgaaaccgtaagca

SEQ ID NO: 4 - DNA, 16 bp, Artificial sequence
  Synthetic oligonucleotide
atcgaagcgcagtaag

SEQ ID NO: 5 - DNA, 16 bp, Artificial sequence
  Synthetic oligonucleotide
tgtaagacgggctgca

SEQ ID NO: 6 - DNA, 16 bp, Artificial sequence
  Synthetic oligonucleotide
gtatacactatgcgag

SEQ ID NO: 7 - DNA, 16 bp, Artificial sequence
  Synthetic oligonucleotide
ccgaaacgaatgagta

SEQ ID NO: 8 - DNA, 16 bp, Artificial sequence
  Synthetic oligonucleotide
tccgaaacgaatgagt

SEQ ID NO: 9 - DNA, 16 bp, Artificial sequence
  Synthetic oligonucleotide
gaagtacgctattaac

SEQ ID NO: 10 - DNA, 16 bp, Artificial sequence
  Synthetic oligonucleotide
tcccgaaggtgtgact

SEQ ID NO: 11 - DNA, 16 bp, Artificial sequence
  Synthetic oligonucleotide
cgaaacgaatgagtac

SEQ ID NO: 12 - DNA, 16 bp, Artificial sequence
  Synthetic oligonucleotide
acacaatcgaagcgca

SEQ ID NO: 13 - DNA, 16 bp, Artificial sequence
  Synthetic oligonucleotide
cggtagtagccaattt

SEQ ID NO: 14 - DNA, 16 bp, Artificial sequence
  Synthetic oligonucleotide
tattaccgttcttacg

SEQ ID NO: 15 - DNA, 16 bp, Artificial sequence
  Synthetic oligonucleotide
tatacactatgcgagc

SEQ ID NO: 16 - DNA, 16 bp, Artificial sequence
  Synthetic oligonucleotide
cacacccggacgaaac

SEQ ID NO: 17 - DNA, 16 bp, Artificial sequence
  Synthetic oligonucleotide
tccgaaagggagtgag

SEQ ID NO: 18 - DNA, 16 bp, Artificial sequence
  Synthetic oligonucleotide
cacaatcgaagcgcag

SEQ ID NO: 19 - DNA, 16 bp, Artificial sequence
  Synthetic oligonucleotide
ttcccgaaggtgtgac

SEQ ID NO: 20 - DNA, 16 bp, Artificial sequence
  Synthetic oligonucleotide
ctacaacttcggtagt

SEQ ID NO: 21 - DNA, 16 bp, Artificial sequence
  Synthetic oligonucleotide
cgaaaagtgcatcttg

SEQ ID NO: 22 - DNA, 16 bp, Artificial sequence
  Synthetic oligonucleotide
gcgaaaagtgcatctt

SEQ ID NO: 23 - DNA, 16 bp, Artificial sequence
  Synthetic oligonucleotide
gaaccaagacgcagta

SEQ ID NO: 24 - DNA, 16 bp, Artificial sequence
  Synthetic oligonucleotide
gatctaaagcattacc

SEQ ID NO: 25 - DNA, 16 bp, Artificial sequence
  Synthetic oligonucleotide
ccgcagacggtacaga

SEQ ID NO: 26 - DNA, 16 bp, Artificial sequence
  Synthetic oligonucleotide
aacccgtttaaaaacg

SEQ ID NO: 27 - DNA, 16 bp, Artificial sequence
  Synthetic oligonucleotide
attaccgggtttgaca

SEQ ID NO: 28 - DNA, 16 bp, Artificial sequence
  Synthetic oligonucleotide
acactatgcgagcaga

SEQ ID NO: 29 - DNA, 16 bp, Artificial sequence
  Synthetic oligonucleotide
acgaaaccgtaagcag

SEQ ID NO: 30 - DNA, 16 bp, Artificial sequence
  Synthetic oligonucleotide
atccgaaagggagtga

SEQ ID NO: 31 - DNA, 16 bp, Artificial sequence
  Synthetic oligonucleotide
agggaatctagcccat

SEQ ID NO: 32 - DNA, 16 bp, Artificial sequence
  Synthetic oligonucleotide
aattaccgggtttgac

SEQ ID NO: 33 - DNA, 16 bp, Artificial sequence
  Synthetic oligonucleotide
gcgcagtaaggatggc

SEQ ID NO: 34 - DNA, 16 bp, Artificial sequence
  Synthetic oligonucleotide
accaagacgcagtatt

SEQ ID NO: 35 - DNA, 16 bp, Artificial sequence
  Synthetic oligonucleotide
aaccaagacgcagtat

SEQ ID NO: 36 - DNA, 16 bp, Artificial sequence
  Synthetic oligonucleotide
cgagaagaggcttgac

SEQ ID NO: 37 - DNA, 16 bp, Artificial sequence
  Synthetic oligonucleotide
cccgaaggtgtgactt

SEQ ID NO: 38 - DNA, 16 bp, Artificial sequence
  Synthetic oligonucleotide
cttacaaaggcacgct

SEQ ID NO: 39 - DNA, 16 bp, Artificial sequence
  Synthetic oligonucleotide
aagtacgctattaact

SEQ ID NO: 40 - DNA, 16 bp, Artificial sequence
  Synthetic oligonucleotide
aaattaccgggtttga

SEQ ID NO: 41 - DNA, 16 bp, Artificial sequence
  Synthetic oligonucleotide
tcataagtgtaccatc

SEQ ID NO: 42 - DNA, 16 bp, Artificial sequence
  Synthetic oligonucleotide
ttacaaaggcacgcta

SEQ ID NO: 43 - DNA, 16 bp, Artificial sequence
  Synthetic oligonucleotide
cgctattaactattaa

SEQ ID NO: 44 - DNA, 16 bp, Artificial sequence
  Synthetic oligonucleotide
cgttaacaatattgca

SEQ ID NO: 45 - DNA, 16 bp, Artificial sequence
  Synthetic oligonucleotide
ttaaaattaccgggtt

SEQ ID NO: 46 - DNA, 16 bp, Artificial sequence
  Synthetic oligonucleotide
tttagtaaggtcagtc

SEQ ID NO: 47 - DNA, 16 bp, Artificial sequence
  Synthetic oligonucleotide
gtttaactagcattgt

SEQ ID NO: 48 - DNA, 16 bp, Artificial sequence
  Synthetic oligonucleotide
gaataccacgaaagca

SEQ ID NO: 49 - DNA, 16 bp, Artificial sequence
  Synthetic oligonucleotide
acgcagtattattggg

SEQ ID NO: 50 - DNA, 16 bp, Artificial sequence
  Synthetic oligonucleotide
gttacgagaattcatt

SEQ ID NO: 51 - DNA, 16 bp, Artificial sequence
  Synthetic oligonucleotide
tccatataggcagctc

SEQ ID NO: 52 - DNA, 16 bp, Artificial sequence
  Synthetic oligonucleotide
caacttcggtagtttt

SEQ ID NO: 53 - DNA, 16 bp, Artificial sequence
  Synthetic oligonucleotide
taaaattaccgggttt

SEQ ID NO: 54 - DNA, 16 bp, Artificial sequence
  Synthetic oligonucleotide
gcactaatggcatact

SEQ ID NO: 55 - DNA, 16 bp, Artificial sequence
  Synthetic oligonucleotide
cctttagtaaggtcag

SEQ ID NO: 56 - DNA, 16 bp, Artificial sequence
  Synthetic oligonucleotide
aaagaagtacgctatt

SEQ ID NO: 57 - DNA, 16 bp, Artificial sequence
  Synthetic oligonucleotide
cgagaattcattctgc

SEQ ID NO: 58 - DNA, 16 bp, Artificial sequence
  Synthetic oligonucleotide
tagttacgagaattca

SEQ ID NO: 59 - DNA, 16 bp, Artificial sequence
  Synthetic oligonucleotide
acattagggctcttcc

SEQ ID NO: 60 - DNA, 16 bp, Artificial sequence
  Synthetic oligonucleotide
ctattagcataagcag

SEQ ID NO: 61 - DNA, 16 bp, Artificial sequence
  Synthetic oligonucleotide
cactattagcataagc

SEQ ID NO: 62 - DNA, 16 bp, Artificial sequence
  Synthetic oligonucleotide
tacaaaggcacgctag

SEQ ID NO: 63 - DNA, 16 bp, Artificial sequence
  Synthetic oligonucleotide
aagaagtacgctatta

SEQ ID NO: 64 - DNA, 16 bp, Artificial sequence
  Synthetic oligonucleotide
ttatagtagggatgac

SEQ ID NO: 65 - DNA, 16 bp, Artificial sequence
  Synthetic oligonucleotide
gagttatagtagggat

SEQ ID NO: 66 - DNA, 16 bp, Artificial sequence
  Synthetic oligonucleotide
gataatcccaacccat

SEQ ID NO: 67 - DNA, 16 bp, Artificial sequence
  Synthetic oligonucleotide
tagtaaggtcagtctc

SEQ ID NO: 68 - DNA, 16 bp, Artificial sequence
  Synthetic oligonucleotide
cataagtgtaccatct

SEQ ID NO: 69 - DNA, 16 bp, Artificial sequence
  Synthetic oligonucleotide
attaagatctgaatcg

SEQ ID NO: 70 - DNA, 16 bp, Artificial sequence
  Synthetic oligonucleotide
acgctattaactatta

SEQ ID NO: 71 - DNA, 16 bp, Artificial sequence
  Synthetic oligonucleotide
gaaaaagaagtacgct

SEQ ID NO: 72 - DNA, 16 bp, Artificial sequence
  Synthetic oligonucleotide
tattactaggttccat

SEQ ID NO: 73 - DNA, 16 bp, Artificial sequence
  Synthetic oligonucleotide
acacatagggctgttc

SEQ ID NO: 74 - DNA, 16 bp, Artificial sequence
  Synthetic oligonucleotide
ccatacctctatttag

SEQ ID NO: 75 - DNA, 16 bp, Artificial sequence
  Synthetic oligonucleotide
tagcataagcagttgt

SEQ ID NO: 76 - DNA, 16 bp, Artificial sequence
  Synthetic oligonucleotide
caatcataagtgtacc

SEQ ID NO: 77 - DNA, 16 bp, Artificial sequence
  Synthetic oligonucleotide
aaaagaagtacgctat

SEQ ID NO: 78 - DNA, 16 bp, Artificial sequence
  Synthetic oligonucleotide
agaataccacgaaagc

SEQ ID NO: 79 - DNA, 16 bp, Artificial sequence
  Synthetic oligonucleotide
ctattactaggttcca

SEQ ID NO: 80 - DNA, 16 bp, Artificial sequence
  Synthetic oligonucleotide
acctacaaggtggttc

SEQ ID NO: 81 - DNA, 16 bp, Artificial sequence
  Synthetic oligonucleotide
gatatatgtggtacca

SEQ ID NO: 82 - DNA, 16 bp, Artificial sequence
  Synthetic oligonucleotide
caaaacttgtaggtgg

SEQ ID NO: 83 - DNA, 16 bp, Artificial sequence
  Synthetic oligonucleotide
ttctatagagacactc

SEQ ID NO: 84 - DNA, 16 bp, Artificial sequence
  Synthetic oligonucleotide
cgaaagggagtgaggc

SEQ ID NO: 85 - DNA, 16 bp, Artificial sequence
  Synthetic oligonucleotide
tactaggttccattgt

SEQ ID NO: 86 - DNA, 16 bp, Artificial sequence
  Synthetic oligonucleotide
acctattactaggttc

SEQ ID NO: 87 - DNA, 16 bp, Artificial sequence
  Synthetic oligonucleotide
gaaacctattactagg

SEQ ID NO: 88 - DNA, 16 bp, Artificial sequence
  Synthetic oligonucleotide
tacgagaattcattct

SEQ ID NO: 89 - DNA, 16 bp, Artificial sequence
  Synthetic oligonucleotide
cacattagggctcttc

SEQ ID NO: 90 - DNA, 16 bp, Artificial sequence
  Synthetic oligonucleotide
ggcaaactgagttgga

SEQ ID NO: 91 - DNA, 16 bp, Artificial sequence
  Synthetic oligonucleotide
cctacaaggtggttcc

SEQ ID NO: 92 - DNA, 16 bp, Artificial sequence
  Synthetic oligonucleotide
aaaattaccgggtttg

SEQ ID NO: 93 - DNA, 16 bp, Artificial sequence
  Synthetic oligonucleotide
aatacagccaccatcg

SEQ ID NO: 94 - DNA, 16 bp, Artificial sequence
  Synthetic oligonucleotide
ttagtaaggtcagtct

SEQ ID NO: 95 - DNA, 16 bp, Artificial sequence
  Synthetic oligonucleotide
ctttagtaaggtcagt

SEQ ID NO: 96 - DNA, 16 bp, Artificial sequence
  Synthetic oligonucleotide
cgcttaacaaagcact

SEQ ID NO: 97 - DNA, 16 bp, Artificial sequence
  Synthetic oligonucleotide
aaatctccataaacga

SEQ ID NO: 98 - DNA, 16 bp, Artificial sequence
  Synthetic oligonucleotide
acaaaggcacgctagt

SEQ ID NO: 99 - DNA, 16 bp, Artificial sequence
  Synthetic oligonucleotide
tagtagccaatttggt

SEQ ID NO: 100 - DNA, 16 bp, Artificial sequence
  Synthetic oligonucleotide
ttacgagaattcattc

SEQ ID NO: 101 - DNA, 16 bp, Artificial sequence
  Synthetic oligonucleotide
agttacgagaattcat

SEQ ID NO: 102 - DNA, 16 bp, Artificial sequence
  Synthetic oligonucleotide
ccatataggcagctct

SEQ ID NO: 103 - DNA, 16 bp, Artificial sequence
  Synthetic oligonucleotide
acacattagggctctt

SEQ ID NO: 104 - DNA, 16 bp, Artificial sequence
  Synthetic oligonucleotide
ttttacacattagggc

SEQ ID NO: 105 - DNA, 16 bp, Artificial sequence
  Synthetic oligonucleotide
gcaaactgagttggac

SEQ ID NO: 106 - DNA, 16 bp, Artificial sequence
  Synthetic oligonucleotide
tcttagggaatctagc

SEQ ID NO: 107 - DNA, 16 bp, Artificial sequence
  Synthetic oligonucleotide
tacaaggtggttccag

SEQ ID NO: 108 - DNA, 16 bp, Artificial sequence
  Synthetic oligonucleotide
ctacaaggtggttcca

SEQ ID NO: 109 - DNA, 16 bp, Artificial sequence
  Synthetic oligonucleotide
cagtactagtgcctgt

SEQ ID NO: 110 - DNA, 16 bp, Artificial sequence
  Synthetic oligonucleotide
ccaaaacttgtaggtg

SEQ ID NO: 111 - DNA, 16 bp, Artificial sequence
  Synthetic oligonucleotide
taaagtctagccttac

SEQ ID NO: 112 - DNA, 16 bp, Artificial sequence
  Synthetic oligonucleotide
tcccaacccataaggt

SEQ ID NO: 113 - DNA, 16 bp, Artificial sequence
  Synthetic oligonucleotide
cttaccagtaccaggt

SEQ ID NO: 114 - DNA, 16 bp, Artificial sequence
  Synthetic oligonucleotide
gcaaacccgtttaaaa

SEQ ID NO: 115 - DNA, 16 bp, Artificial sequence
  Synthetic oligonucleotide
tatctaatgtcagtac

SEQ ID NO: 116 - DNA, 16 bp, Artificial sequence
  Synthetic oligonucleotide
gtccaaaacttgtagg

SEQ ID NO: 117 - DNA, 16 bp, Artificial sequence
  Synthetic oligonucleotide
ataataaagtctagcc

SEQ ID NO: 118 - DNA, 16 bp, Artificial sequence
  Synthetic oligonucleotide
gtacatagcctcataa

SEQ ID NO: 119 - DNA, 16 bp, Artificial sequence
  Synthetic oligonucleotide
ccgaaagggagtgagg

SEQ ID NO: 120 - DNA, 16 bp, Artificial sequence
  Synthetic oligonucleotide
aatattgcagcagtac

SEQ ID NO: 121 - DNA, 16 bp, Artificial sequence
  Synthetic oligonucleotide
gaaatggtgaattgcc

SEQ ID NO: 122 - DNA, 16 bp, Artificial sequence
  Synthetic oligonucleotide
gaggaagttgtagcac

SEQ ID NO: 123 - DNA, 16 bp, Artificial sequence
  Synthetic oligonucleotide
ctcttagggaatctag

SEQ ID NO: 124 - DNA, 16 bp, Artificial sequence
  Synthetic oligonucleotide
taactagcattgtatg

SEQ ID NO: 125 - DNA, 16 bp, Artificial sequence
  Synthetic oligonucleotide
caagattagcagaagc

SEQ ID NO: 126 - DNA, 16 bp, Artificial sequence
  Synthetic oligonucleotide
taccacgaaagcaaga

SEQ ID NO: 127 - DNA, 16 bp, Artificial sequence
  Synthetic oligonucleotide
attattgggtaaacct

SEQ ID NO: 128 - DNA, 16 bp, Artificial sequence
  Synthetic oligonucleotide
cagtattattgggtaa

SEQ ID NO: 129 - DNA, 16 bp, Artificial sequence
  Synthetic oligonucleotide
ttccatataggcagct

SEQ ID NO: 130 - DNA, 16 bp, Artificial sequence
  Synthetic oligonucleotide
gaacacatagggctgt

SEQ ID NO: 131 - DNA, 16 bp, Artificial sequence
  Synthetic oligonucleotide
caccagatggtgaacc

SEQ ID NO: 132 - DNA, 16 bp, Artificial sequence
  Synthetic oligonucleotide
aacctacaaggtggtt

SEQ ID NO: 133 - DNA, 16 bp, Artificial sequence
  Synthetic oligonucleotide
cactaatggcatactt

SEQ ID NO: 134 - DNA, 16 bp, Artificial sequence
  Synthetic oligonucleotide
gcaaaatgactcttac

SEQ ID NO: 135 - DNA, 16 bp, Artificial sequence
  Synthetic oligonucleotide
cccaaattattgagta

SEQ ID NO: 136 - DNA, 16 bp, Artificial sequence
  Synthetic oligonucleotide
agaaaaagaagtacgc

SEQ ID NO: 137 - DNA, 16 bp, Artificial sequence
  Synthetic oligonucleotide
aataccacgaaagcaa

SEQ ID NO: 138 - DNA, 16 bp, Artificial sequence
  Synthetic oligonucleotide
agtaaggatggctagt

SEQ ID NO: 139 - DNA, 16 bp, Artificial sequence
  Synthetic oligonucleotide
aggaaacctattacta

SEQ ID NO: 140 - DNA, 16 bp, Artificial sequence
  Synthetic oligonucleotide
cacaagagtagactat

SEQ ID NO: 141 - DNA, 16 bp, Artificial sequence
  Synthetic oligonucleotide
gcattaatacagccac

SEQ ID NO: 142 - DNA, 16 bp, Artificial sequence
  Synthetic oligonucleotide
taataaagtctagcct

SEQ ID NO: 143 - DNA, 16 bp, Artificial sequence
  Synthetic oligonucleotide
atcataagtgtaccat

SEQ ID NO: 144 - DNA, 16 bp, Artificial sequence
  Synthetic oligonucleotide
atcatagagatgagtc

SEQ ID NO: 145 - DNA, 16 bp, Artificial sequence
  Synthetic oligonucleotide
aggttataagtatcaa

SEQ ID NO: 146 - DNA, 16 bp, Artificial sequence
  Synthetic oligonucleotide
ctattaactattaacg

SEQ ID NO: 147 - DNA, 16 bp, Artificial sequence
  Synthetic oligonucleotide
cagtaaggatggctag

SEQ ID NO: 148 - DNA, 16 bp, Artificial sequence
  Synthetic oligonucleotide
cattaatacagccacc

SEQ ID NO: 149 - DNA, 16 bp, Artificial sequence
  Synthetic oligonucleotide
agttatagtagggatg

SEQ ID NO: 150 - DNA, 16 bp, Artificial sequence
  Synthetic oligonucleotide
atagtactacagatag

SEQ ID NO: 151 - DNA, 16 bp, Artificial sequence
  Synthetic oligonucleotide
tcatagtactacagat

SEQ ID NO: 152 - DNA, 16 bp, Artificial sequence
  Synthetic oligonucleotide
actattagcataagca

SEQ ID NO: 153 - DNA, 16 bp, Artificial sequence
  Synthetic oligonucleotide
tctatagagacactca

SEQ ID NO: 154 - DNA, 16 bp, Artificial sequence
  Synthetic oligonucleotide
taccagtaccaggtgg

SEQ ID NO: 155 - DNA, 16 bp, Artificial sequence
  Synthetic oligonucleotide
tacgctattaactatt

SEQ ID NO: 156 - DNA, 16 bp, Artificial sequence
  Synthetic oligonucleotide
aacctattactaggtt

SEQ ID NO: 157 - DNA, 16 bp, Artificial sequence
  Synthetic oligonucleotide
agccaatttggtcatc

SEQ ID NO: 158 - DNA, 16 bp, Artificial sequence
  Synthetic oligonucleotide
ttcttacgaagaagaa

SEQ ID NO: 159 - DNA, 16 bp, Artificial sequence
  Synthetic oligonucleotide
ccacaaggttaaagtc

SEQ ID NO: 160 - DNA, 16 bp, Artificial sequence
  Synthetic oligonucleotide
acatttgggtcatagc

SEQ ID NO: 161 - DNA, 16 bp, Artificial sequence
  Synthetic oligonucleotide
ctctatttaggttgtt

SEQ ID NO: 162 - DNA, 16 bp, Artificial sequence
  Synthetic oligonucleotide
cataataaagtctagc

SEQ ID NO: 163 - DNA, 16 bp, Artificial sequence
  Synthetic oligonucleotide
tcataaagtaatgggt

SEQ ID NO: 164 - DNA, 16 bp, Artificial sequence
  Synthetic oligonucleotide
gttaacaatattgcag

SEQ ID NO: 165 - DNA, 16 bp, Artificial sequence
  Synthetic oligonucleotide
tattattgggtaaacc

SEQ ID NO: 166 - DNA, 16 bp, Artificial sequence
  Synthetic oligonucleotide
aactacagccataacc

SEQ ID NO: 167 - DNA, 16 bp, Artificial sequence
  Synthetic oligonucleotide
gtactacagatagaga

SEQ ID NO: 168 - DNA, 16 bp, Artificial sequence
  Synthetic oligonucleotide
agtaaggtcagtctca

SEQ ID NO: 169 - DNA, 16 bp, Artificial sequence
  Synthetic oligonucleotide
agctaagttgtttaac

SEQ ID NO: 170 - DNA, 16 bp, Artificial sequence
  Synthetic oligonucleotide
gagctaagttgtttaa

SEQ ID NO: 171 - DNA, 16 bp, Artificial sequence
  Synthetic oligonucleotide
gcagtaaggatggcta

SEQ ID NO: 172 - DNA, 16 bp, Artificial sequence
  Synthetic oligonucleotide
gtattattgggtaaac

SEQ ID NO: 173 - DNA, 16 bp, Artificial sequence
  Synthetic oligonucleotide
catataggcagctctc

SEQ ID NO: 174 - DNA, 16 bp, Artificial sequence
  Synthetic oligonucleotide
acatagggctgttcaa

SEQ ID NO: 175 - DNA, 16 bp, Artificial sequence
  Synthetic oligonucleotide
taaacaccagatggtg

SEQ ID NO: 176 - DNA, 16 bp, Artificial sequence
  Synthetic oligonucleotide
tgatctaaagcattac

SEQ ID NO: 177 - DNA, 16 bp, Artificial sequence
  Synthetic oligonucleotide
cactaagttgaacaat

SEQ ID NO: 178 - DNA, 16 bp, Artificial sequence
  Synthetic oligonucleotide
cctttaggatttggat

SEQ ID NO: 179 - DNA, 16 bp, Artificial sequence
  Synthetic oligonucleotide
atatatgtggtaccat

SEQ ID NO: 180 - DNA, 16 bp, Artificial sequence
  Synthetic oligonucleotide
tagtactacagataga

SEQ ID NO: 181 - DNA, 16 bp, Artificial sequence
  Synthetic oligonucleotide
gagaaatgtttacgca

SEQ ID NO: 182 - DNA, 16 bp, Artificial sequence
  Synthetic oligonucleotide
tatcagacattatgca

SEQ ID NO: 183 - DNA, 16 bp, Artificial sequence
  Synthetic oligonucleotide
aaacccatcatagaga

SEQ ID NO: 184 - DNA, 16 bp, Artificial sequence
  Synthetic oligonucleotide
caacactaccacatga

SEQ ID NO: 185 - DNA, 16 bp, Artificial sequence
  Synthetic oligonucleotide
gagctatgtaagttta

SEQ ID NO: 186 - DNA, 16 bp, Artificial sequence
  Synthetic oligonucleotide
agtactacagatagag

SEQ ID NO: 187 - DNA, 16 bp, Artificial sequence
  Synthetic oligonucleotide
ctatagagacactcat

SEQ ID NO: 188 - DNA, 16 bp, Artificial sequence
  Synthetic oligonucleotide
caataatagctcatac

SEQ ID NO: 189 - DNA, 16 bp, Artificial sequence
  Synthetic oligonucleotide
ctctatcagacattat

SEQ ID NO: 190 - DNA, 16 bp, Artificial sequence
  Synthetic oligonucleotide
gttattagagtaagca

SEQ ID NO: 191 - DNA, 16 bp, Artificial sequence
  Synthetic oligonucleotide
ctttttgattgtccaa

SEQ ID NO: 192 - DNA, 16 bp, Artificial sequence
  Synthetic oligonucleotide
caagaataccacgaaa

SEQ ID NO: 193 - DNA, 16 bp, Artificial sequence
  Synthetic oligonucleotide
attactaggttccatt

SEQ ID NO: 194 - DNA, 16 bp, Artificial sequence
  Synthetic oligonucleotide
ccattaacaatagttg

SEQ ID NO: 195 - DNA, 16 bp, Artificial sequence
  Synthetic oligonucleotide
caaacctacaaggtgg

SEQ ID NO: 196 - DNA, 16 bp, Artificial sequence
  Synthetic oligonucleotide
caactacagccataac

SEQ ID NO: 197 - DNA, 16 bp, Artificial sequence
  Synthetic oligonucleotide
caacaacttcaactac

SEQ ID NO: 198 - DNA, 16 bp, Artificial sequence
  Synthetic oligonucleotide
atagagacaccagcta

SEQ ID NO: 199 - DNA, 16 bp, Artificial sequence
  Synthetic oligonucleotide
acactattagcataag

SEQ ID NO: 200 - DNA, 16 bp, Artificial sequence
  Synthetic oligonucleotide
ttaactagcattgtat

SEQ ID NO: 201 - DNA, 16 bp, Artificial sequence
  Synthetic oligonucleotide
acccaaattattgagt

SEQ ID NO: 202 - DNA, 16 bp, Artificial sequence
  Synthetic oligonucleotide
ctccaattaatgtgac

SEQ ID NO: 203 - DNA, 16 bp, Artificial sequence
  Synthetic oligonucleotide
gctaagttgtttaaca

SEQ ID NO: 204 - DNA, 16 bp, Artificial sequence
  Synthetic oligonucleotide
aagaataccacgaaag

SEQ ID NO: 205 - DNA, 16 bp, Artificial sequence
  Synthetic oligonucleotide
acattttagactgtac

SEQ ID NO: 206 - DNA, 16 bp, Artificial sequence
  Synthetic oligonucleotide
aaacccgtttaaaaac

SEQ ID NO: 207 - DNA, 16 bp, Artificial sequence
  Synthetic oligonucleotide
agctatgtaagtttac

SEQ ID NO: 208 - DNA, 16 bp, Artificial sequence
  Synthetic oligonucleotide
tactacagatagagac

SEQ ID NO: 209 - DNA, 16 bp, Artificial sequence
  Synthetic oligonucleotide
tcatagagatgagtct

SEQ ID NO: 210 - DNA, 16 bp, Artificial sequence
  Synthetic oligonucleotide
gcccaaagctcaaatg

SEQ ID NO: 211 - DNA, 16 bp, Artificial sequence
  Synthetic oligonucleotide
cattaagatctgaatc

SEQ ID NO: 212 - DNA, 16 bp, Artificial sequence
  Synthetic oligonucleotide
aacgaatgagtacata

SEQ ID NO: 213 - DNA, 16 bp, Artificial sequence
  Synthetic oligonucleotide
gcaaagcaatattgtc

SEQ ID NO: 214 - DNA, 16 bp, Artificial sequence
  Synthetic oligonucleotide
gcacaagagtagacta

SEQ ID NO: 215 - DNA, 16 bp, Artificial sequence
  Synthetic oligonucleotide
aacacatagggctgtt

SEQ ID NO: 216 - DNA, 16 bp, Artificial sequence
  Synthetic oligonucleotide
atcctttaggatttgg

SEQ ID NO: 217 - DNA, 16 bp, Artificial sequence
  Synthetic oligonucleotide
tatagagacactcata

SEQ ID NO: 218 - DNA, 16 bp, Artificial sequence
  Synthetic oligonucleotide
cctgattaggatgttt

SEQ ID NO: 219 - DNA, 16 bp, Artificial sequence
  Synthetic oligonucleotide
gcaagattagcagaag

SEQ ID NO: 220 - DNA, 16 bp, Artificial sequence
  Synthetic oligonucleotide
caatattgcagcagta

SEQ ID NO: 221 - DNA, 16 bp, Artificial sequence
  Synthetic oligonucleotide
atataggcagctctcc

SEQ ID NO: 222 - DNA, 16 bp, Artificial sequence
  Synthetic oligonucleotide
ctaaagcattaccata

SEQ ID NO: 223 - DNA, 16 bp, Artificial sequence
  Synthetic oligonucleotide
aaacctacaaggtggt

SEQ ID NO: 224 - DNA, 16 bp, Artificial sequence
  Synthetic oligonucleotide
actaatggcatactta

SEQ ID NO: 225 - DNA, 16 bp, Artificial sequence
  Synthetic oligonucleotide
tattagcataagcagt

SEQ ID NO: 226 - DNA, 16 bp, Artificial sequence
  Synthetic oligonucleotide
cccaaagctcaaatgc

SEQ ID NO: 227 - DNA, 16 bp, Artificial sequence
  Synthetic oligonucleotide
acacttgaaattgcac

SEQ ID NO: 228 - DNA, 16 bp, Artificial sequence
  Synthetic oligonucleotide
tgaaatggtgaattgc

SEQ ID NO: 229 - DNA, 16 bp, Artificial sequence
  Synthetic oligonucleotide
taaagattgctatgtg

SEQ ID NO: 230 - DNA, 16 bp, Artificial sequence
  Synthetic oligonucleotide
gattaaagattgctat

SEQ ID NO: 231 - DNA, 16 bp, Artificial sequence
  Synthetic oligonucleotide
aacaccagatggtgaa

SEQ ID NO: 232 - DNA, 16 bp, Artificial sequence
  Synthetic oligonucleotide
ttaaaaccaacactac

SEQ ID NO: 233 - DNA, 16 bp, Artificial sequence
  Synthetic oligonucleotide
tttaactagcattgta

SEQ ID NO: 234 - DNA, 16 bp, Artificial sequence
  Synthetic oligonucleotide
gcttaacaaagcactc

SEQ ID NO: 235 - DNA, 16 bp, Artificial sequence
  Synthetic oligonucleotide
aaaaagaagtacgcta

SEQ ID NO: 236 - DNA, 16 bp, Artificial sequence
  Synthetic oligonucleotide
tccctaatgttacaca

SEQ ID NO: 237 - DNA, 16 bp, Artificial sequence
  Synthetic oligonucleotide
acccgcgaagaagcta

SEQ ID NO: 238 - DNA, 16 bp, Artificial sequence
  Synthetic oligonucleotide
cacccgcgaagaagct

SEQ ID NO: 239 - DNA, 16 bp, Artificial sequence
  Synthetic oligonucleotide
ctaccgaagagctacc

SEQ ID NO: 240 - DNA, 16 bp, Artificial sequence
  Synthetic oligonucleotide
taaacgggtttgcggt

SEQ ID NO: 241 - DNA, 16 bp, Artificial sequence
  Synthetic oligonucleotide
tttaaacgggtttgcg

SEQ ID NO: 242 - DNA, 16 bp, Artificial sequence
  Synthetic oligonucleotide
actaccgaagttgtag

SEQ ID NO: 243 - DNA, 16 bp, Artificial sequence
  Synthetic oligonucleotide
aaacgggtttgcggtg

SEQ ID NO: 244 - DNA, 16 bp, Artificial sequence
  Synthetic oligonucleotide
ttaaacgggtttgcgg

SEQ ID NO: 245 - DNA, 16 bp, Artificial sequence
  Synthetic oligonucleotide
ctaccgaagttgtagg

SEQ ID NO: 246 - DNA, 16 bp, Artificial sequence
  Synthetic oligonucleotide
aactaccgaagttgta

SEQ ID NO: 247 - DNA, 16 bp, Artificial sequence
  Synthetic oligonucleotide
cccgcgaagaagctat

SEQ ID NO: 248 - DNA, 16 bp, Artificial sequence
  Synthetic oligonucleotide
acccaataatactgcg

SEQ ID NO: 249 - DNA, 16 bp, Artificial sequence
  Synthetic oligonucleotide
cgtaagaacggtaata

SEQ ID NO: 250 - DNA, 16 bp, Artificial sequence
  Synthetic oligonucleotide
aaagaaattgaccgcc

SEQ ID NO: 251 - DNA, 16 bp, Artificial sequence
  Synthetic oligonucleotide
caaacattggccgcaa

SEQ ID NO: 252 - DNA, 16 bp, Artificial sequence
  Synthetic oligonucleotide
actaccgaagagctac

SEQ ID NO: 253 - DNA, 16 bp, Artificial sequence
  Synthetic oligonucleotide
cgtttttaaacgggtt

SEQ ID NO: 254 - DNA, 16 bp, Artificial sequence
  Synthetic oligonucleotide
aacatacgagggcaat

SEQ ID NO: 255 - DNA, 16 bp, Artificial sequence
  Synthetic oligonucleotide
gcgcaaacaggttcat

SEQ ID NO: 256 - DNA, 16 bp, Artificial sequence
  Synthetic oligonucleotide
caaaaggcttctacgc

SEQ ID NO: 257 - DNA, 16 bp, Artificial sequence
  Synthetic oligonucleotide
caaagaatagagctcg

SEQ ID NO: 258 - DNA, 16 bp, Artificial sequence
  Synthetic oligonucleotide
catacgagggcaattc

SEQ ID NO: 259 - DNA, 16 bp, Artificial sequence
  Synthetic oligonucleotide
aaattgaccgcctcaa

SEQ ID NO: 260 - DNA, 16 bp, Artificial sequence
  Synthetic oligonucleotide
gaaattgaccgcctca

SEQ ID NO: 261 - DNA, 16 bp, Artificial sequence
  Synthetic oligonucleotide
ttcataacagatgcgc

SEQ ID NO: 262 - DNA, 16 bp, Artificial sequence
  Synthetic oligonucleotide
gaatagagctcgcacc

SEQ ID NO: 263 - DNA, 16 bp, Artificial sequence
  Synthetic oligonucleotide
agaatagagctcgcac

SEQ ID NO: 264 - DNA, 16 bp, Artificial sequence
  Synthetic oligonucleotide
tacgagggcaattcac

SEQ ID NO: 265 - DNA, 16 bp, Artificial sequence
  Synthetic oligonucleotide
gcctatatggaagagc

SEQ ID NO: 266 - DNA, 16 bp, Artificial sequence
  Synthetic oligonucleotide
cgcaaacaggttcatc

SEQ ID NO: 267 - DNA, 16 bp, Artificial sequence
  Synthetic oligonucleotide
tactaccgaagagcta

SEQ ID NO: 268 - DNA, 16 bp, Artificial sequence
  Synthetic oligonucleotide
gccactagaggagcta

SEQ ID NO: 269 - DNA, 16 bp, Artificial sequence
  Synthetic oligonucleotide
tcaaacccggtaattt

SEQ ID NO: 270 - DNA, 16 bp, Artificial sequence
  Synthetic oligonucleotide
tagataaacgcactac

SEQ ID NO: 271 - DNA, 16 bp, Artificial sequence
  Synthetic oligonucleotide
ttttaaacgggtttgc

SEQ ID NO: 272 - DNA, 16 bp, Artificial sequence
  Synthetic oligonucleotide
gcactacgacagatgt

SEQ ID NO: 273 - DNA, 16 bp, Artificial sequence
  Synthetic oligonucleotide
acatacgagggcaatt

SEQ ID NO: 274 - DNA, 16 bp, Artificial sequence
  Synthetic oligonucleotide
agaaattgaccgcctc

SEQ ID NO: 275 - DNA, 16 bp, Artificial sequence
  Synthetic oligonucleotide
tcataacagatgcgca

SEQ ID NO: 276 - DNA, 16 bp, Artificial sequence
  Synthetic oligonucleotide
ctactaccgaagagct

SEQ ID NO: 277 - DNA, 16 bp, Artificial sequence
  Synthetic oligonucleotide
caaattggctactacc

SEQ ID NO: 278 - DNA, 16 bp, Artificial sequence
  Synthetic oligonucleotide
ccaaattggctactac

SEQ ID NO: 279 - DNA, 16 bp, Artificial sequence
  Synthetic oligonucleotide
caaactgtcaaacccg

SEQ ID NO: 280 - DNA, 16 bp, Artificial sequence
  Synthetic oligonucleotide
ctagataaacgcacta

SEQ ID NO: 281 - DNA, 16 bp, Artificial sequence
  Synthetic oligonucleotide
cactacgacagatgtc

SEQ ID NO: 282 - DNA, 16 bp, Artificial sequence
  Synthetic oligonucleotide
atacgagggcaattca

SEQ ID NO: 283 - DNA, 16 bp, Artificial sequence
  Synthetic oligonucleotide
gaacatacgagggcaa

SEQ ID NO: 284 - DNA, 16 bp, Artificial sequence
  Synthetic oligonucleotide
cgaaaggtaagatgga

SEQ ID NO: 285 - DNA, 16 bp, Artificial sequence
  Synthetic oligonucleotide
aagaaattgaccgcct

SEQ ID NO: 286 - DNA, 16 bp, Artificial sequence
  Synthetic oligonucleotide
cgcaaattgcacaatt

SEQ ID NO: 287 - DNA, 16 bp, Artificial sequence
  Synthetic oligonucleotide
ccgcaaattgcacaat

SEQ ID NO: 288 - DNA, 16 bp, Artificial sequence
  Synthetic oligonucleotide
cctatatggaagagcc

SEQ ID NO: 289 - DNA, 16 bp, Artificial sequence
  Synthetic oligonucleotide
ccaaaaggcttctacg

SEQ ID NO: 290 - DNA, 16 bp, Artificial sequence
  Synthetic oligonucleotide
ccactagaggagctac

SEQ ID NO: 291 - DNA, 16 bp, Artificial sequence
  Synthetic oligonucleotide
aagaatagagctcgca

SEQ ID NO: 292 - DNA, 16 bp, Artificial sequence
  Synthetic oligonucleotide
taccacatatatcacg

SEQ ID NO: 293 - DNA, 16 bp, Artificial sequence
  Synthetic oligonucleotide
aacgagaaaacacacg

SEQ ID NO: 294 - DNA, 16 bp, Artificial sequence
  Synthetic oligonucleotide
cactagaggagctact

SEQ ID NO: 295 - DNA, 16 bp, Artificial sequence
  Synthetic oligonucleotide
ttaatagttaatagcg

SEQ ID NO: 296 - DNA, 16 bp, Artificial sequence
  Synthetic oligonucleotide
tacaaacattggccgc

SEQ ID NO: 297 - DNA, 16 bp, Artificial sequence
  Synthetic oligonucleotide
ctatatggaagagccc

SEQ ID NO: 298 - DNA, 16 bp, Artificial sequence
  Synthetic oligonucleotide
aatgatgaatgtcgca

SEQ ID NO: 299 - DNA, 16 bp, Artificial sequence
  Synthetic oligonucleotide
taatgatgaatgtcgc

SEQ ID NO: 300 - DNA, 16 bp, Artificial sequence
  Synthetic oligonucleotide
cccaaggtttacccaa

SEQ ID NO: 301 - DNA, 16 bp, Artificial sequence
  Synthetic oligonucleotide
aaagaatagagctcgc

SEQ ID NO: 302 - DNA, 16 bp, Artificial sequence
  Synthetic oligonucleotide
caagatgcacttttcg

SEQ ID NO: 303 - DNA, 16 bp, Artificial sequence
  Synthetic oligonucleotide
agttttggaccactag

SEQ ID NO: 304 - DNA, 16 bp, Artificial sequence
  Synthetic oligonucleotide
aaaactaccgaagttg

SEQ ID NO: 305 - DNA, 16 bp, Artificial sequence
  Synthetic oligonucleotide
gcagaatgaattctcg

SEQ ID NO: 306 - DNA, 16 bp, Artificial sequence
  Synthetic oligonucleotide
ctagtaataggtttcc

SEQ ID NO: 307 - DNA, 16 bp, Artificial sequence
  Synthetic oligonucleotide
aaaagaaattgaccgc

SEQ ID NO: 308 - DNA, 16 bp, Artificial sequence
  Synthetic oligonucleotide
gaatcttaagtatgcc

SEQ ID NO: 309 - DNA, 16 bp, Artificial sequence
  Synthetic oligonucleotide
aacccggtaattttaa

SEQ ID NO: 310 - DNA, 16 bp, Artificial sequence
  Synthetic oligonucleotide
tacaagttttggacca

SEQ ID NO: 311 - DNA, 16 bp, Artificial sequence
  Synthetic oligonucleotide
ggctagattccctaag

SEQ ID NO: 312 - DNA, 16 bp, Artificial sequence
  Synthetic oligonucleotide
gtgaaaactaccgaag

SEQ ID NO: 313 - DNA, 16 bp, Artificial sequence
  Synthetic oligonucleotide
gacgcatacaaaacat

SEQ ID NO: 314 - DNA, 16 bp, Artificial sequence
  Synthetic oligonucleotide
tatactcaactgtgtc

SEQ ID NO: 315 - DNA, 16 bp, Artificial sequence
  Synthetic oligonucleotide
accttactaaaggacc

SEQ ID NO: 316 - DNA, 16 bp, Artificial sequence
  Synthetic oligonucleotide
gaccttactaaaggac

SEQ ID NO: 317 - DNA, 16 bp, Artificial sequence
  Synthetic oligonucleotide
gtttacccaataatac

SEQ ID NO: 318 - DNA, 16 bp, Artificial sequence
  Synthetic oligonucleotide
cactagtactgatgtc

SEQ ID NO: 319 - DNA, 16 bp, Artificial sequence
  Synthetic oligonucleotide
gtaagaacggtaataa

SEQ ID NO: 320 - DNA, 16 bp, Artificial sequence
  Synthetic oligonucleotide
atatggaagagcccta

SEQ ID NO: 321 - DNA, 16 bp, Artificial sequence
  Synthetic oligonucleotide
gcaatttaggtggtgc

SEQ ID NO: 322 - DNA, 16 bp, Artificial sequence
  Synthetic oligonucleotide
ctaatcaggagtatgc

SEQ ID NO: 323 - DNA, 16 bp, Artificial sequence
  Synthetic oligonucleotide
aaacccggtaatttta

SEQ ID NO: 324 - DNA, 16 bp, Artificial sequence
  Synthetic oligonucleotide
ctacaagttttggacc

SEQ ID NO: 325 - DNA, 16 bp, Artificial sequence
  Synthetic oligonucleotide
tgataatggaccccaa

SEQ ID NO: 326 - DNA, 16 bp, Artificial sequence
  Synthetic oligonucleotide
gtttttaaacgggttt

SEQ ID NO: 327 - DNA, 16 bp, Artificial sequence
  Synthetic oligonucleotide
aaactaccgaagttgt

SEQ ID NO: 328 - DNA, 16 bp, Artificial sequence
  Synthetic oligonucleotide
cactagccatccttac

SEQ ID NO: 329 - DNA, 16 bp, Artificial sequence
  Synthetic oligonucleotide
aaacattggccgcaaa

SEQ ID NO: 330 - DNA, 16 bp, Artificial sequence
  Synthetic oligonucleotide
ataaaggagttgcacc

SEQ ID NO: 331 - DNA, 16 bp, Artificial sequence
  Synthetic oligonucleotide
taacagatgcgcaaac

SEQ ID NO: 332 - DNA, 16 bp, Artificial sequence
  Synthetic oligonucleotide
caactgagggagcctt

SEQ ID NO: 333 - DNA, 16 bp, Artificial sequence
  Synthetic oligonucleotide
cgttaatagttaatag

SEQ ID NO: 334 - DNA, 16 bp, Artificial sequence
  Synthetic oligonucleotide
gataaaggagttgcac

SEQ ID NO: 335 - DNA, 16 bp, Artificial sequence
  Synthetic oligonucleotide
ataatgatgaatgtcg

SEQ ID NO: 336 - DNA, 16 bp, Artificial sequence
  Synthetic oligonucleotide
ataacagatgcgcaaa

SEQ ID NO: 337 - DNA, 16 bp, Artificial sequence
  Synthetic oligonucleotide
ggtaagagtcattttg

SEQ ID NO: 338 - DNA, 16 bp, Artificial sequence
  Synthetic oligonucleotide
taatcaggagtatgct

SEQ ID NO: 339 - DNA, 16 bp, Artificial sequence
  Synthetic oligonucleotide
cctaatcaggagtatg

SEQ ID NO: 340 - DNA, 16 bp, Artificial sequence
  Synthetic oligonucleotide
cttactaaaggacctc

SEQ ID NO: 341 - DNA, 16 bp, Artificial sequence
  Synthetic oligonucleotide
ccttactaaaggacct

SEQ ID NO: 342 - DNA, 16 bp, Artificial sequence
  Synthetic oligonucleotide
ggctagactttattat

SEQ ID NO: 343 - DNA, 16 bp, Artificial sequence
  Synthetic oligonucleotide
ggtaaggctagacttt

SEQ ID NO: 344 - DNA, 16 bp, Artificial sequence
  Synthetic oligonucleotide
cctaaatagaggtatg

SEQ ID NO: 345 - DNA, 16 bp, Artificial sequence
  Synthetic oligonucleotide
atgctttagatcaagc

SEQ ID NO: 346 - DNA, 16 bp, Artificial sequence
  Synthetic oligonucleotide
tgcaatattgttaacg

SEQ ID NO: 347 - DNA, 16 bp, Artificial sequence
  Synthetic oligonucleotide
ttaaaccaggtggaac

SEQ ID NO: 348 - DNA, 16 bp, Artificial sequence
  Synthetic oligonucleotide
gtacataatcaggatg

SEQ ID NO: 349 - DNA, 16 bp, Artificial sequence
  Synthetic oligonucleotide
gtggaaaggttatggc

SEQ ID NO: 350 - DNA, 16 bp, Artificial sequence
  Synthetic oligonucleotide
gctaatggtgattctg

SEQ ID NO: 351 - DNA, 16 bp, Artificial sequence
  Synthetic oligonucleotide
gaaaactaccgaagtt

SEQ ID NO: 352 - DNA, 16 bp, Artificial sequence
  Synthetic oligonucleotide
gacaatattgctttgc

SEQ ID NO: 353 - DNA, 16 bp, Artificial sequence
  Synthetic oligonucleotide
accaaattggctacta

SEQ ID NO: 354 - DNA, 16 bp, Artificial sequence
  Synthetic oligonucleotide
tcctaatcaggagtat

SEQ ID NO: 355 - DNA, 16 bp, Artificial sequence
  Synthetic oligonucleotide
ttactaaaggacctca

SEQ ID NO: 356 - DNA, 16 bp, Artificial sequence
  Synthetic oligonucleotide
aatgttggactgagac

SEQ ID NO: 357 - DNA, 16 bp, Artificial sequence
  Synthetic oligonucleotide
ccctactataactcaa

SEQ ID NO: 358 - DNA, 16 bp, Artificial sequence
  Synthetic oligonucleotide
cgcatacaaaacattc

SEQ ID NO: 359 - DNA, 16 bp, Artificial sequence
  Synthetic oligonucleotide
tatatggaagagccct

SEQ ID NO: 360 - DNA, 16 bp, Artificial sequence
  Synthetic oligonucleotide
cataacagatgcgcaa

SEQ ID NO: 361 - DNA, 16 bp, Artificial sequence
  Synthetic oligonucleotide
gccaaaaggcttctac

SEQ ID NO: 362 - DNA, 16 bp, Artificial sequence
  Synthetic oligonucleotide
atcctaatcaggagta

SEQ ID NO: 363 - DNA, 16 bp, Artificial sequence
  Synthetic oligonucleotide
tacccaataatactgc

SEQ ID NO: 364 - DNA, 16 bp, Artificial sequence
  Synthetic oligonucleotide
caaacccggtaatttt

SEQ ID NO: 365 - DNA, 16 bp, Artificial sequence
  Synthetic oligonucleotide
tgtaaacttacatagc

SEQ ID NO: 366 - DNA, 16 bp, Artificial sequence
  Synthetic oligonucleotide
tggtaccacatatatc

SEQ ID NO: 367 - DNA, 16 bp, Artificial sequence
  Synthetic oligonucleotide
tccctaagagtgatgg

SEQ ID NO: 368 - DNA, 16 bp, Artificial sequence
  Synthetic oligonucleotide
gtacaactattgttaa

SEQ ID NO: 369 - DNA, 16 bp, Artificial sequence
  Synthetic oligonucleotide
ggccaaactgtcacta

SEQ ID NO: 370 - DNA, 16 bp, Artificial sequence
  Synthetic oligonucleotide
caatttaggtggtgct

SEQ ID NO: 371 - DNA, 16 bp, Artificial sequence
  Synthetic oligonucleotide
gcaaaatgttggactg

SEQ ID NO: 372 - DNA, 16 bp, Artificial sequence
  Synthetic oligonucleotide
cctacaagttttggac

SEQ ID NO: 373 - DNA, 16 bp, Artificial sequence
  Synthetic oligonucleotide
ctaactaccaacatga

SEQ ID NO: 374 - DNA, 16 bp, Artificial sequence
  Synthetic oligonucleotide
cattagggaggacttg

SEQ ID NO: 375 - DNA, 16 bp, Artificial sequence
  Synthetic oligonucleotide
taacattagggaggac

SEQ ID NO: 376 - DNA, 16 bp, Artificial sequence
  Synthetic oligonucleotide
aacaatggaacctagt

SEQ ID NO: 377 - DNA, 16 bp, Artificial sequence
  Synthetic oligonucleotide
catacatcaccagatg

SEQ ID NO: 378 - DNA, 16 bp, Artificial sequence
  Synthetic oligonucleotide
ttacaaacattggccg

SEQ ID NO: 379 - DNA, 16 bp, Artificial sequence
  Synthetic oligonucleotide
taaaggagttgcacca

SEQ ID NO: 380 - DNA, 16 bp, Artificial sequence
  Synthetic oligonucleotide
tgcaatttaggtggtg

SEQ ID NO: 381 - DNA, 16 bp, Artificial sequence
  Synthetic oligonucleotide
gcataatgtctgatag

SEQ ID NO: 382 - DNA, 16 bp, Artificial sequence
  Synthetic oligonucleotide
tactggtaagagtcat

SEQ ID NO: 383 - DNA, 16 bp, Artificial sequence
  Synthetic oligonucleotide
gaggtatgagctatta

SEQ ID NO: 384 - DNA, 16 bp, Artificial sequence
  Synthetic oligonucleotide
tgttaaaccaggtgga

SEQ ID NO: 385 - DNA, 16 bp, Artificial sequence
  Synthetic oligonucleotide
cacctacaagttttgg

SEQ ID NO: 386 - DNA, 16 bp, Artificial sequence
  Synthetic oligonucleotide
gctaacttttgtgcac

SEQ ID NO: 387 - DNA, 16 bp, Artificial sequence
  Synthetic oligonucleotide
acctagtaataggttt

SEQ ID NO: 388 - DNA, 16 bp, Artificial sequence
  Synthetic oligonucleotide
atggaagagccctaat

SEQ ID NO: 389 - DNA, 16 bp, Artificial sequence
  Synthetic oligonucleotide
ctggtaaaggccaaca

SEQ ID NO: 390 - DNA, 16 bp, Artificial sequence
  Synthetic oligonucleotide
caggagtatgctgatg

SEQ ID NO: 391 - DNA, 16 bp, Artificial sequence
  Synthetic oligonucleotide
aatcaggagtatgctg

SEQ ID NO: 392 - DNA, 16 bp, Artificial sequence
  Synthetic oligonucleotide
catacaatgctagtta

SEQ ID NO: 393 - DNA, 16 bp, Artificial sequence
  Synthetic oligonucleotide
cactatatgttaaacc

SEQ ID NO: 394 - DNA, 16 bp, Artificial sequence
  Synthetic oligonucleotide
gcaaagaatagagctc

SEQ ID NO: 395 - DNA, 16 bp, Artificial sequence
  Synthetic oligonucleotide
tctaactaccaacatg

SEQ ID NO: 396 - DNA, 16 bp, Artificial sequence
  Synthetic oligonucleotide
ctaaatagaggtatgg

SEQ ID NO: 397 - DNA, 16 bp, Artificial sequence
  Synthetic oligonucleotide
acaacctaaatagagg

SEQ ID NO: 398 - DNA, 16 bp, Artificial sequence
  Synthetic oligonucleotide
gactttaaccttgtgg

SEQ ID NO: 399 - DNA, 16 bp, Artificial sequence
  Synthetic oligonucleotide
aacctagtaataggtt

SEQ ID NO: 400 - DNA, 16 bp, Artificial sequence
  Synthetic oligonucleotide
gaacctagtaataggt

SEQ ID NO: 401 - DNA, 16 bp, Artificial sequence
  Synthetic oligonucleotide
acagattgaaccagct

SEQ ID NO: 402 - DNA, 16 bp, Artificial sequence
  Synthetic oligonucleotide
atttaggtggtgctgt

SEQ ID NO: 403 - DNA, 16 bp, Artificial sequence
  Synthetic oligonucleotide
gcttatgctaatagtg

SEQ ID NO: 404 - DNA, 16 bp, Artificial sequence
  Synthetic oligonucleotide
ccccaaggtttaccca

SEQ ID NO: 405 - DNA, 16 bp, Artificial sequence
  Synthetic oligonucleotide
caacctaaatagaggt

SEQ ID NO: 406 - DNA, 16 bp, Artificial sequence
  Synthetic oligonucleotide
tgaaaactaccgaagt

SEQ ID NO: 407 - DNA, 16 bp, Artificial sequence
  Synthetic oligonucleotide
gtaataggtttcctat

SEQ ID NO: 408 - DNA, 16 bp, Artificial sequence
  Synthetic oligonucleotide
ttaaacaacttagctc

SEQ ID NO: 409 - DNA, 16 bp, Artificial sequence
  Synthetic oligonucleotide
tggtaaaggccaacaa

SEQ ID NO: 410 - DNA, 16 bp, Artificial sequence
  Synthetic oligonucleotide
gcatttgagctttggg

SEQ ID NO: 411 - DNA, 16 bp, Artificial sequence
  Synthetic oligonucleotide
gttaaaccaggtggaa

SEQ ID NO: 412 - DNA, 16 bp, Artificial sequence
  Synthetic oligonucleotide
taaacttacatagctc

SEQ ID NO: 413 - DNA, 16 bp, Artificial sequence
  Synthetic oligonucleotide
gtaaacttacatagct

SEQ ID NO: 414 - DNA, 16 bp, Artificial sequence
  Synthetic oligonucleotide
tacaatggttcaccat

SEQ ID NO: 415 - DNA, 16 bp, Artificial sequence
  Synthetic oligonucleotide
agtaataggtttccta

SEQ ID NO: 416 - DNA, 16 bp, Artificial sequence
  Synthetic oligonucleotide
tagtaataggtttcct

SEQ ID NO: 417 - DNA, 16 bp, Artificial sequence
  Synthetic oligonucleotide
gaacaatggaacctag

SEQ ID NO: 418 - DNA, 16 bp, Artificial sequence
  Synthetic oligonucleotide
aatcatacatcaccag

SEQ ID NO: 419 - DNA, 16 bp, Artificial sequence
  Synthetic oligonucleotide
cataatgtctgataga

SEQ ID NO: 420 - DNA, 16 bp, Artificial sequence
  Synthetic oligonucleotide
gactatggtgatgctg

SEQ ID NO: 421 - DNA, 16 bp, Artificial sequence
  Synthetic oligonucleotide
atatgttaaaccaggt

SEQ ID NO: 422 - DNA, 16 bp, Artificial sequence
  Synthetic oligonucleotide
tatatgttaaaccagg

SEQ ID NO: 423 - DNA, 16 bp, Artificial sequence
  Synthetic oligonucleotide
gtaacattagggagga

SEQ ID NO: 424 - DNA, 16 bp, Artificial sequence
  Synthetic oligonucleotide
acgcatacaaaacatt

SEQ ID NO: 425 - DNA, 16 bp, Artificial sequence
  Synthetic oligonucleotide
caaggaacaacattgc

SEQ ID NO: 426 - DNA, 16 bp, Artificial sequence
  Synthetic oligonucleotide
ctatatgttaaaccag

SEQ ID NO: 427 - DNA, 16 bp, Artificial sequence
  Synthetic oligonucleotide
ctctatctgtagtact

SEQ ID NO: 428 - DNA, 16 bp, Artificial sequence
  Synthetic oligonucleotide
gtgcaaagaatagagc

SEQ ID NO: 429 - DNA, 16 bp, Artificial sequence
  Synthetic oligonucleotide
atcttaagtatgccat

SEQ ID NO: 430 - DNA, 16 bp, Artificial sequence
  Synthetic oligonucleotide
cctactataactcaaa

SEQ ID NO: 431 - DNA, 16 bp, Artificial sequence
  Synthetic oligonucleotide
aacaatgtgtgatatc

SEQ ID NO: 432 - DNA, 16 bp, Artificial sequence
  Synthetic oligonucleotide
ctaatggtgattctga

SEQ ID NO: 433 - DNA, 16 bp, Artificial sequence
  Synthetic oligonucleotide
caactattgttaatgg

SEQ ID NO: 434 - DNA, 16 bp, Artificial sequence
  Synthetic oligonucleotide
ggacaatcaaaaagag

SEQ ID NO: 435 - DNA, 16 bp, Artificial sequence
  Synthetic oligonucleotide
gccctaatgtgtaaaa

SEQ ID NO: 436 - DNA, 16 bp, Artificial sequence
  Synthetic oligonucleotide
tactcaataatttggg

SEQ ID NO: 437 - DNA, 16 bp, Artificial sequence
  Synthetic oligonucleotide
cacaagagcactatgt

SEQ ID NO: 438 - DNA, 16 bp, Artificial sequence
  Synthetic oligonucleotide
ttaagtatgccattag

SEQ ID NO: 439 - DNA, 16 bp, Artificial sequence
  Synthetic oligonucleotide
cttaagtatgccatta

SEQ ID NO: 440 - DNA, 16 bp, Artificial sequence
  Synthetic oligonucleotide
tttttaaacgggtttg

SEQ ID NO: 441 - DNA, 16 bp, Artificial sequence
  Synthetic oligonucleotide
taaatagaggtatggt

SEQ ID NO: 442 - DNA, 16 bp, Artificial sequence
  Synthetic oligonucleotide
aacaacctaaatagag

SEQ ID NO: 443 - DNA, 16 bp, Artificial sequence
  Synthetic oligonucleotide
gtggaaaatcctacca

SEQ ID NO: 444 - DNA, 16 bp, Artificial sequence
  Synthetic oligonucleotide
taagatggagagcctt

SEQ ID NO: 445 - DNA, 16 bp, Artificial sequence
  Synthetic oligonucleotide
caccaacagagcctaa

SEQ ID NO: 446 - DNA, 16 bp, Artificial sequence
  Synthetic oligonucleotide
actcaataatttgggt

SEQ ID NO: 447 - DNA, 16 bp, Artificial sequence
  Synthetic oligonucleotide
ttctagcaatgttgca

SEQ ID NO: 448 - DNA, 16 bp, Artificial sequence
  Synthetic oligonucleotide
tttatgaggctatgta

SEQ ID NO: 449 - DNA, 16 bp, Artificial sequence
  Synthetic oligonucleotide
taaggctagactttat

SEQ ID NO: 450 - DNA, 16 bp, Artificial sequence
  Synthetic oligonucleotide
gttaaacaacttagct

SEQ ID NO: 451 - DNA, 16 bp, Artificial sequence
  Synthetic oligonucleotide
gaaaagtgtgaccttc

SEQ ID NO: 452 - DNA, 16 bp, Artificial sequence
  Synthetic oligonucleotide
tgctagggagagctgc

SEQ ID NO: 453 - DNA, 16 bp, Artificial sequence
  Synthetic oligonucleotide
ctacacaggcacctac

SEQ ID NO: 454 - DNA, 16 bp, Artificial sequence
  Synthetic oligonucleotide
cactagaaccagaata

SEQ ID NO: 455 - DNA, 16 bp, Artificial sequence
  Synthetic oligonucleotide
ccacaagagcactatg

SEQ ID NO: 456 - DNA, 16 bp, Artificial sequence
  Synthetic oligonucleotide
ctgctaacttttgtgc

SEQ ID NO: 457 - DNA, 16 bp, Artificial sequence
  Synthetic oligonucleotide
aggtaagatggagagc

SEQ ID NO: 458 - DNA, 16 bp, Artificial sequence
  Synthetic oligonucleotide
gtccagaacaaaccca

SEQ ID NO: 459 - DNA, 16 bp, Artificial sequence
  Synthetic oligonucleotide
aatcttaagtatgcca

SEQ ID NO: 460 - DNA, 16 bp, Artificial sequence
  Synthetic oligonucleotide
ccaacaatgtgtgata

SEQ ID NO: 461 - DNA, 16 bp, Artificial sequence
  Synthetic oligonucleotide
gacttgaaagagccac

SEQ ID NO: 462 - DNA, 16 bp, Artificial sequence
  Synthetic oligonucleotide
atggtaatgctttaga

SEQ ID NO: 463 - DNA, 16 bp, Artificial sequence
  Synthetic oligonucleotide
ggaaaatcctaccata

SEQ ID NO: 464 - DNA, 16 bp, Artificial sequence
  Synthetic oligonucleotide
caacgagaaaacacac

SEQ ID NO: 465 - DNA, 16 bp, Artificial sequence
  Synthetic oligonucleotide
cattactttatgatgc

SEQ ID NO: 466 - DNA, 16 bp, Artificial sequence
  Synthetic oligonucleotide
acaacaaggccaaact

SEQ ID NO: 467 - DNA, 16 bp, Artificial sequence
  Synthetic oligonucleotide
aatttaggtggtgctg

SEQ ID NO: 468 - DNA, 16 bp, Artificial sequence
  Synthetic oligonucleotide
gatcaagatgcacttt

SEQ ID NO: 469 - DNA, 16 bp, Artificial sequence
  Synthetic oligonucleotide
acattatcaacaatgc

SEQ ID NO: 470 - DNA, 16 bp, Artificial sequence
  Synthetic oligonucleotide
tctctatgagaaccaa

SEQ ID NO: 471 - DNA, 18 bp, Artificial sequence
  Synthetic oligonucleotide
gtttgttacctgggaagg

SEQ ID NO: 472 - DNA, 18 bp, Artificial sequence
  Synthetic oligonucleotide
ggtttgttacctgggaag

SEQ ID NO: 473 - DNA, 18 bp, Artificial sequence
  Synthetic oligonucleotide
tggtttgttacctgggaa

SEQ ID NO: 474 - DNA, 18 bp, Artificial sequence
  Synthetic oligonucleotide
ttggtttgttacctggga

SEQ ID NO: 475 - DNA, 18 bp, Artificial sequence
  Synthetic oligonucleotide
gttggtttgttacctggg

SEQ ID NO: 476 - DNA, 18 bp, Artificial sequence
  Synthetic oligonucleotide
ggttggtttgttacctgg

SEQ ID NO: 477 - DNA, 18 bp, Artificial sequence
  Synthetic oligonucleotide
tggttggtttgttacctg

SEQ ID NO: 478 - DNA, 18 bp, Artificial sequence
  Synthetic oligonucleotide
ttggttggtttgttacct

SEQ ID NO: 479 - DNA, 18 bp, Artificial sequence
  Synthetic oligonucleotide
gttggttggtttgttacc

SEQ ID NO: 480 - DNA, 18 bp, Artificial sequence
  Synthetic oligonucleotide
agttggttggtttgttac

SEQ ID NO: 481 - DNA, 18 bp, Artificial sequence
  Synthetic oligonucleotide
aagttggttggtttgtta

SEQ ID NO: 482 - DNA, 18 bp, Artificial sequence
  Synthetic oligonucleotide
aaagttggttggtttgtt

SEQ ID NO: 483 - DNA, 18 bp, Artificial sequence
  Synthetic oligonucleotide
gaaagttggttggtttgt

SEQ ID NO: 484 - DNA, 18 bp, Artificial sequence
  Synthetic oligonucleotide
cgaaagttggttggtttg

SEQ ID NO: 485 - DNA, 18 bp, Artificial sequence
  Synthetic oligonucleotide
tcgaaagttggttggttt

SEQ ID NO: 486 - DNA, 18 bp, Artificial sequence
  Synthetic oligonucleotide
atcgaaagttggttggtt

SEQ ID NO: 487 - DNA, 18 bp, Artificial sequence
  Synthetic oligonucleotide
gatcgaaagttggttggt

SEQ ID NO: 488 - DNA, 18 bp, Artificial sequence
  Synthetic oligonucleotide
agatcgaaagttggttgg

SEQ ID NO: 489 - DNA, 18 bp, Artificial sequence
  Synthetic oligonucleotide
gagatcgaaagttggttg

SEQ ID NO: 490 - DNA, 18 bp, Artificial sequence
  Synthetic oligonucleotide
agagatcgaaagttggtt

SEQ ID NO: 491 - DNA, 18 bp, Artificial sequence
  Synthetic oligonucleotide
aagagatcgaaagttggt

SEQ ID NO: 492 - DNA, 18 bp, Artificial sequence
  Synthetic oligonucleotide
caagagatcgaaagttgg

SEQ ID NO: 493 - DNA, 18 bp, Artificial sequence
  Synthetic oligonucleotide
acaagagatcgaaagttg

SEQ ID NO: 494 - DNA, 18 bp, Artificial sequence
  Synthetic oligonucleotide
tacaagagatcgaaagtt

SEQ ID NO: 495 - DNA, 18 bp, Artificial sequence
  Synthetic oligonucleotide
ctacaagagatcgaaagt

SEQ ID NO: 496 - DNA, 18 bp, Artificial sequence
  Synthetic oligonucleotide
tctacaagagatcgaaag

SEQ ID NO: 497 - DNA, 18 bp, Artificial sequence
  Synthetic oligonucleotide
atctacaagagatcgaaa

SEQ ID NO: 498 - DNA, 18 bp, Artificial sequence
  Synthetic oligonucleotide
gatctacaagagatcgaa

SEQ ID NO: 499 - DNA, 18 bp, Artificial sequence
  Synthetic oligonucleotide
agatctacaagagatcga

SEQ ID NO: 500 - DNA, 18 bp, Artificial sequence
  Synthetic oligonucleotide
cagatctacaagagatcg

SEQ ID NO: 501 - DNA, 18 bp, Artificial sequence
  Synthetic oligonucleotide
acagatctacaagagatc

SEQ ID NO: 502 - DNA, 18 bp, Artificial sequence
  Synthetic oligonucleotide
aacagatctacaagagat

SEQ ID NO: 503 - DNA, 18 bp, Artificial sequence
  Synthetic oligonucleotide
gaacagatctacaagaga

SEQ ID NO: 504 - DNA, 18 bp, Artificial sequence
  Synthetic oligonucleotide
agaacagatctacaagag

SEQ ID NO: 505 - DNA, 18 bp, Artificial sequence
  Synthetic oligonucleotide
gagaacagatctacaaga

SEQ ID NO: 506 - DNA, 18 bp, Artificial sequence
  Synthetic oligonucleotide
agagaacagatctacaag

SEQ ID NO: 507 - DNA, 18 bp, Artificial sequence
  Synthetic oligonucleotide
tagagaacagatctacaa

SEQ ID NO: 508 - DNA, 18 bp, Artificial sequence
  Synthetic oligonucleotide
ttagagaacagatctaca

SEQ ID NO: 509 - DNA, 18 bp, Artificial sequence
  Synthetic oligonucleotide
tttagagaacagatctac

SEQ ID NO: 510 - DNA, 18 bp, Artificial sequence
  Synthetic oligonucleotide
gtttagagaacagatcta

SEQ ID NO: 511 - DNA, 20 bp, Artificial sequence
  Synthetic oligonucleotide
tggtttgttacctgggaagg

SEQ ID NO: 512 - DNA, 20 bp, Artificial sequence
  Synthetic oligonucleotide
ttggtttgttacctgggaag

SEQ ID NO: 513 - DNA, 20 bp, Artificial sequence
  Synthetic oligonucleotide
gttggtttgttacctgggaa

SEQ ID NO: 514 - DNA, 20 bp, Artificial sequence
  Synthetic oligonucleotide
ggttggtttgttacctggga

SEQ ID NO: 515 - DNA, 20 bp, Artificial sequence
  Synthetic oligonucleotide
tggttggtttgttacctggg

SEQ ID NO: 516 - DNA, 20 bp, Artificial sequence
  Synthetic oligonucleotide
ttggttggtttgttacctgg

SEQ ID NO: 517 - DNA, 20 bp, Artificial sequence
  Synthetic oligonucleotide
gttggttggtttgttacctg

SEQ ID NO: 518 - DNA, 20 bp, Artificial sequence
  Synthetic oligonucleotide
agttggttggtttgttacct

SEQ ID NO: 519 - DNA, 20 bp, Artificial sequence
  Synthetic oligonucleotide
aagttggttggtttgttacc

SEQ ID NO: 520 - DNA, 20 bp, Artificial sequence
  Synthetic oligonucleotide
aaagttggttggtttgttac

SEQ ID NO: 521 - DNA, 20 bp, Artificial sequence
  Synthetic oligonucleotide
gaaagttggttggtttgtta

SEQ ID NO: 522 - DNA, 20 bp, Artificial sequence
  Synthetic oligonucleotide
cgaaagttggttggtttgtt

SEQ ID NO: 523 - DNA, 20 bp, Artificial sequence
  Synthetic oligonucleotide
tcgaaagttggttggtttgt

SEQ ID NO: 524 - DNA, 20 bp, Artificial sequence
  Synthetic oligonucleotide
atcgaaagttggttggtttg

SEQ ID NO: 525 - DNA, 20 bp, Artificial sequence
  Synthetic oligonucleotide
gatcgaaagttggttggttt

SEQ ID NO: 526 - DNA, 20 bp, Artificial sequence
  Synthetic oligonucleotide
agatcgaaagttggttggtt

SEQ ID NO: 527 - DNA, 20 bp, Artificial sequence
  Synthetic oligonucleotide
gagatcgaaagttggttggt

SEQ ID NO: 528 - DNA, 20 bp, Artificial sequence
  Synthetic oligonucleotide
agagatcgaaagttggttgg

SEQ ID NO: 529 - DNA, 20 bp, Artificial sequence
  Synthetic oligonucleotide
aagagatcgaaagttggttg

SEQ ID NO: 530 - DNA, 20 bp, Artificial sequence
  Synthetic oligonucleotide
caagagatcgaaagttggtt

SEQ ID NO: 531 - DNA, 20 bp, Artificial sequence
  Synthetic oligonucleotide
acaagagatcgaaagttggt

SEQ ID NO: 532 - DNA, 20 bp, Artificial sequence
  Synthetic oligonucleotide
tacaagagatcgaaagttgg

SEQ ID NO: 533 - DNA, 20 bp, Artificial sequence
  Synthetic oligonucleotide
ctacaagagatcgaaagttg

SEQ ID NO: 534 - DNA, 20 bp, Artificial sequence
  Synthetic oligonucleotide
tctacaagagatcgaaagtt

SEQ ID NO: 535 - DNA, 20 bp, Artificial sequence
  Synthetic oligonucleotide
atctacaagagatcgaaagt

SEQ ID NO: 536 - DNA, 20 bp, Artificial sequence
  Synthetic oligonucleotide
gatctacaagagatcgaaag

SEQ ID NO: 537 - DNA, 20 bp, Artificial sequence
  Synthetic oligonucleotide
agatctacaagagatcgaaa

SEQ ID NO: 538 - DNA, 20 bp, Artificial sequence
  Synthetic oligonucleotide
cagatctacaagagatcgaa

SEQ ID NO: 539 - DNA, 20 bp, Artificial sequence
  Synthetic oligonucleotide
acagatctacaagagatcga

SEQ ID NO: 540 - DNA, 20 bp, Artificial sequence
  Synthetic oligonucleotide
aacagatctacaagagatcg

SEQ ID NO: 541 - DNA, 20 bp, Artificial sequence
  Synthetic oligonucleotide
gaacagatctacaagagatc

SEQ ID NO: 542 - DNA, 20 bp, Artificial sequence
  Synthetic oligonucleotide
agaacagatctacaagagat

SEQ ID NO: 543 - DNA, 20 bp, Artificial sequence
  Synthetic oligonucleotide
gagaacagatctacaagaga

SEQ ID NO: 544 - DNA, 20 bp, Artificial sequence
  Synthetic oligonucleotide
agagaacagatctacaagag

SEQ ID NO: 545 - DNA, 20 bp, Artificial sequence
  Synthetic oligonucleotide
tagagaacagatctacaaga

SEQ ID NO: 546 - DNA, 20 bp, Artificial sequence
  Synthetic oligonucleotide
ttagagaacagatctacaag

SEQ ID NO: 547 - DNA, 20 bp, Artificial sequence
  Synthetic oligonucleotide
tttagagaacagatctacaa

SEQ ID NO: 548 - DNA, 20 bp, Artificial sequence
  Synthetic oligonucleotide
gtttagagaacagatctaca

SEQ ID NO: 549 - DNA, 16 bp, Artificial sequence
  Synthetic oligonucleotide
ttgttacctgggaagg

SEQ ID NO: 550 - DNA, 16 bp, Artificial sequence
  Synthetic oligonucleotide
tttgttacctgggaag

SEQ ID NO: 551 - DNA, 16 bp, Artificial sequence
  Synthetic oligonucleotide
gtttgttacctgggaa

SEQ ID NO: 552 - DNA, 16 bp, Artificial sequence
  Synthetic oligonucleotide
ggtttgttacctggga

SEQ ID NO: 553 - DNA, 16 bp, Artificial sequence
  Synthetic oligonucleotide
tggtttgttacctggg

SEQ ID NO: 554 - DNA, 16 bp, Artificial sequence
  Synthetic oligonucleotide
ttggtttgttacctgg

SEQ ID NO: 555 - DNA, 16 bp, Artificial sequence
  Synthetic oligonucleotide
gttggtttgttacctg

SEQ ID NO: 556 - DNA, 16 bp, Artificial sequence
  Synthetic oligonucleotide
ggttggtttgttacct

SEQ ID NO: 557 - DNA, 16 bp, Artificial sequence
  Synthetic oligonucleotide
tggttggtttgttacc

SEQ ID NO: 558 - DNA, 16 bp, Artificial sequence
  Synthetic oligonucleotide
ttggttggtttgttac

SEQ ID NO: 559 - DNA, 16 bp, Artificial sequence
  Synthetic oligonucleotide
gttggttggtttgtta

SEQ ID NO: 560 - DNA, 16 bp, Artificial sequence
  Synthetic oligonucleotide
agttggttggtttgtt

SEQ ID NO: 561 - DNA, 16 bp, Artificial sequence
  Synthetic oligonucleotide
aagttggttggtttgt

SEQ ID NO: 562 - DNA, 16 bp, Artificial sequence
  Synthetic oligonucleotide
aaagttggttggtttg

SEQ ID NO: 563 - DNA, 16 bp, Artificial sequence
  Synthetic oligonucleotide
gaaagttggttggttt

SEQ ID NO: 564 - DNA, 16 bp, Artificial sequence
  Synthetic oligonucleotide
cgaaagttggttggtt

SEQ ID NO: 565 - DNA, 16 bp, Artificial sequence
  Synthetic oligonucleotide
tcgaaagttggttggt

SEQ ID NO: 566 - DNA, 16 bp, Artificial sequence
  Synthetic oligonucleotide
atcgaaagttggttgg

SEQ ID NO: 567 - DNA, 16 bp, Artificial sequence
  Synthetic oligonucleotide
gatcgaaagttggttg

SEQ ID NO: 568 - DNA, 16 bp, Artificial sequence
  Synthetic oligonucleotide
agatcgaaagttggtt

SEQ ID NO: 569 - DNA, 16 bp, Artificial sequence
  Synthetic oligonucleotide
gagatcgaaagttggt

SEQ ID NO: 570 - DNA, 16 bp, Artificial sequence
  Synthetic oligonucleotide
agagatcgaaagttgg

SEQ ID NO: 571 - DNA, 16 bp, Artificial sequence
  Synthetic oligonucleotide
aagagatcgaaagttg

SEQ ID NO: 572 - DNA, 16 bp, Artificial sequence
  Synthetic oligonucleotide
caagagatcgaaagtt

SEQ ID NO: 573 - DNA, 16 bp, Artificial sequence
  Synthetic oligonucleotide
acaagagatcgaaagt

SEQ ID NO: 574 - DNA, 16 bp, Artificial sequence
  Synthetic oligonucleotide
tacaagagatcgaaag

SEQ ID NO: 575 - DNA, 16 bp, Artificial sequence
  Synthetic oligonucleotide
ctacaagagatcgaaa

SEQ ID NO: 576 - DNA, 16 bp, Artificial sequence
  Synthetic oligonucleotide
tctacaagagatcgaa

SEQ ID NO: 577 - DNA, 16 bp, Artificial sequence
  Synthetic oligonucleotide
atctacaagagatcga

SEQ ID NO: 578 - DNA, 16 bp, Artificial sequence
  Synthetic oligonucleotide
gatctacaagagatcg

SEQ ID NO: 579 - DNA, 16 bp, Artificial sequence
  Synthetic oligonucleotide
agatctacaagagatc

SEQ ID NO: 580 - DNA, 16 bp, Artificial sequence
  Synthetic oligonucleotide
cagatctacaagagat

SEQ ID NO: 581 - DNA, 16 bp, Artificial sequence
  Synthetic oligonucleotide
acagatctacaagaga

SEQ ID NO: 582 - DNA, 16 bp, Artificial sequence
  Synthetic oligonucleotide
aacagatctacaagag

SEQ ID NO: 583 - DNA, 16 bp, Artificial sequence
  Synthetic oligonucleotide
gaacagatctacaaga

SEQ ID NO: 584 - DNA, 16 bp, Artificial sequence
  Synthetic oligonucleotide
agaacagatctacaag

SEQ ID NO: 585 - DNA, 16 bp, Artificial sequence
  Synthetic oligonucleotide
gagaacagatctacaa

SEQ ID NO: 586 - DNA, 16 bp, Artificial sequence
  Synthetic oligonucleotide
agagaacagatctaca

SEQ ID NO: 587 - DNA, 16 bp, Artificial sequence
  Synthetic oligonucleotide
tagagaacagatctac

SEQ ID NO: 588 - DNA, 16 bp, Artificial sequence
  Synthetic oligonucleotide
ttagagaacagatcta

SEQ ID NO: 589 - DNA, 20 bp, Artificial sequence
  Synthetic oligonucleotide
aaagttcgtttagagaacag

SEQ ID NO: 590 - DNA, 20 bp, Artificial sequence
  Synthetic oligonucleotide
taaagttcgtttagagaaca

SEQ ID NO: 591 - DNA, 20 bp, Artificial sequence
  Synthetic oligonucleotide
ttaaagttcgtttagagaac

SEQ ID NO: 592 - DNA, 20 bp, Artificial sequence
  Synthetic oligonucleotide
tttaaagttcgtttagagaa

SEQ ID NO: 593 - DNA, 20 bp, Artificial sequence
  Synthetic oligonucleotide
ttttaaagttcgtttagaga

SEQ ID NO: 594 - DNA, 20 bp, Artificial sequence
  Synthetic oligonucleotide
attttaaagttcgtttagag

SEQ ID NO: 595 - DNA, 20 bp, Artificial sequence
  Synthetic oligonucleotide
gattttaaagttcgtttaga

SEQ ID NO: 596 - DNA, 20 bp, Artificial sequence
  Synthetic oligonucleotide
agattttaaagttcgtttag

SEQ ID NO: 597 - DNA, 20 bp, Artificial sequence
  Synthetic oligonucleotide
aagttcgtttagagaacaga

SEQ ID NO: 598 - DNA, 20 bp, Artificial sequence
  Synthetic oligonucleotide
gttcgtttagagaacagatc

SEQ ID NO: 599 - DNA, 20 bp, Artificial sequence
  Synthetic oligonucleotide
agttcgtttagagaacagat

SEQ ID NO: 600 - DNA, 16 bp, Artificial sequence
  Synthetic oligonucleotide
cgccgataaggtacac